(19) United States
(12) Patent Application Publication
(10) Pub. No.: US 2024/0279588 A1
(43) Pub. Date: Aug. 22, 2024

(54) SYSTEMS, DEVICES, AND METHODS FOR PARALLEL WORKFLOWS IN AUTOMATED CELL PROCESSING

(71) Applicant: Cellares Corporation, South San Francisco, CA (US)

(72) Inventors: Daniele MALLEO, Menlo Park, CA (US); Marc ELPEL, Poulsbo, WA (US)

(21) Appl. No.: 18/444,426

(22) Filed: Feb. 16, 2024

Related U.S. Application Data
(60) Provisional application No. 63/446,718, filed on Feb. 17, 2023.

(51) Int. Cl. C12M 1/36; C12M 1/00; C12M 1/12; C12M 1/26; C12M 1/42; C12M 3/00

(52) U.S. Cl. CPC C12M 41/48; C12M 23/40; C12M 23/42; C12M 23/44; C12M 29/06; C12M 33/04; C12M 35/02; C12M 37/02

(57) ABSTRACT

Disclosed herein are cell processing systems, devices, and methods thereof. A system for planning parallel biomanufacturing processes comprises a plurality of cartridges, each containing a cell sample, a biomanufacturing workcell configured to receive the plurality of cartridges and comprising a plurality of instruments configured to interface with respective modules of each one of the plurality of cartridges to perform biomanufacturing processes on the cell sample therein, and a computing device comprising a processor configured to receive one or more inputs associated with the biomanufacturing processes to be performed on the cell sample within each of the plurality of cartridges, simulate the biomanufacturing processes based on the received one or more inputs, identify potential contentions based on the simulation, and generate an output based on the one or more inputs associated with the biomanufacturing processes.

### CROSS-REFERENCE TO RELATED APPLICATIONS

This application claims the benefit of U.S. Provisional Patent Application No. 63/446,718, filed Feb. 17, 2023, the content of which is herein incorporated by reference in its entirety.

### TECHNICAL FIELD

Devices, systems, and methods herein relate to high-throughput manufacturing of cell products for biomedical applications using automated systems executing parallel workflows.

### BACKGROUND

Cellular therapies based on hematopoietic stem cells, chimeric antigen receptor T cells, NK cells, tumor infiltrating lymphocytes, T-cell receptors, regulatory T cells, gamma delta T cells, and others rely on manufacturing of cell products. Manufacturing of such cell products typically involves multiple cell processing steps. Conventional solutions for manufacturing cell products typically rely on single cartridge processes. That is, conventional approaches are configured to generate a defined amount of one cellular therapy at any given time. As a result, these approaches are unable to accommodate tasks requiring concurrent production of more than one type of cellular therapy and/or large quantities of a same type of cellular therapy. Because these single cartridge processes are not capable of processing multiple cartridges at once, cell processing for critical therapies is particularly time consuming, and usually low throughput.

Applicant has previously invented a workcell with multiple instruments, where the workcell is capable of handling multiple cartridges in parallel (see e.g., U.S. Pat. No. 11,376,587). Applicant has found that the complexity of parallel processing, which is made possible by the workcell, can result in contentions and conflicts. Accordingly, approaches for addressing, among other things, the contention issues that are unique to the concurrent processing of multiple cartridges, while optimizing workflows and improving throughput are desirable.

### SUMMARY

The present disclosure relates generally to methods and systems for processing cell products. By processing a cell product in a cartridge moved between instruments, some embodiments may achieve one or more advantages over prior cell manufacturing systems, including, for example, improved sterility, automation, lower cost of goods, lower labor costs, higher repeatability, higher reliability, lower risk of operator error, lower risk of contamination, higher process flexibility, higher capacity, higher instrument throughput, higher degree of process scalability, and shorter process duration. Embodiments of the disclosure may comprise a sterile enclosure, thereby reducing the costs of providing a clean room environment, and/or utilize a workcell having a smaller footprint than current manufacturing facilities. Furthermore, embodiments of the methods disclosed herein may, in some cases, be performed more quickly and with less risk of cell product loss.

In an embodiment, the present disclosure further relates to a system for planning parallel biomanufacturing processes, the system comprising a plurality of cartridges, each containing a cell sample, a biomanufacturing workcell configured to receive the plurality of cartridges and comprising a plurality of instruments configured to interface with respective modules of each one of the plurality of cartridges to perform biomanufacturing processes on the cell sample therein, and a computing device comprising a processor configured to receive one or more inputs associated with the biomanufacturing processes to be performed on the cell sample within each of the plurality of cartridges, simulate the biomanufacturing processes based on the received one or more inputs, identify potential contentions based on the simulation, and generate an output based on the one or more inputs associated with the biomanufacturing processes, the simulation, and the identified potential contentions, wherein the output comprises a chart including a displayed series of intervals, wherein each interval represents a displayed series of time periods, and wherein the plurality of instruments are displayed as a function of the displayed series of interval and the displayed series of time periods.

In an embodiment, the present disclosure further relates to a method for identifying workflow conflicts in parallel biomanufacturing processes, the system comprising receiving, by a processor of a computing device, one or more inputs associated with biomanufacturing processes to be performed on cell samples within each of a plurality of cartridges, simulating the parallel biomanufacturing processes based on the received one or more inputs, identifying potential contentions based on the simulation, and generating an output based on the simulated parallel biomanufacturing process and the identified potential contentions, wherein the output comprises a chart wherein a plurality of instruments are displayed as a function of a series of intervals and a series of time periods, wherein the parallel biomanufacturing processes are performed on the cell samples within the plurality of cartridges in coordination with a biomanufacturing workcell comprising a plurality of instruments configured to interface with respective modules of each one of the plurality of cartridges to perform the biomanufacturing processes.

Additional embodiments, features, and advantages of the invention will be apparent from the following detailed description and through practice of the invention.

### BRIEF DESCRIPTION OF THE DRAWINGS

FIG. 1A is a block diagram of an illustrative embodiment of a cell processing system.

FIG. 1B is a block diagram of an illustrative embodiment of a cartridge of a cell processing system.

FIG. 2A is a block diagram of an illustrative embodiment of a cell processing system. FIG. 2B is a perspective view of an illustrative embodiment of a workcell of a cell processing system. FIG. 2C is a perspective view of an illustrative embodiment of a workcell and cartridge of a cell processing system.

FIG. 2D is a block diagram of an illustrative embodiment of a cell processing system. FIG. 2E is a block diagram of an illustrative embodiment of a cell processing system.

FIG. 3 is a block diagram of an illustrative embodiment of a cell processing system.

FIG. 4A is a perspective view of an illustrative embodiment of a cell processing system. FIG. 4B is an additional perspective view of an illustrative embodiment of a cell processing system.

FIG. 5 is a perspective view of an illustrative embodiment of a cell processing system.

FIG. 6 is a flowchart of an illustrative embodiment of a method of cell processing.

FIG. 7 is a flowchart of an illustrative embodiment of a method of cell processing.

FIG. 8 is a flowchart of an illustrative embodiment of a method of cell processing.

FIG. 9 is a flowchart of an illustrative embodiment of a method of executing a transformation model.

FIG. 10 depicts a high-level arrangement of an exemplary model illustrating the interactions between a user, a cartridge, and a workcell comprising instruments such as a bioreactor, a feedthrough, a transcription factors system, a cell separation system, a sterile liquid transfer system, and a reagent vault system.

FIG. 11A is a flow diagram of an embodiment of a method for planning parallel workflows in a cell processing system. FIG. 11B is a flow diagram of an embodiment of a method for planning parallel workflows in a cell processing system. FIG. 11C is a flow diagram of a sub process of a method for planning parallel workflows in a cell processing system. FIG. 11D is an image depicting a step of a sub process of a method for planning parallel workflows in a cell processing system. FIG. 11E is an image depicting a step of a sub process of a method for planning parallel workflows in a cell processing system. FIG. 11F is an image depicting a step of a sub process of a method for planning parallel workflows in a cell processing system. FIG. 11G is a flow diagram of an embodiment of a method for planning parallel workflows in a cell processing system.

FIG. 12 is a tabular depiction of inputs to the method for planning parallel workflows in a cell processing system.

FIG. 13 is an exemplary periodic Gantt chart generated for a single cartridge according to methods of the present disclosure.

FIG. 14 is an exemplary periodic Gantt chart generated for three cartridges according to methods of the present disclosure.

FIG. 15A is a periodic Gantt chart generated for a single cartridge demonstrating a workflow that likely generates contentions upon the introduction of additional cartridges, as resources (i.e., instruments become constrained). FIG. 15B is an exemplary periodic Gantt chart generated for a single cartridge where instrument usage is planned so that additional cartridges can be introduced without contending for resource usage.

FIG. 16 is an exemplary periodic Gantt chart generated for 14 cartridges according to methods of the present disclosure, demonstrating zero contentions as a result of planning.

FIG. 17A is a graphical depiction of a position of two cartridges, sequentially introduced, relative to instruments of a workcell.

FIG. 17B is a graphical depiction of a position of two cartridges (C1, C2) relative to instruments of a workcell, wherein workflows of the two cartridges overlap.

FIG. 18 is an exemplary graphical user interface which may display periodic Gantt charts generated according to methods of the present disclosure.

FIG. 19 is an illustrative embodiment of a graphical user interface relating to an initial process design interface.

FIG. 20 is an illustrative embodiment of a graphical user interface relating to creating a process.

FIG. 21 is an illustrative embodiment of a graphical user interface relating to an empty process.

FIG. 22 is an illustrative embodiment of a graphical user interface relating to adding a reagent and a container.

FIG. 23 is an illustrative embodiment of a graphical user interface relating to a process parameter.

FIG. 24 is an illustrative embodiment of a graphical user interface relating to a patient weight process parameter.

FIG. 25 is an illustrative embodiment of a graphical user interface relating to a preprocess analytic.

FIG. 26 is an illustrative embodiment of a graphical user interface relating to a white blood cell count preprocess analytic.

FIG. 27 is an illustrative embodiment of a graphical user interface relating to process parameter calculation.

FIG. 28 is an illustrative embodiment of a graphical user interface relating to a completed process setup.

FIG. 29 is an illustrative embodiment of a graphical user interface relating to process operations activation settings.

FIG. 30 is an illustrative embodiment of a graphical user interface relating to a filled process operations activation settings.

FIG. 31 is an illustrative embodiment of a graphical user interface relating to an initial process operations.

FIG. 32 is an illustrative embodiment of a graphical user interface relating to dragging in process operations.

FIG. 33 is an illustrative embodiment of a graphical user interface relating to dragging in process operations.

FIG. 34 is an illustrative embodiment of a graphical user interface relating to filled process operations.

FIG. 35 is an illustrative embodiment of a graphical user interface relating to product monitoring.

FIG. 36 is an illustrative embodiment of a graphical user interface relating to product monitoring.

FIG. 37 is a block diagram of an illustrative embodiment of a manufacturing workflow.

### DETAILED DESCRIPTION

### Definitions

As used herein, the singular forms “a”, “an” and “the” include plural referents unless the context clearly dictates otherwise. “And” as used herein is interchangeably used with “or” unless expressly stated otherwise. Words using the singular or plural number also include the plural and singular number, respectively. Additionally, the words “herein,” “above,” “below,” and words of similar import, when used in this application, shall refer to this application as a whole and not to any particular portions of the application.

As used herein, a “cell processing system” can comprise a “consumable” and a “workcell”, among other features. The “workcell” may also be referred to herein, and/or in the accompany Drawings, as a “cell shuttle”. Generally, the term “consumable” can be used to represent a number of components that can be introduced to the “workcell”, such as a “cartridge” and/or another fluid device (e.g., a sterile liquid transfer device including one or more types of reagents, buffers, cytokines, or other fluids) that may be introduced to the workcell. In embodiments, each “consumable” can be sterilized and reused for the production of additional cell therapy products. The systems and methods of the present disclosure, though applicable to any consumable or any combination of consumables, will be described herein, for clarity, in the context of a cartridge.

Throughout this application, the term “about” is used to indicate that a value includes the inherent variation of error for the device or the method being employed to determine the value, or the variation that exists among the samples being measured. Unless otherwise stated or otherwise evident from the context, the term “about” means within 10% above or below the reported numerical value (except where such number would exceed 100% of a possible value or go below 0%). When used in conjunction with a range or series of values, the term “about” applies to the endpoints of the range or each of the values enumerated in the series, unless otherwise indicated. As used in this application, the terms “about” and “approximately” are used as equivalents.

### Cell Processing System

A particular challenge of a shared instrumentation system, an example of which is the cell processing system of the present disclosure, is realized when multiple cartridge workflows, featuring multiple different processes (each with different inherent variabilities), are orchestrated over time with the requirement that none of the cartridge workflows fails. In other words, in a shared instrumentation system, it is challenging to ensure that each workflow is successfully executed as a function of system ability to schedule shared resources. This combination of process variability and required/known outcomes are fundamentally at odds when working to ensure that all cartridges can move through e.g., a biomanufacturing process without contention and/or deadlock.

Efforts to handle this problem have met some of these challenges, but not all. These include standard timing analysis, which often include non-periodic Gantt plotting, as well as other approaches to modeling process variation, including simple minimum-typical-maximum timing (3-possible state modeling) and broad input Monte Carlo simulations have been attempted. However, these approaches often fail in permitting practical analyses on parallel workflows.

3-state modeling, for instance, though appropriate for capturing worst case scenarios, is unable to model parallel workflows within e.g. a workcell in a practicable manner. Standard timing analysis, though able to provide certain insights on cartridge loading, is unable to solve for the problem of parallel workflows, particularly when the number of concurrent cartridges and the variability of processes performed thereon increases. Moreover, with regard to identifying scheduling conflicts, Gantt viewing provides limited insight, as inherent to the process of generating a Gantt chart is methods of slipping timing of by-locked processes to eliminate contention timing. As a result, events viewed in a Gantt chart, which plots processes by time, will appear as though they occur in sequence, illustrating times of heavy usage but not the amount of contention that occurred during the simulation.

When considering multiple cartridges in parallel workflows, the task of modeling every possible simulation permutation becomes increasingly challenging and impracticable. For instance, assuming 50 cartridges are to be evaluated, the equation to verify that the outcome of all cartridges will be successful for a given input workflow and cartridge introduction rate is 125,000,000,000 simulations. With further consideration to other cartridge introduction rates (e.g., sweeping the introduction rate over a range) renders another multiplier that must be added to the simulations. As such, while previous models may be able to provide useful insights into loading rates and associated contention issues, they lack the ability to address the fundamental requirement that no cartridge shall fail as a function of the system's ability to schedule shared resources when the number of cartridges being processed in parallel increases.

These concerns can be further exemplified in the context of the cell processing system of the present disclosure. The workcell of the cell processing system is comprised of multiple instruments (e.g., bioreactors, centrifuges, elutriators, cell separators, sterile liquid transfer instruments—either independently or combined into a multi-functional instrument)—for performing a cell manufacturing process. Cartridges containing cell samples for cell processing may be introduced to the workcell. In some embodiments, the cartridges can be transported and placed (e.g., via one or more robotic arms) in different locations within the workcell (e.g., feed through, a liquid transfer docking station, a bioreactor docking station) as part of one or more steps in a cell manufacturing process.

As there can be multiple cartridges being processed in parallel by the various instruments, wherein each instrument has its own processing time (e.g., cell culturing and expansion in a bioreactor may take longer than cell separation), it can be a challenge, as highlighted above, to maximize or optimize the throughput of the system without any cartridge failing to successfully finish their workflow. For example, delays and deviations from a planned cell process can occur when there is a constraint, conflict, and/or deadlock that prevents a cartridge from being processed in a planned station (e.g., a docking station of an instrument may be occupied by a first cartridge, thereby delaying processing of a second cartridge within the docking station of the same instrument).

In view of the above, and in order to ensure 100% success for each cartridge and increase throughput, it can be appreciated that planning parallel workflows within the workcell of the present disclosure requires a new solution. Accordingly, as provided for herein, there is a need to provide systems and tools that assist in mitigating conflicts between cartridges, thereby increasing throughput of cartridges processed in parallel and optimizing planning.

The present disclosure describes a system, device, and method for planning parallel biomanufacturing processes. The present disclosure describes a simulator tool that enables modeling and analysis of cell processing system performance under various scenarios. To this end, the present disclosure utilizes block simulation within a discrete-event simulator which enables modeling and analysis of system performance under various scenarios including cartridge introduction rate and process workflow. Outputs of the discrete-event simulator (or the model) include, among others, throughput, instrument contention resulting from parallel processing, and cartridge utilization and timing.

In an embodiment, the discrete-event simulation of parallel workflows described herein relies on “Periodic Gantt Planning and Plotting”. Periodic Gantt Planning and Plotting features a “periodic” Gantt chart of a predetermined periodicity. As will be described herein, Periodic Gantt Planning and Plotting is a method of optimizing process inputs (e.g., workflows), which may be variable, and scheduling to create a deterministic process output.

In some embodiments, the cell processing system comprises a plurality of cartridges, each containing a cell sample, and a biomanufacturing workcell configured to receive the plurality of cartridges. The workcell may comprise a plurality of instruments configured to interface with respective modules of each one of the plurality of cartridges to perform biomanufacturing processes on the cell sample therein. The cell processing methods and systems described herein may comprise moving a cartridge containing a cell product between a plurality of instruments inside the workcell.

In some embodiments, a plurality of cell processing steps may be performed within a single cartridge. For example, a robotic arm may be configured to move a cartridge between instruments for different cell processing steps. The cartridge may comprise a plurality of modules (i.e., cell processing devices) such as a bioreactor, a counterflow centrifugal elutriation (CCE) module, a magnetic cell sorter (e.g., magnetic-activated cell selection module), an electroporation device (e.g., electroporation module), a sorting module (e.g., fluorescence activated cell sorting (FACS) module), an acoustic flow cell module, a centrifugation module, a microfluidic enrichment module, combinations thereof, and the like. In embodiments, the system processes two or more cartridges in parallel. In this way, the cell processing systems of the present disclosure may reduce operator intervention and increase throughput by automating cartridge (and cell product) movement between instruments and/or various cell processing steps using a robot.

In embodiments, the system further comprises a computing device comprising a processor configured to receive one or more inputs associated with biomanufacturing processes to be performed on the cell sample within each of the plurality of cartridges, simulate the biomanufacturing processes based on the received one or more inputs, identify potential contentions based on the simulation, and generate an output based on the one or more inputs associated with the biomanufacturing processes, the simulation, and the identified potential contentions. The output may comprise a chart including a displayed series of intervals, wherein each interval represents a displayed series of time periods, and wherein the plurality of instruments is displayed as a function of the displayed series of interval and the displayed series of time periods.

In some embodiments, the simulation may be performed by a continuous or analog simulator. In some embodiments, the simulation may be performed by a discrete-event simulator that models the operation of the cell processing system as a discrete sequence of events in time. With a discrete-event simulator, each event occurs at a particular instant in time and marks a change of state in the system. The discrete-event approach models a process as a series of instantaneous occurrences, or discrete events. In between these events, the system is approximated as fixed and unchanging. In some embodiments, the simulation may include additional sophistication such as modeling of inputs and/or process variability via Monte Carlo simulation. The application of discrete-event simulation to cell manufacturing systems advantageously enables the observation of system behavior and the optimization of resource allocation in a system with given constraints and targets.

### I. Cell Processing System—Structure

Described here are systems and apparatuses configured to perform cell processing steps to manufacture a cell product (e.g., cell therapy product). In some embodiments, a cell processing system may comprise a plurality of instruments each independently configured to perform one or more cell processing operations upon a cartridge, and a robot capable of moving the cartridge between each of the plurality of instruments. The use of a robot and controller may facilitate one or more of automation, efficiency, and sterility of a cell processing system. In some embodiments, the plurality of instruments may be within a workcell comprising an enclosure.

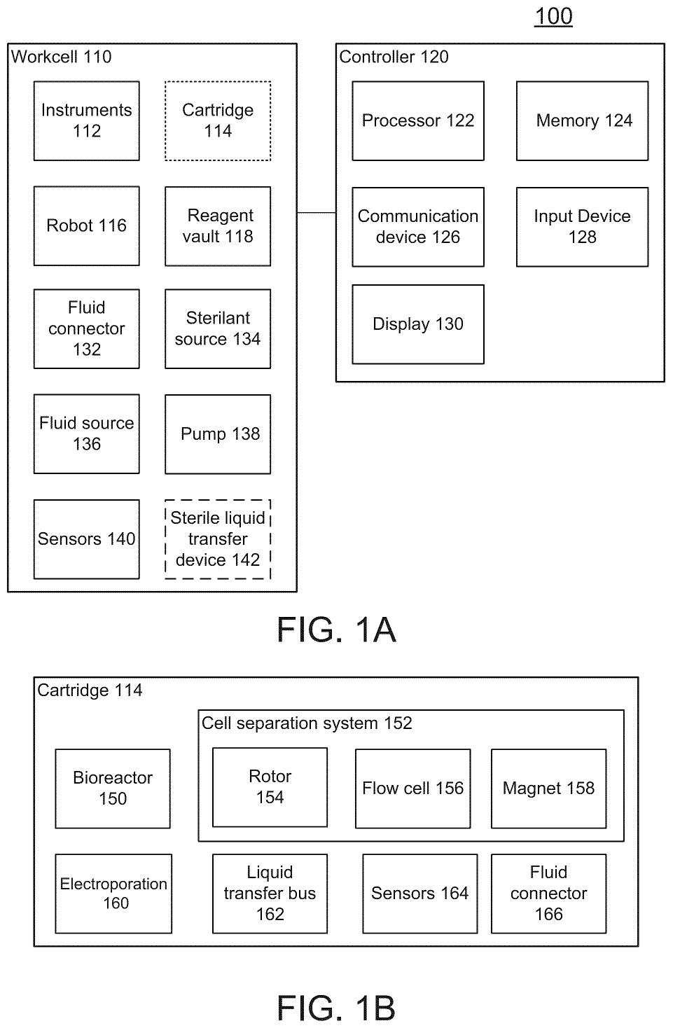

FIG. 1A is a block diagram of a cell processing system 100 comprising a workcell 110 and controller 120. In some embodiments, the workcell 110 may comprise one or more of an instrument 112, a robot 116 (e.g., robotic arm), a reagent vault 118, a fluid connector 132, a sterilant source 129, a fluid source 136, a pump 138, and a sensor 140. In embodiments, a cartridge 114 and a sterile liquid transfer device 142, part of the cell processing system 100, may be within the workcell 110. In some embodiments, the controller 120 may comprise one or more of a processor 122, a memory 124, a communication device 126, an input device 128, and a display 130.

In some embodiments, a workcell 110 may comprise a fully, or at least partially, enclosed housing inside which one or more cell processing steps are performed in a fully, or at least partially, automated process. In some embodiments, the workcell may be an open system lacking an enclosure, which may be configured for use in a clean room, a biosafety cabinet, or other sterile location. In some embodiments, the cartridge 114 may be moved using the robot 116 to reduce manual labor in the cell processing steps. In some embodiments, the workcell may be configured to perform sterile liquid transfers into and out of the cartridge in a fully or partially automated process. For example, one or more fluids may be stored in a sterile liquid transfer device 142. In some embodiments, the sterile liquid transfer device may be portable and may be moved within the system 100. The sterile liquid transfer devices and fluid connectors described herein enable the transfer of fluids in an automated, sterile, and metered manner for automating cell therapy manufacturing. In some embodiments, the enclosure of the workcell may be configured to meet International Organization for Standardization (ISO) standard ISO7 or better (e.g., ISO6 or ISO5). An advantage of meeting ISO7 or better standards is that the system may be used in a facility that does not meet ISO7 standards (i.e. that lack a clean room or other sufficiently filtered air space). Optionally, the facility may be an ISO8 or ISO9 facility. In some embodiments, a workcell may comprise a volume of less than about 800 m³, less than about 700 m³, less than about 600 m³, less than about 500 m³, less than about 300 m³, less than about 250 m³, less than about 200 m³, less than about 150 m³, less than about 100 m³, less than about 50 m³, less than about 25 m³, less than about 10 m³, and less than about 5 m³, including all ranges and sub-values in-between.

In some embodiments, a robot 116 may be configured to manipulate cartridges 114 and fluid connectors 132 between different instruments to perform a predetermined sequence of cell processing steps. In some embodiments, the same cartridge 114 may be received by different instruments 112 and/or, as is the case in the present disclosure, multiple cartridges 114 may be processed in parallel.

In some embodiments, a cartridge 114 may contain cell product intended for different recipients (e.g., in an allogenic workflow). The cell product from a single donor may be split between multiple cartridges 114 if necessary to generate enough product for therapeutic use, or when a donor is providing product for several recipients (e.g., for allogeneic transplant). The cell product for a single recipient may be split between multiple cartridges 114 if necessary to generate several cell products with unique genetic modifications, and then optionally recombined in certain ratios for therapeutic use in that recipient. For example, a fluid connector 132 may be coupled between two or more cartridges 114 to transfer a cell product and/or fluid between the cartridges 114. Furthermore, a fluid connector 132 may be coupled between any set of fluid-carrying components of the system 100 (e.g., cartridge 114, reagent vault 118, fluid source 136, sterile liquid transfer device 142, fluid conduit, container, vessel, etc.). For example, a first fluid connector may be coupled between a first cartridge and a sterile liquid transfer device, and a second fluid connector may be coupled between the sterile liquid transfer device and a second cartridge.

As illustrated in FIG. 1B, a cartridge 114 may comprise one or more of a bioreactor 150, a cell separation system 152, an electroporation module 160, a liquid transfer bus 162, a sensor 164, and a fluid connector 166, as described in more detail herein. A cell separation system 152 may comprise one or more of a rotor 154, flow cell 156, and magnet 158. In some embodiments, the magnet 158 may comprise one or more magnets and/or magnet arrays. For example, the cell separation system 152 may comprise a first magnet configured to magnetically rotate a rotor 154 and a second magnet (e.g., magnet array) configured to magnetically separate cells in flow cell 156.

### A. Workcell

In some embodiments, a workcell of a cell processing system, as introduced above with reference to FIG. 1A, may comprise a plurality of instruments each independently configured to perform one or more cell processing operations upon a cartridge of the cell processing system. A robot of the workcell may be configured to move the cartridge between each of the plurality of instruments. The instruments may comprise one or more of a bioreactor instrument, a cell selection instrument (e.g., a magnetic-activated cell selection instrument), a sorting instrument (e.g., a fluorescence activated cell sorting (FACS) instrument), an electroporation instrument, a counterflow centrifugal elutriation (CCE) instrument, a reagent vault, and the like. The workcell may perform automated manufacturing of cell products.

A cartridge may be configured to be portable and facilitate automated and sterile cell processing within a workcell. For example, the cartridge may be configured to move relative to one or more instruments of the workcell to perform different cell processing steps. In some embodiments, an instrument may be configured to move relative to a cartridge. In some embodiments, the cartridge may comprise a plurality of modules including one or more of a bioreactor module, a cell selection module (e.g., magnetic-activated cell selection module), a sorting module (e.g., fluorescence activated cell sorting (FACS) module), an electroporation module, and a counterflow centrifugal elutriation (CCE) module. The cartridge may further comprise one or more of a sterile liquid transfer port, a liquid transfer bus fluidically coupled to each module, and a pump fluidically coupled to the liquid transfer bus.

In some embodiments, a method of processing a solution containing a cell product may include the cell processing steps of digesting tissue using an enzyme reagent to release a select cell population into solution, enriching cells using a CCE instrument, washing cells using the CCE instrument, selecting cells in the solution using a selection instrument, sorting cells in the solution using a sorting instrument, differentiating or expanding the cells in a bioreactor, activating cells using an activating reagent, electroporating cells, transducing cells using a vector, and finishing a cell product.

In some embodiments, a workcell 110 may comprise at least a partially enclosed enclosure (e.g., housing) in which one or more automated cell processing steps are performed. For example, the workcell 110 may be configured to transfer sterile liquid into and out of a cartridge 114 in a fully or partially automated process. In some embodiments, a workcell 110 may not have an enclosure and be configured for use in a clean room, a biosafety cabinet, or other suitably clean or sterile location. In some embodiments, the workcell 100 may comprise a feedthrough access biosafety cabinet, quality control instrumentation, pump, fluid device, fluid connector, feedthrough, and sterilization system (e.g., sterilant source and/or generator, fluid source, heater/dessicator, aerator).

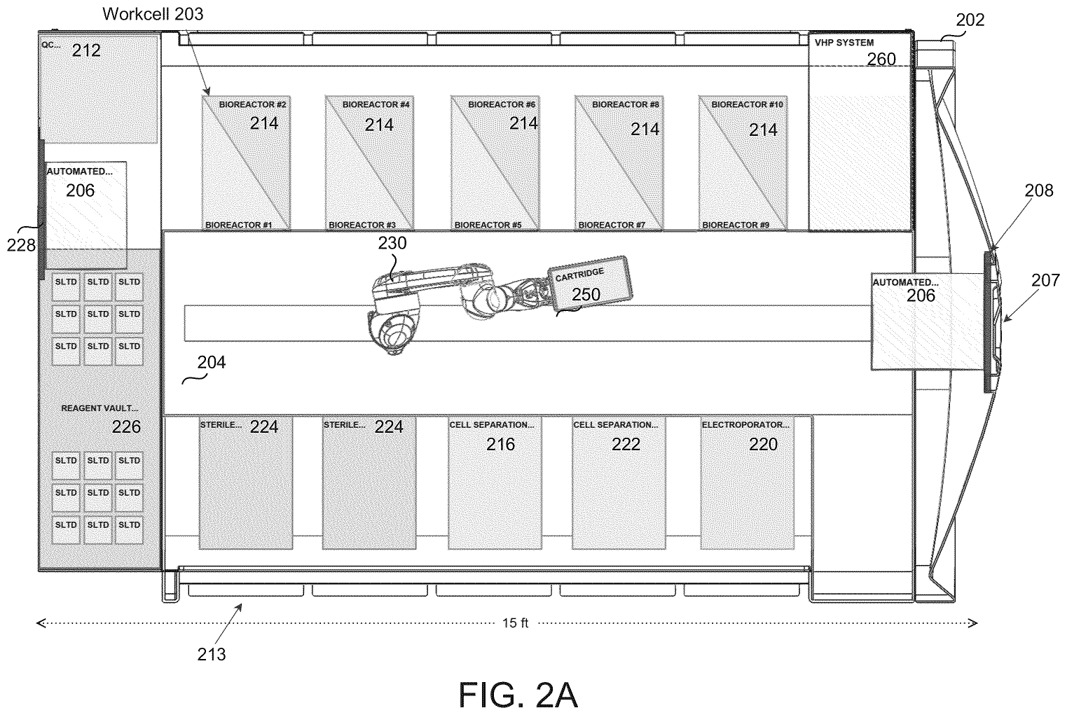

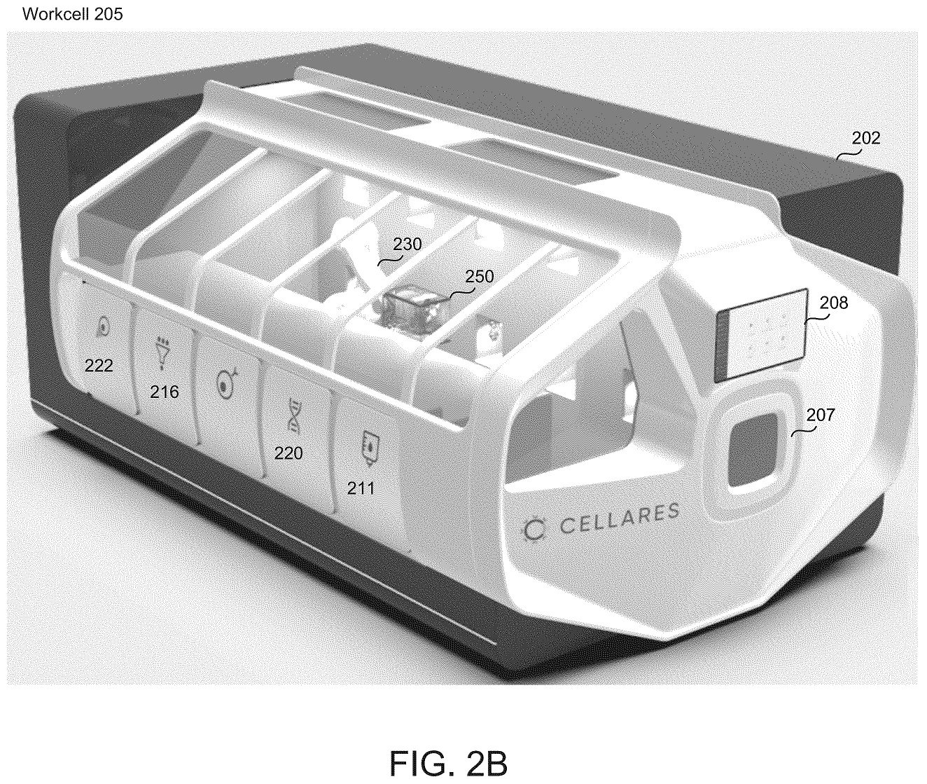

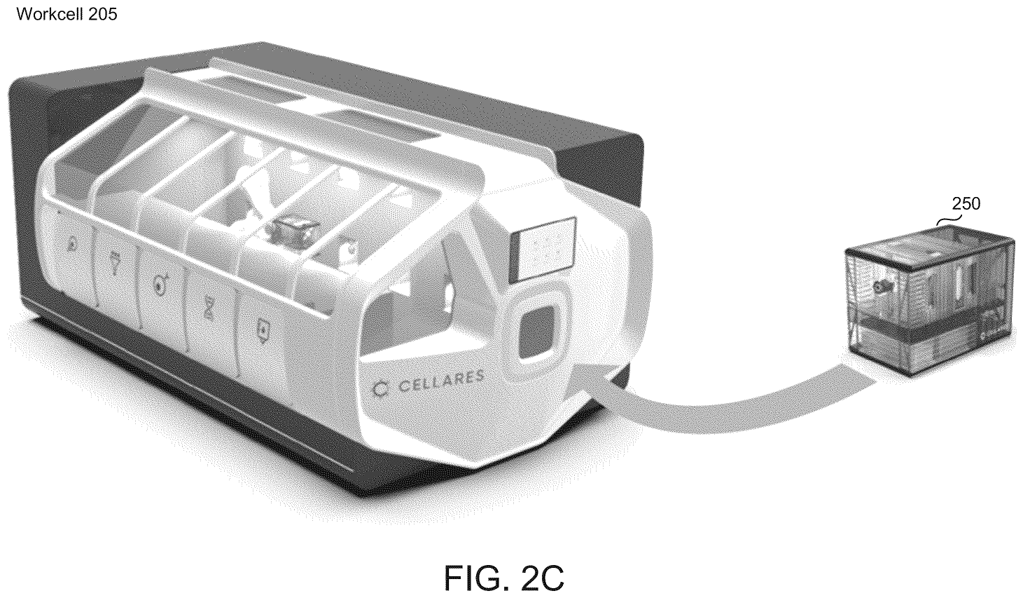

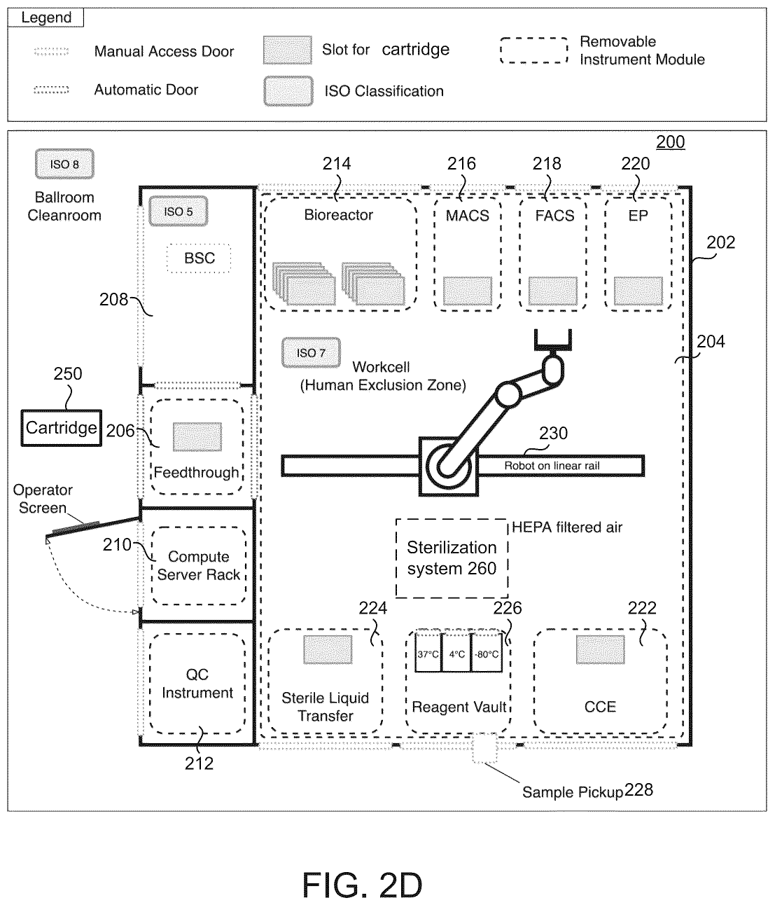

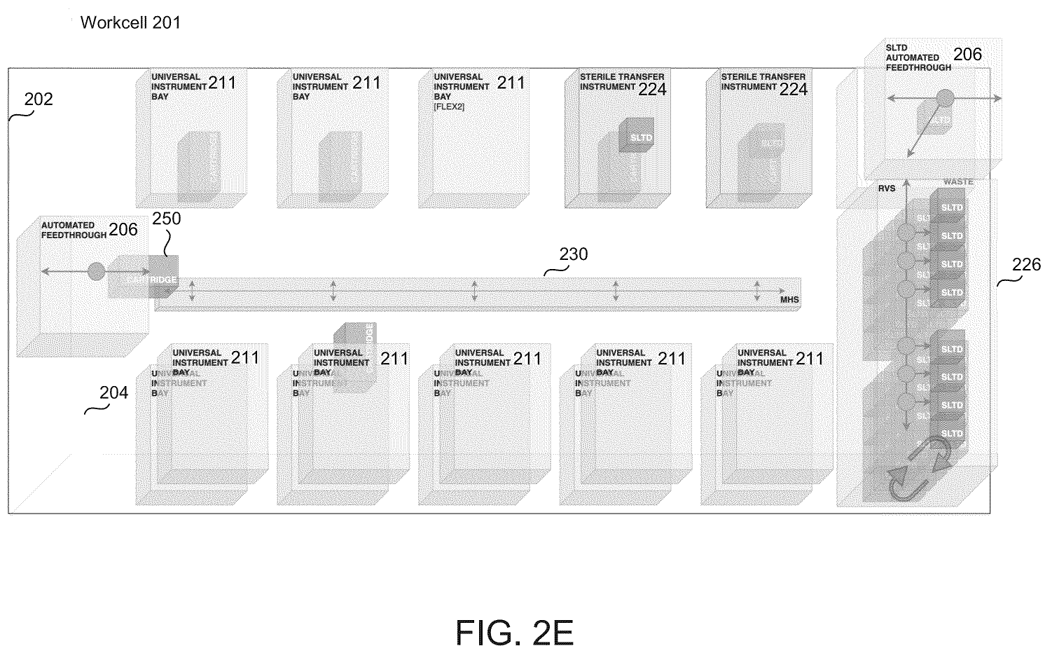

FIG. 2A is a block diagram of a cell processing system including a workcell 203. Workcell 203 may comprise an enclosure 202 having four walls, a base, and a roof. The workcell may be divided into an interior zone 204 with a feedthrough 206 access, and quality control (QC) instrumentation 212. An air filtration inlet (not shown) may provide high-efficiency particulate air (HEPA) filtration to provide ISO7 or better air quality in the interior zone 204. This air filtration may maintain sterile cell processing in an ISO8 or ISO9 manufacturing environment. The workcell 203 may also have an air filter on the air outlet to preserve the ISO rating of the room. In some embodiments, the workcell 203 may further comprise, inside the interior zone 104, a bioreactor instrument 214, a cell selection instrument 216 (e.g., MACS), an electroporation instrument (EP) 220, a counterflow centrifugation elutriation (CCE) instrument 222, a sterile liquid transfer instrument 224 (e.g., fluid connector), a reagent vault 226, and a sterilization system 260. The reagent vault 226 may be accessible by a user through a sample pickup port 228. A robot 230 (e.g., support arm, robotic arm) may be configured to move one or more cartridges 250 any instrument to any other instrument and/or move one or more cartridges 250 to and from a reagent vault. In some embodiments, the workcell 203 may comprise one or more moveable barrier 213 (e.g., access, door) configured to facilitate access to one or more of the instruments in the workcell 203.

In some embodiments of methods according to the disclosure, a human operator may load one or more empty cartridges 250 into the feedthrough 206 via cartridge port 207. The cartridges 250 may be pre-sterilized, or the feedthrough 206 may sterilize the cartridge 250 using ultraviolet radiation (UV), or chemical sterilizing agents provided as a vapor, spray, or wash. The feedthrough 206 chamber may optionally be configured to automatically spray, wash, irradiate, or otherwise treat cartridges (e.g. with ethanol and/or isopropyl alcohol solutions, vaporized hydrogen peroxide (VHP)) to maintain sterility of the interior zone 204 (e.g., ISO 7 or better). The cartridge 250 may be passed to the biosafety cabinet 206, where input cell product is provided and loaded to the cartridge through a sterile liquid transfer port into the cartridge 250. The user (via robot 230) may then move the cartridge 250 back to the feedthrough 206 and initiate automated processing using a computer processor in the computer server rack (e.g., controller 120). The robot 230 may be configured to move the cartridge 250 in a predefined sequence to a plurality of instruments and stations, with the components of the workcell 200. At the end of cell processing, the cartridge 250, now containing the processed cell product, may be returned to the feedthrough 206 for retrieval by the user. In some embodiments, an outer surface of the enclosure 202 may comprise an input/output device 208 (e.g., display, touchscreen).

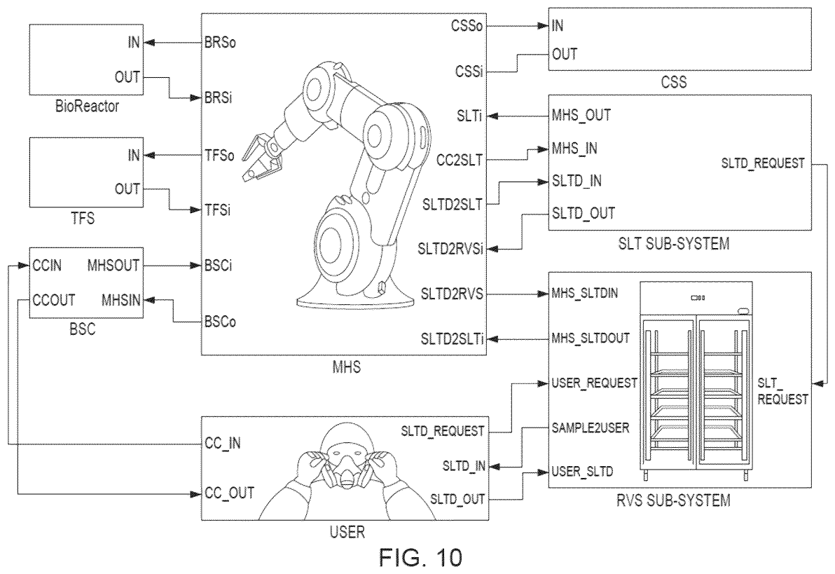

FIG. 2B is a perspective view of a workcell 205 of a cell processing system. FIG. 2C is a perspective view of a cell processing system depicting a cartridge 250 (e.g., any of the cartridges described herein) introduced into a workcell 205 (e.g., any of the workcells described herein). A plurality of cartridges may be inserted into the workcell 205 simultaneously and undergo one or more cell processing operations in parallel, as will be described later with reference to FIGS. 10-18.

In some embodiments, the workcell 205 may comprise a height of more than about a meter, between about 1 m and about 3 m, between about 1 m and about 5 m, between about 3, and about 10 m, between about 5 m and about 20 m, between about 10 m and about 30 m, between about 20 m and 100 m, and more than about 100 m, including all values and ranges in-between. In some embodiments, the workcell 205 may comprise one or more of a length and width of more than about 1 meter, between about 1 m and about 5 m, between about 3, and about 10 m, between about 5 m and about 20 m, between about 10 m and about 30 m, between about 20 m and 100 m, and more than about 100 m, including all values and ranges in-between.

FIG. 2D is a schematic illustration of an embodiment of a workcell 200. Workcell 200 may comprise an enclosure 202 having four walls, a base, and a roof. The workcell may be divided into an interior zone 204 with a feedthrough 206 access, a biosafety cabinet (BSC) 208, compute server rack 210 (e.g., controller 120), and quality control (QC) instrumentation 212. An air filtration inlet (not shown) may provide high-efficiency particulate air (HEPA) filtration to provide ISO7 or better air quality in the interior zone 204. This air filtration may maintain sterile cell processing in an ISO8 or ISO9 manufacturing environment. The workcell may also have an air filter on the air outlet to preserve the ISO rating of the room. In some embodiments, the workcell 200 may further comprise, inside the interior zone 204, an instrument 211 (e.g., disposed in a universal instrument bay), a bioreactor instrument 214, a cell selection instrument 216 (e.g., MACS, cell selection system), a cell sorting instrument 218 (e.g., FACS), an electroporation instrument (EP) 220, and a counterflow centrifugation elutriation (CCE) instrument 222, a sterile liquid transfer instrument 224 (e.g., fluid connector), a reagent vault 226, and a sterilization system 260 comprising one or more of a sterilant source, fluid source, and a pump. The reagent vault 226 may be accessible by a user through a sample pickup port 228. A robot 230 (e.g., support arm, robotic arm) may be configured to move one or more cartridges 250 from any instrument to any other instrument or reagent vault.

In some embodiments, a human operator may load one or more cartridges 250 into the feedthrough 206. The cartridges 250 may be pre-sterilized, or the feedthrough 206 may sterilize the cartridge 250 using ultraviolet radiation (UV), or chemical sterilizing agents provided as a spray or wash. The feedthrough 206 chamber may optionally be configured to automatically spray, wash, irradiate, or otherwise treat cartridges (e.g., with ethanol and/or isopropyl alcohol solutions) to maintain sterility of the interior zone 204 (e.g., ISO 7 or better) or the biosafety cabinet 208 (e.g., ISO 5 or better). The cartridge 250 may be passed to the biosafety cabinet 206, where input cell product is provided and loaded to the cartridge using a sterile liquid transfer instrument 224 (e.g., fluid connector) into the cartridge 250. The user may then move the cartridge 250 back to the feedthrough 206 and initiate automated processing using a computer processor in the computer server rack 210 (e.g., controller 120). The robot 230 may be configured to move the cartridge 250 in a predefined sequence to a plurality of instruments and stations, with the components of the workcell 200 being controlled by the computer processor of the computer server rack 210. Additionally, or alternatively, the sequence that the cartridge 250 moves within the workcell 200 may not be predefined. For example, cartridge 250 movement may not be dependent on one or more of the results of a previous step, sensor value, predetermined threshold (e.g., based on a quality control system), and the like. At the end of cell processing, the cartridge 250, now containing the processed cell product, may be returned to the feedthrough 206 for retrieval by the user. Additionally, or alternatively, the cartridge 250 containing the processed cell product may be transferred (via a fluid connector) to a second cartridge (e.g., single-use cartridge) and stored in the reagent vault 226 for retrieval by the user.

In some embodiments, cells from a patient and starting reagents may be loaded into a cartridge (e.g., single-use cartridge) by a human operator in a biosafety cabinet located separate from the workcell or integrated into the workcell. In some embodiments, the cartridges described herein comprising a cell product and reagent may move through a non-sterile field without contamination since the cartridge is closed. The cartridge may further undergo an automated decontamination routine. For example, the cartridge may be placed within a feedthrough capable of facilitating decontamination of the cartridge before entering the ISO 7 environment in the workcell.

FIG. 2E is a plan schematic illustration of another embodiment of a workcell 201. Workcell 201 may comprise an enclosure 202 having four walls, a base, and a roof. The workcell may be divided into an interior zone 204 with a feedthrough 206 access, a biosafety cabinet (BSC) 208, compute server rack 210 (e.g., controller 120), and quality control (QC) instrumentation 212. An air filtration inlet (not shown) may provide high-efficiency particulate air (HEPA) filtration to provide ISO7 or better air quality in the interior zone 204. This air filtration may maintain sterile cell processing in an ISO8 or ISO9 manufacturing environment. The workcell may also have an air filter on the air outlet to preserve the ISO rating of the room. In some embodiments, the workcell 200 may further comprise, inside the interior zone 104, an instrument 211 (e.g., disposed in a universal instrument bay), a bioreactor instrument 214, a cell selection instrument 216 (e.g., MACS), a cell sorting instrument 218 (e.g., FACS), an electroporation instrument (EP) 220, and a counterflow centrifugation elutriation (CCE) instrument 222, a sterile liquid transfer instrument 224, and a reagent vault 226. The reagent vault 226 may be accessible by a user through a sample pickup port 228 (e.g., a door which may facilitate bulk loading of sterile liquid transfer instruments 224). A robot 230 (e.g., support arm, robotic arm) may be configured to move one or more cartridges 250 from any instrument to any other instrument or reagent vault.

In some embodiments of methods according to the disclosure, a human operator may load one or more empty cartridges 250 into the feedthrough 206. Additionally, or alternatively, pre-filled cartridges may be loaded into the feedthrough 206. The cartridges 250 may be pre-sterilized, or the feedthrough 206 may sterilize the cartridge 250 using ultraviolet radiation (UV), or chemical sterilizing agents provided as a spray or wash. The feedthrough 206 chamber may optionally be configured to automatically spray, wash, irradiate, or otherwise treat cartridges (e.g., with ethanol and/or isopropyl alcohol solutions) to maintain sterility of the interior zone 204 (e.g., ISO 7 or better) or the biosafety cabinet 208 (e.g., ISO 5 or better). The cartridge 250 may be passed to the biosafety cabinet 106, where input cell product is provided and loaded to the cartridge through a sterile liquid transfer port into the cartridge 250. The user may then move the cartridge 250 back to the feedthrough 206 and initiate automated processing using a computer processor in the computer server rack 210 (e.g., controller 120). The robot 230 may be configured to move the cartridge 250 in a predefined sequence to a plurality of instruments and stations, with the components of the workcell 200 being controlled by the computer processor of the computer server rack 210. At the end of cell processing, the cartridge 250, now containing the processed cell product, may be returned to the feedthrough 206 for retrieval by the user.

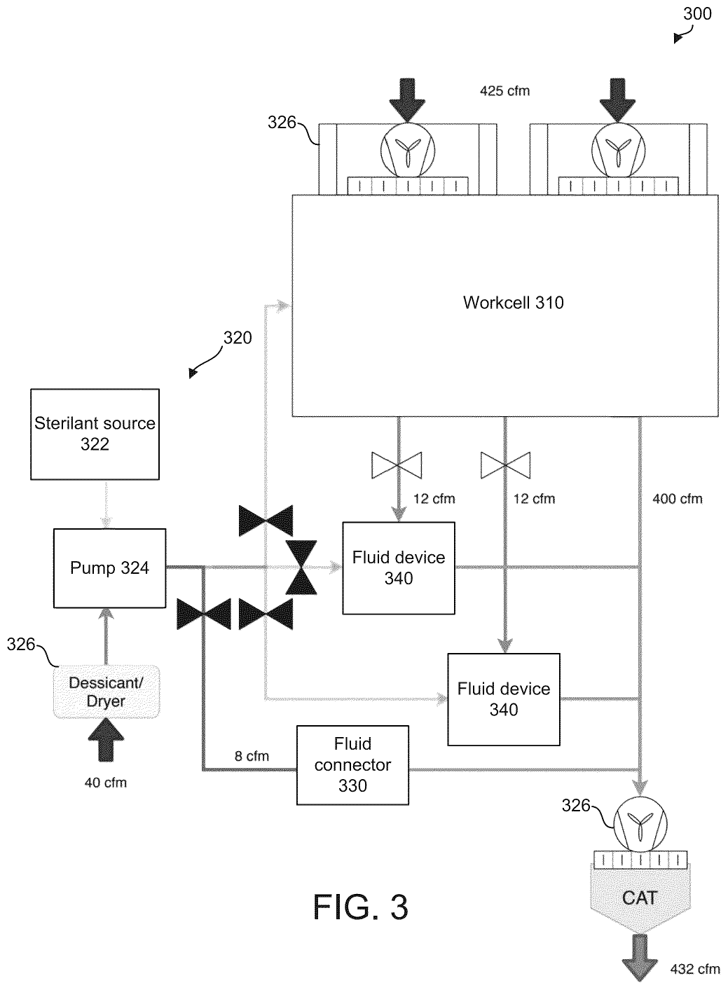

In some embodiments, one or more components of a sterilization system (e.g., sterilant source, pump) may be coupled to a workcell. For example, FIG. 3 is a block diagram of a cell processing system 300 comprising a workcell 310, sterilization system 320, fluid connector 330 and fluid devices 340. In some embodiments, the fluid devices 340 may comprise a main feedthrough and a fluid device (e.g., reagent) feedthrough. The sterilization system 320 may comprise a sterilant source 322, pump 324, and heater (e.g., desiccant/dryer) 326. For example, the heater 326 may be configured to aerate at a predetermined set of conditions. The sterilization system 320 may be coupled and in fluid communication with one or more of the workcell 310, fluid connector 330, and fluid device 340. In some embodiments, a robot (not shown) may be configured to manipulate and operate the cell processing system 300. For example, the fluid connector 330 may be coupled to one or more of the fluid devices 340 and instruments (not shown). One or more of the workcell 310, fluid connector 330, and fluid devices 340 may be sterilized and/or aerated by circulating one or more of a sterilant and fluid (e.g., heated air, vaporized hydrogen peroxide (VHP)) using the sterilization system 320. In some embodiments, the sterilization system 320 may comprise one or more of vaporized hydrogen peroxide (VHP), electron-beam (e-beam) sterilization, dry thermal decontamination, and steam-in-place. In some embodiments, the sterilization system 320 may provide a sterility assurance level (SAL) of at least 10-3 SAL.

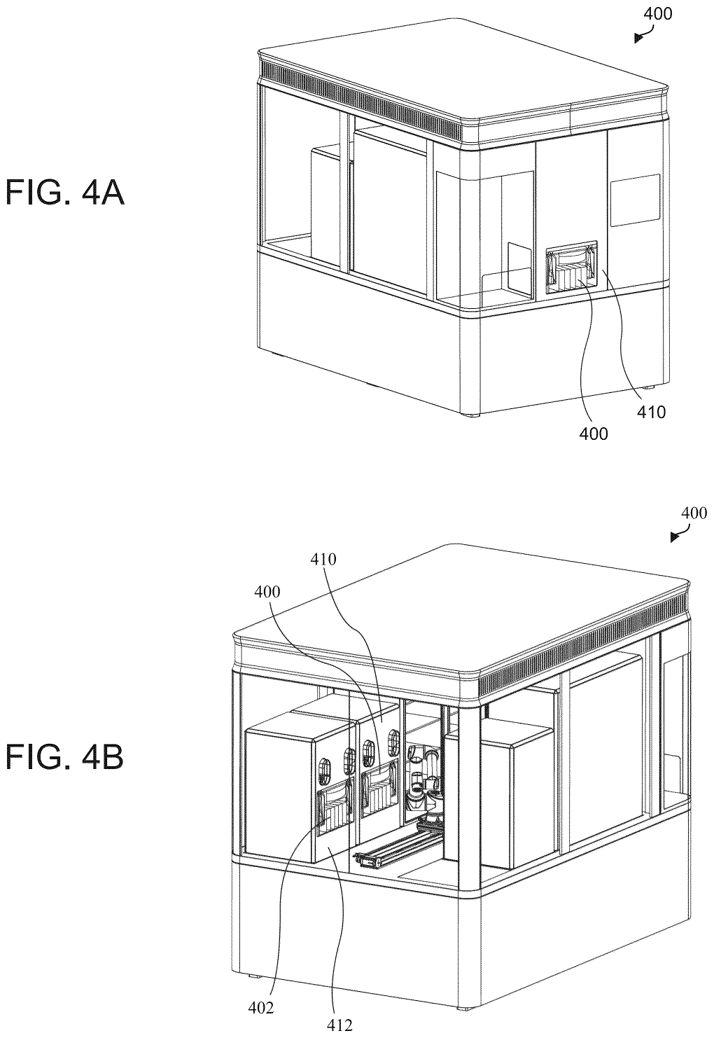

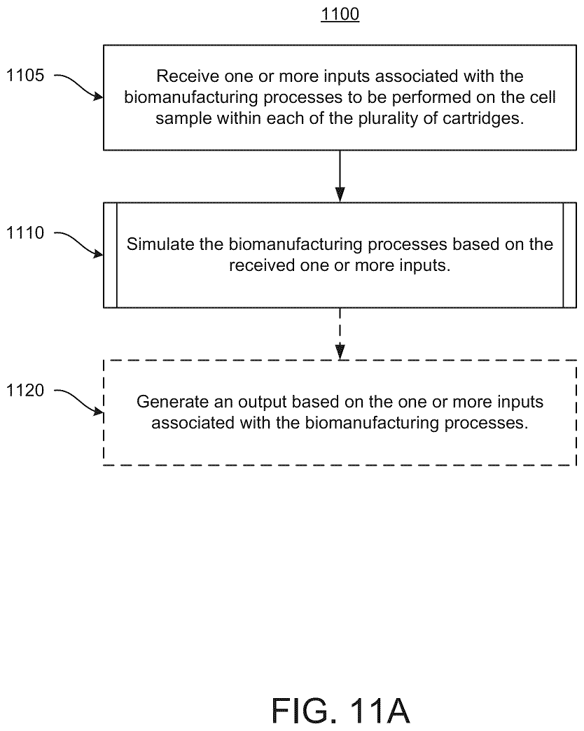

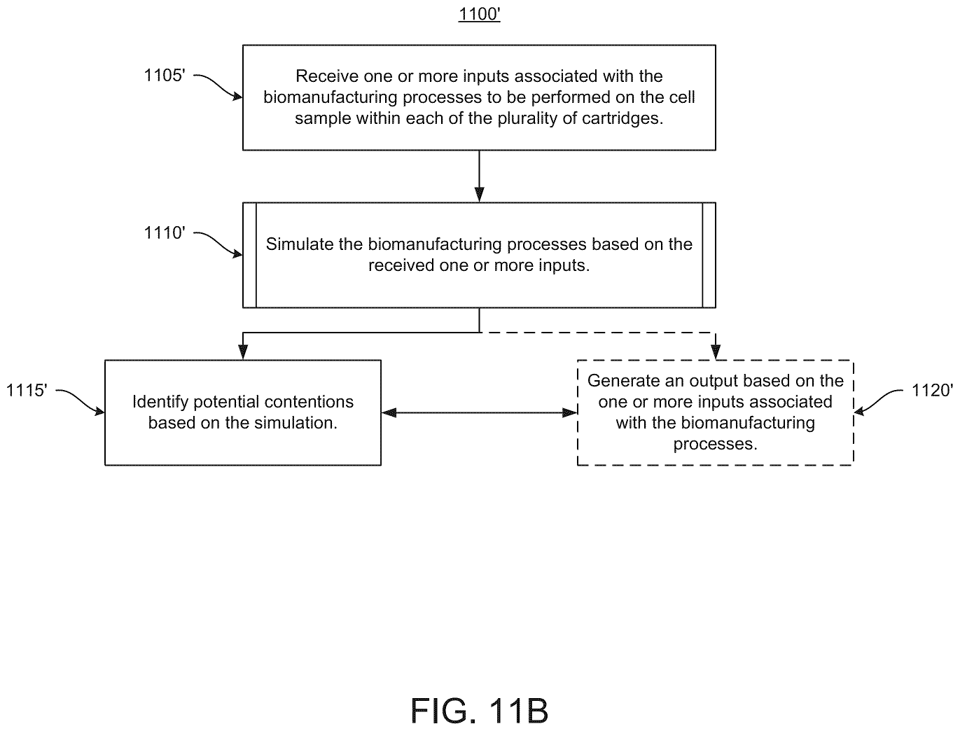

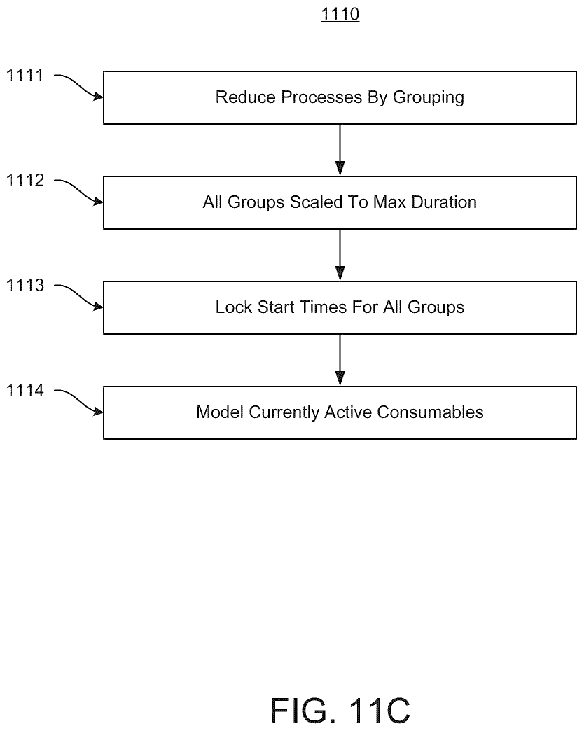

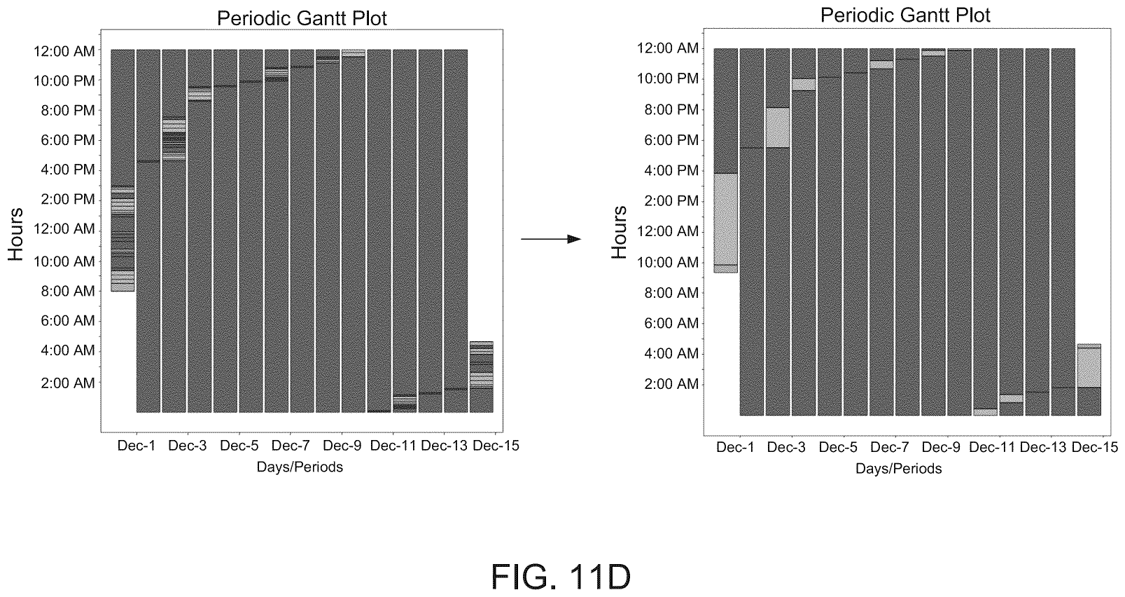

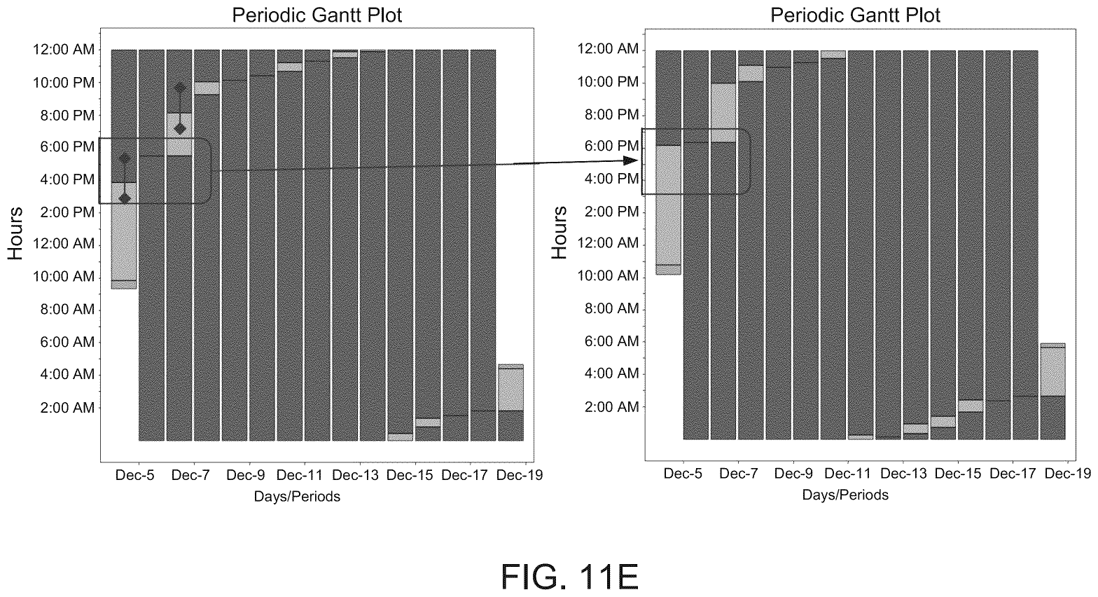

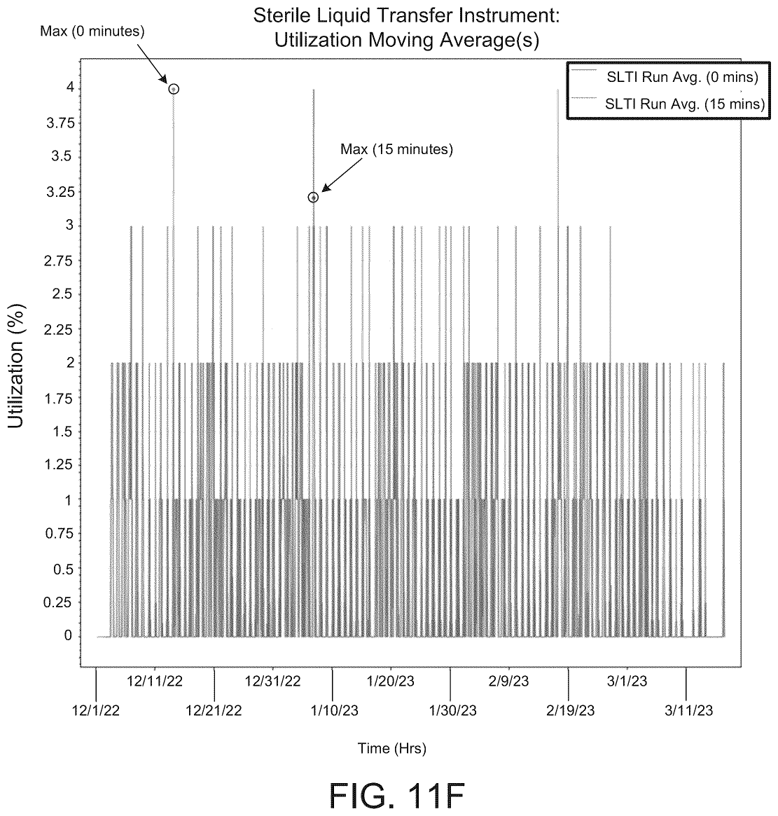

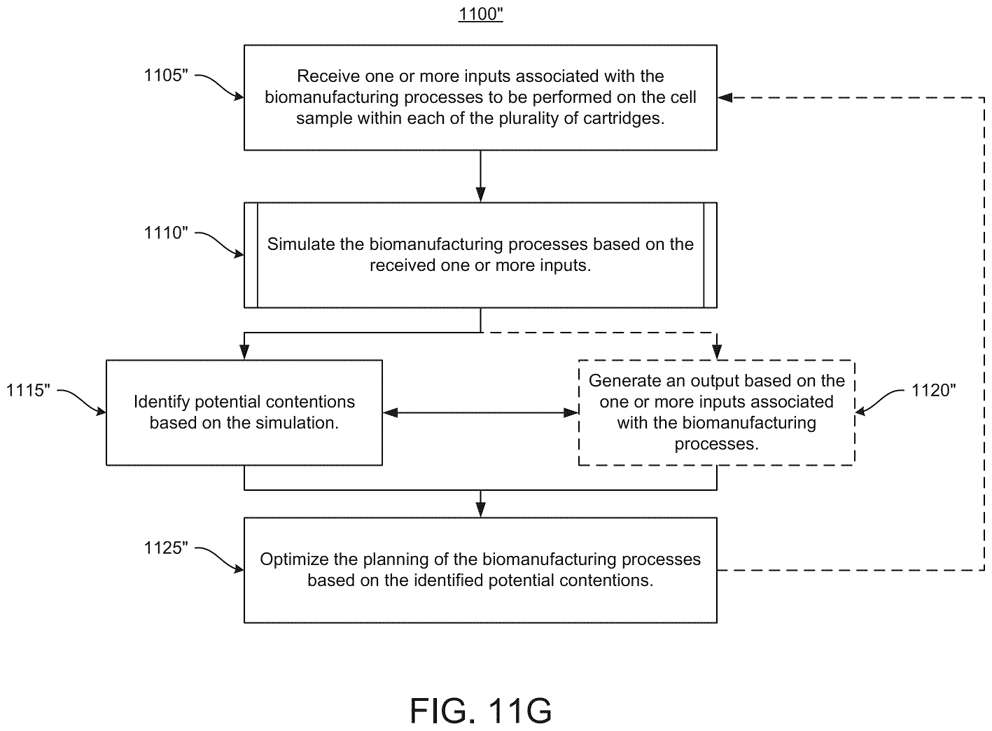

FIGS. 4A and 4B illustrate perspective views of a cell processing system 400 comprising a cartridge 400, 402, feedthrough 410, 412, and fluid connector 420, 422 (e.g., sterile liquid transfer instrument). For example, cartridge 400 is shown in the feedthrough 410 in FIG. 11A while a robot (not shown) has moved cartridge 400 to fluid connector 420.

i. Robot

Generally, a robot of the workcell may comprise any mechanical device capable of moving a cartridge from one location to another location. For example, the robot may comprise a mechanical manipulator (e.g., an arm) in a fixed location, or attached to a linear rail, or a 2- or 3-dimensional rail system. In an embodiment, the robot comprises a robotic shuffle system. In a further embodiment, the robot comprises a wheeled device. In some embodiments, the system comprises two or more robots of the same or different type (e.g., two robotic arms each independently configured for moving cartridges between instruments). The robot may also comprise an end effector for precise handling of different cartridges or barcode scanning or radio-frequency identification tag (RFID) reading.

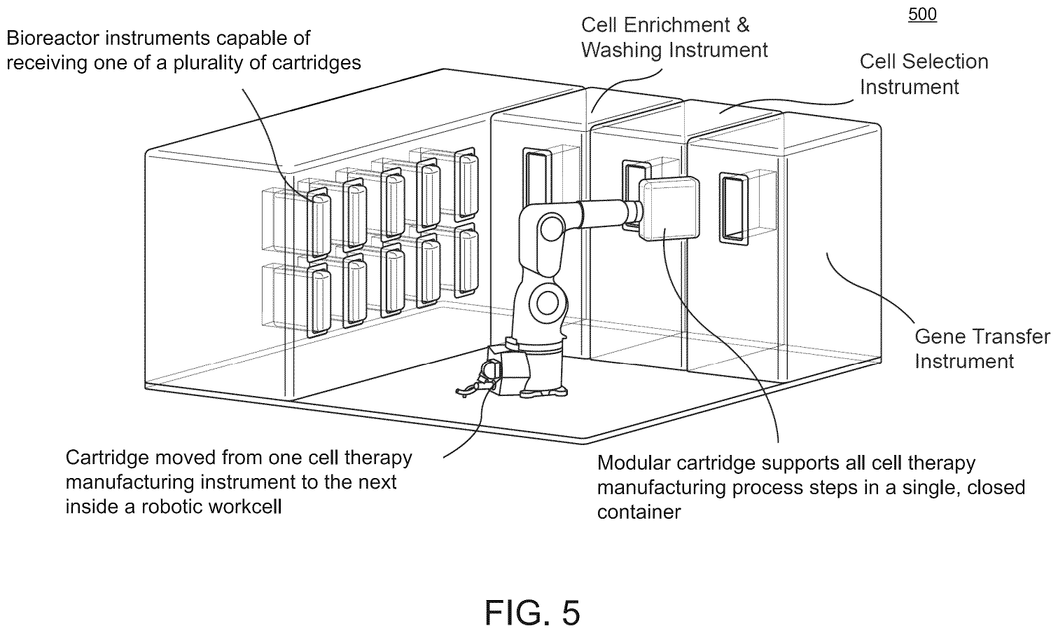

FIG. 5 is a perspective view of a cell processing system 500 in which a robot arm moves cartridges between slots in various instruments each configured to perform a different cell processing step. In some embodiments, the same cartridge can be received by different instruments. The system 500 may comprise a modular design to accommodate different instrument configurations. In some embodiments, a plurality of cartridges may be processed in parallel. Each cartridge may contain a cell product intended for different recipients. For example, a cell product from a single donor may be split between a plurality of cartridges to generate a predetermined quantity of cell product for therapeutic use such as when a donor is providing product for several recipients (e.g., for allogeneic transplant). In some embodiments, the cell product for a single recipient may be split between a plurality of cartridges to generate a predetermined quantity of product for therapeutic use in that recipient. In some embodiments, the cell product for a single recipient may be split between a plurality of cartridges to generate a predetermined quantity of several cell products with unique genetic modifications, which may be recombined in certain ratios for therapeutic use in that recipient.

ii. Reagent Vault

In some embodiments, the workcell of the cell processing system comprises a reagent vault (or reagent vaults) where reagents are stored including but not limited to cell culture media, buffer, cytokines, proteins, enzymes, polynucleotides, transfection reagents, non-viral vectors, viral vectors, antibiotics, nutrients, cryoprotectants, solvents, cellular materials, and pharmaceutically acceptable excipients. Additionally, or alternatively, waste may be stored in the reagent vault. In some embodiments, in-process samples extracted from one or more cartridges may be stored in the reagent vault. The reagent vault may comprise one or more controlled temperature compartments (e.g., freezers, coolers, water baths, warming chambers, or others, at e.g. about −80° C., about −20° C., about 4° C., about 25° C., about 30° C., about 37° C., and about 42° C.). Temperatures in these compartments may be varied during the cell manufacturing process to heat or cool reagents. In embodiments of the methods of the disclosure, a cartridge may be moved by the robot (or manually by an operator) to the reagent vault. The reagent vault interfaces with one or more sterile liquid transfer ports on the cartridge, and the reagent or material is dispensed into the cartridge. Optionally, fluid is added or removed from the cartridge before, during, or after reagent addition or removal. In some embodiments, the system comprises a sterile liquid transfer instrument, similarly configured to transfer fluid into or out of the cartridge in an automated, manual, or semi-automated fashion. An operator may stock the sterile liquid transfer station with reagents manually, or they may be supplied by a robot (e.g. from a feedthrough or other location). In some cases, a robot moves a reagent or reagents from the reagent vault to the sterile liquid transfer station. The reagent vault may have automated doors to permit access by the robot for sterile liquid transfer devices and/or other reagent vessels, optionally each under independent closed loop temperature control. The devices and vessels may be configured for pick-and-place movement by the robot. In some embodiments, the reagent vault may comprise one or more sample pickup areas. For example, a robot may be configured to move one or more reagents to and from one or more of the sample pickup areas.

iii. Controller

In embodiments, a cell processing system 100 may comprise a controller 120 (e.g., computing device) comprising one or more of a processor 122, memory 124, communication device, 126, input device 128, and display 130. The controller 120 may be configured to control (e.g., operate) the workcell 110. The controller 120 may comprise a plurality of devices. For example, the workcell 110 may enclose one or more components of the controller 120 (e.g., processor 122, memory 124, communication device 126) while one or more components of the controller 120 may be provided remotely to the workcell 110 (e.g., input device 128, display 130).

iv. Processor

The processor (e.g., processor 122) described here may process data and/or other signals to control one or more components of the system. The processor may be configured to receive, process, compile, compute, store, access, read, write, and/or transmit data and/or other signals. Additionally, or alternatively, the processor may be configured to control one or more components of a device (e.g., console, touchscreen, personal computer, laptop, tablet, server).

In some embodiments, the processor may be configured to access or receive data and/or other signals from one or more of workcell 110, server, controller 120, and a storage medium (e.g., memory, flash drive, memory card, database). In some embodiments, the processor may be any suitable processing device configured to run and/or execute a set of instructions or code and may include one or more data processors, image processors, graphics processing units (GPU), physics processing units, digital signal processors (DSP), analog signal processors, mixed-signal processors, machine learning processors, deep learning processors, finite state machines (FSM), compression processors (e.g., data compression to reduce data rate and/or memory requirements), encryption processors (e.g., for secure wireless data transfer), and/or central processing units (CPU). The processor may be, for example, a general-purpose processor, Field Programmable Gate Array (FPGA), an Application Specific Integrated Circuit (ASIC), a processor board, and/or the like. The processor may be configured to run and/or execute application processes and/or other modules, processes and/or functions associated with the system. The underlying device technologies may be provided in a variety of component types (e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, and the like.

The systems, devices, and/or methods described herein may be performed by software (executed on hardware), hardware, or a combination thereof. Hardware modules may include, for example, a general-purpose processor (or microprocessor or microcontroller), a field programmable gate array (FPGA), and/or an application specific integrated circuit (ASIC). Software modules (executed on hardware) may be expressed in a variety of software languages (e.g., computer code), including structured text, typescript, C, C++, C #, Java®, Python, Ruby, Visual Basic®, and/or other object-oriented, procedural, or other programming language and development tools. Examples of computer code include, but are not limited to, micro-code or micro-instructions, machine instructions, such as produced by a compiler, code used to produce a web service, and files containing higher-level instructions that are executed by a computer using an interpreter. Additional examples of computer code include, but are not limited to, control signals, encrypted code, and compressed code.

v. Memory

The cell processing systems and devices described here may include a memory (e.g., memory 124) configured to store data and/or information. In some embodiments, the memory may include one or more of a random-access memory (RAM), static RAM (SRAM), dynamic RAM (DRAM), a memory buffer, an erasable programmable read-only memory (EPROM), an electrically erasable read-only memory (EEPROM), a read-only memory (ROM), flash memory, volatile memory, non-volatile memory, combinations thereof, and the like. In some embodiments, the memory may store instructions to cause the processor to execute modules, processes, and/or functions associated with the device, such as image processing, image display, sensor data, data and/or signal transmission, data and/or signal reception, and/or communication. Some embodiments described herein may relate to a computer storage product with a non-transitory computer-readable medium (also may be referred to as a non-transitory processor-readable medium) having instructions or computer code thereon for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The computer code (also may be referred to as code or algorithm) may be those designed and constructed for the specific purpose or purposes. In some embodiments, the memory may be configured to store any received data and/or data generated by the controller and/or workcell. In some embodiments, the memory may be configured to store data temporarily or permanently.

vi. Input Device

In some embodiments, the display may include and/or be operatively coupled to an input device 128 (e.g., touch screen) configured to receive input data from a user. For example, user input to an input device 128 (e.g., keyboard, buttons, touch screen) may be received and processed by a processor (e.g., processor 122) and memory (e.g., memory 124) of the system 100. The input device may include at least one switch configured to generate a user input. For example, an input device may include a touch surface for a user to provide input (e.g., finger contact to the touch surface) corresponding to a user input. An input device including a touch surface may be configured to detect contact and movement on the touch surface using any of a plurality of touch sensitivity technologies including capacitive, resistive, infrared, optical imaging, dispersive signal, acoustic pulse recognition, and surface acoustic wave technologies. In embodiments of an input device including at least one switch, a switch may have, for example, at least one of a button (e.g., hard key, soft key), touch surface, keyboard, analog stick (e.g., joystick), directional pad, mouse, trackball, jog dial, step switch, rocker switch, pointer device (e.g., stylus), motion sensor, image sensor, and microphone. A motion sensor may receive user movement data from an optical sensor and classify a user gesture as a user input. A microphone may receive audio data and recognize a user voice as a user input.

In some embodiments, the cell processing system may optionally include one or more output devices in addition to the display, such as, for example, an audio device and haptic device. An audio device may audibly output any system data, alarms, and/or notifications. For example, the audio device may output an audible alarm when a malfunction is detected. In some embodiments, an audio device may include at least one of a speaker, a piezoelectric audio device, a magnetostrictive speaker, and/or a digital speaker. In some embodiments, a user may communicate with other users using the audio device and a communication channel. For example, a user may form an audio communication channel (e.g., VoIP call).

Additionally, or alternatively, the system may include a haptic device configured to provide additional sensory output (e.g., force feedback) to the user. For example, a haptic device may generate a tactile response (e.g., vibration) to confirm user input to an input device (e.g., touch surface). As another example, haptic feedback may notify that user input is overridden by the processor.

vii. Communication Device

In some embodiments, the controller may include a communication device (e.g., communication device 126) configured to communicate with another controller and one or more databases. The communication device may be configured to connect the controller to another system (e.g., Internet, remote server, database, workcell) by wired or wireless connection. In some embodiments, the system may be in communication with other devices via one or more wired and/or wireless networks. In some embodiments, the communication device may include a radiofrequency receiver, transmitter, and/or optical (e.g., infrared) receiver and transmitter configured to communicate with one or more devices and/or networks. The communication device may communicate by wires and/or wirelessly.

The communication device may include RF circuitry configured to receive and send RF signals. The RF circuitry may convert electrical signals to/from electromagnetic signals and communicate with communications networks and other communications devices via the electromagnetic signals. The RF circuitry may include well-known circuitry for performing these functions, including but not limited to an antenna system, an RF transceiver, one or more amplifiers, a tuner, one or more oscillators, a digital signal processor, a CODEC chipset, a subscriber identity module (SIM) card, memory, and so forth.

Wireless communication through any of the devices may use any of plurality of communication standards, protocols and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), EtherCAT, OPC Unified Architecture, or any other suitable communication protocol. In some embodiments, the devices herein may directly communicate with each other without transmitting data through a network (e.g., through NFC, Bluetooth, WiFi, RFID, and the like).

In some embodiments, the systems, devices, and methods described herein may be in communication with other wireless devices via, for example, one or more networks, each of which may be any type of network (e.g., wired network, wireless network). The communication may or may not be encrypted. A wireless network may refer to any type of digital network that is not connected by cables of any kind. Examples of wireless communication in a wireless network include, but are not limited to cellular, radio, satellite, and microwave communication. However, a wireless network may connect to a wired network in order to interface with the Internet, other carrier voice and data networks, business networks, and personal networks. A wired network is typically carried over copper twisted pair, coaxial cable and/or fiber optic cables. There are many different types of wired networks including wide area networks (WAN), metropolitan area networks (MAN), local area networks (LAN), Internet area networks (IAN), campus area networks (CAN), global area networks (GAN), like the Internet, and virtual private networks (VPN). Hereinafter, network refers to any combination of wireless, wired, public and private data networks that are typically interconnected through the Internet, to provide a unified networking and information access system. Cellular communication may encompass technologies such as GSM, PCS, CDMA or GPRS, W-CDMA, EDGE or CDMA2000, LTE, WiMAX, and 5G networking standards. Some wireless network deployments combine networks from multiple cellular networks or use a mix of cellular, Wi-Fi, and satellite communication.

viii. Display

Image data may be output on a display e.g., display 130) of a cell processing system. In some embodiments, a display may include at least one of a light emitting diode (LED), liquid crystal display (LCD), electroluminescent display (ELD), plasma display panel (PDP), thin film transistor (TFT), organic light emitting diodes (OLED), electronic paper/e-ink display, laser display, and/or holographic display.

ix. Graphical User Interface

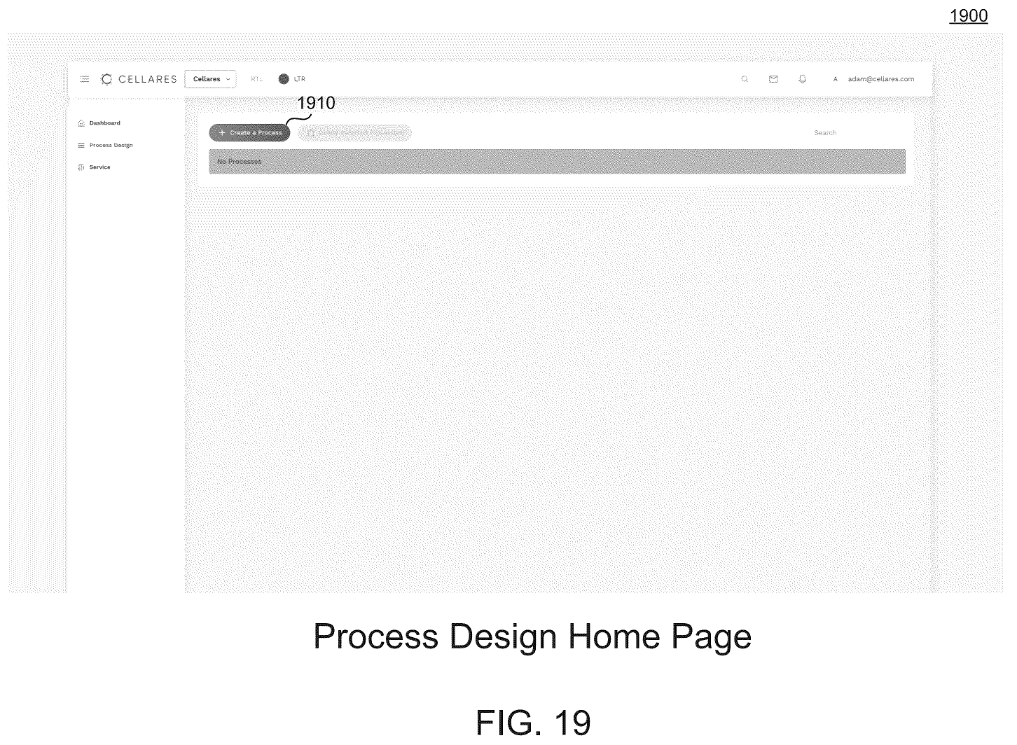

In some embodiments, as indicated above, a GUI may be configured for designing a process and monitoring a product. FIG. 19 is an embodiment of a GUI 1900 comprising an initial process design interface. For example, GUI 1900 may be a process design home page. The GUI 1900 may indicate that no processes have been selected or loaded. A create icon 1910 (e.g., “Create a Process”) may be selectable for a user to begin a process design process. In some embodiments, one or more of the GUIs described herein may include a search bar.

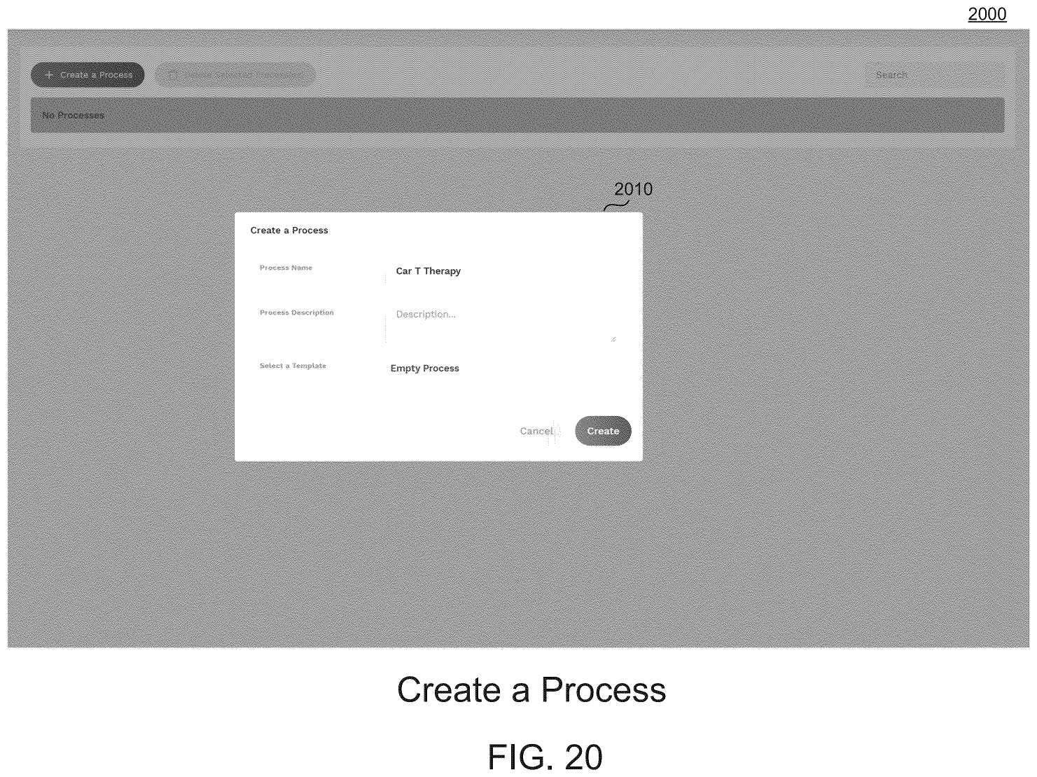

FIG. 20 is an embodiment of a GUI 2000 comprising a display related to a process that has not yet initiated, or an empty process. GUI 2000 may be displayed following selection of the create icon 1910 in FIG. 19. For example, GUI 2000 may comprise a process creation window 2010 allowing a user to input and/or select one or more of a process name, process description, and template. In some embodiments, a user may select from a list of predetermined templates. For example, a user may create a process and save it as a template for later selection.

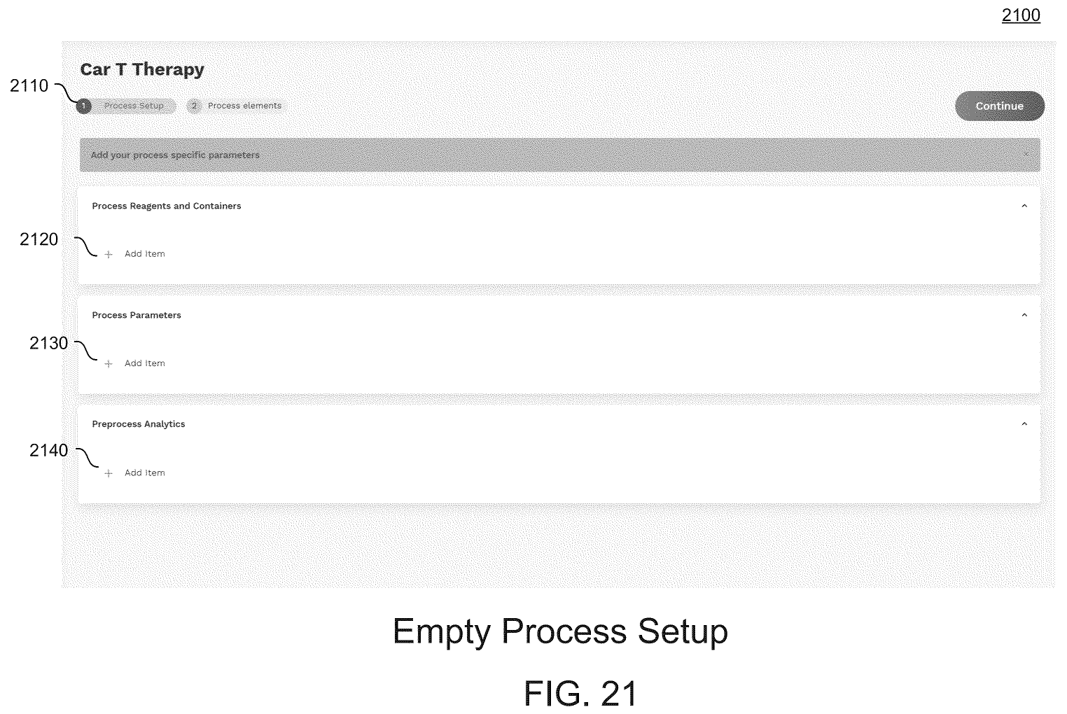

FIG. 21 is an embodiment of a GUI 2100 comprising relating to an empty process. GUI 2100 may be displayed following confirmation in GUI 2100 that a process is to be created. GUI 2100 may indicate the process name (e.g., Car T Therapy) and may highlight Process Setup icon 2110 and allow process specific parameters to be added such as process reagents and containers, process parameters, and preprocess analytics. GUI 2100 may further comprise an Add Process Reagents and Containers icon 2120, Add Process Parameters icon 2130, and Add Preprocess Analytics icon 2140. Once process setup is completed, one or more process elements may be specified.

In some embodiments, the GUI 2100 may comprise one or more predetermined templates for a set of biological processes (e.g., CAR-T, NK cells, HSC, TIL, etc.). For example, the templates may aid process development and be validated starting points for process development. The templates may be further modified (e.g., customized) based on user requirements.

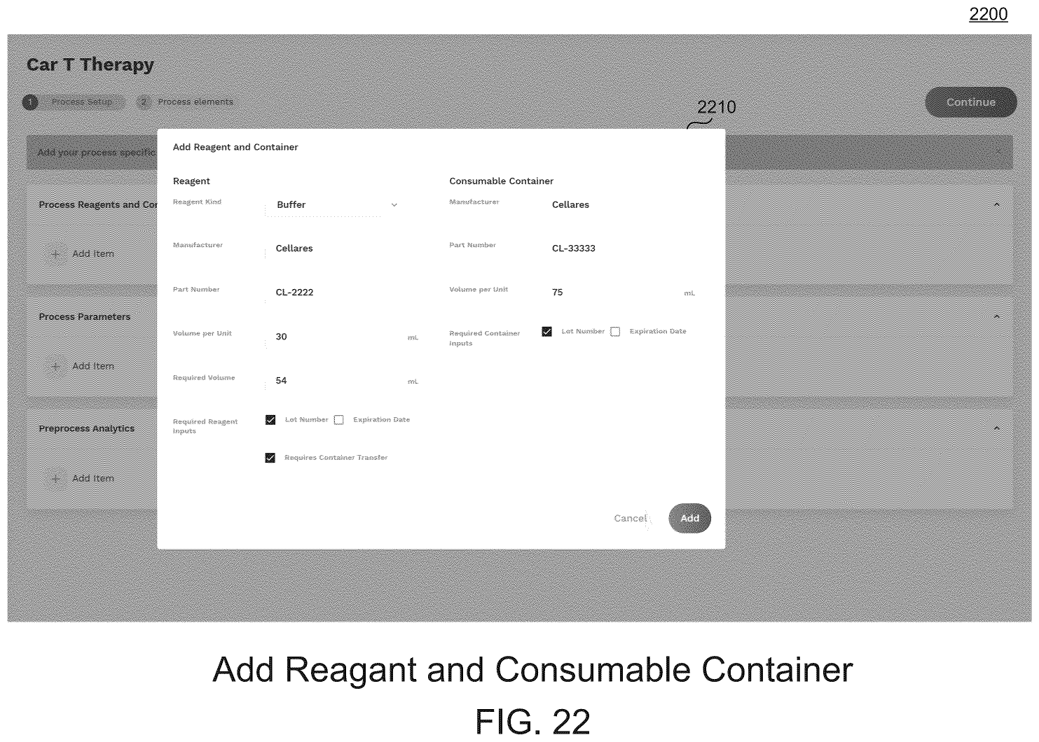

FIG. 22 is an embodiment of a GUI 2200 comprising a display relating to adding a reagent and a container. GUI 2200 may be displayed following selection of an Add Process Reagents and Containers icon 2020 in FIG. 20. For example, GUI 2200 may comprise an Add Reagent and Container window 2210 enabling a user to input and/or select one or more reagents comprising a reagent kind, manufacturer, part number, volume per unit, required volume and required reagent inputs (e.g., lot number, expiration date, requires container transfer). Add Reagent and Container window 2210 may comprise one or more of an input field, selection box, drop-down selector, and the like. Furthermore, the Add Reagent and Container window 2010 may enable a user to input and/or select one or more containers comprising a manufacturer, part number, volume per unit, and required container inputs (e.g., lot number, expiration date). In some embodiments, a user may select from a list of predetermined templates. For example, a user may create a process and save it as a template.

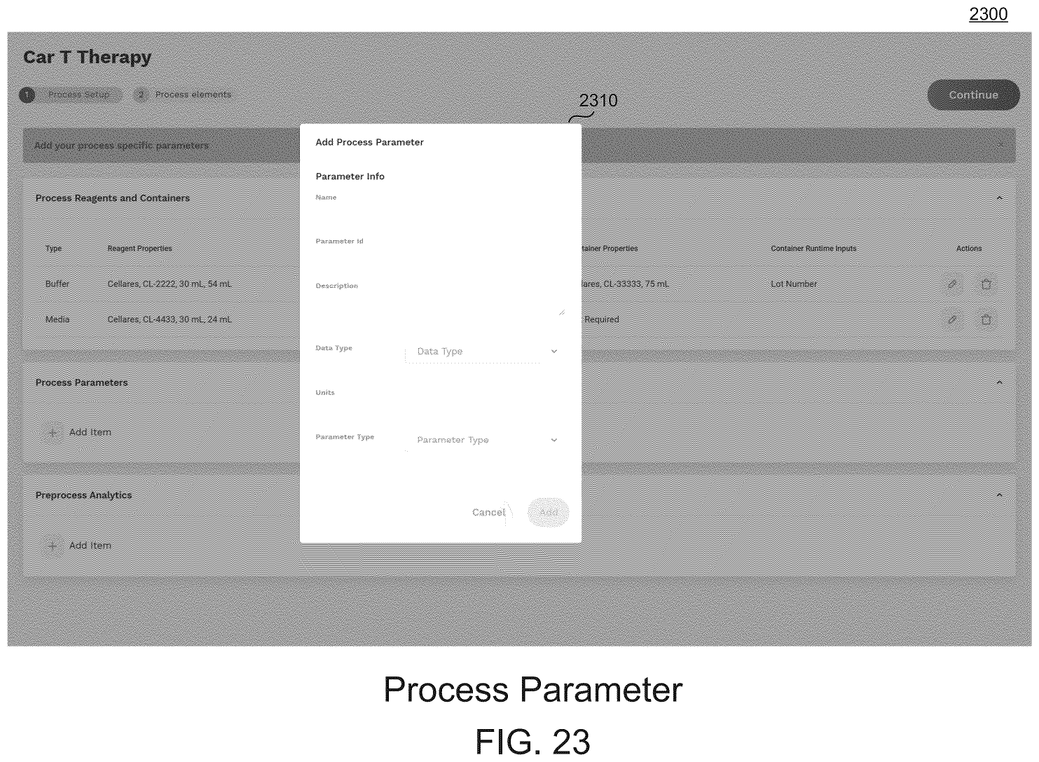

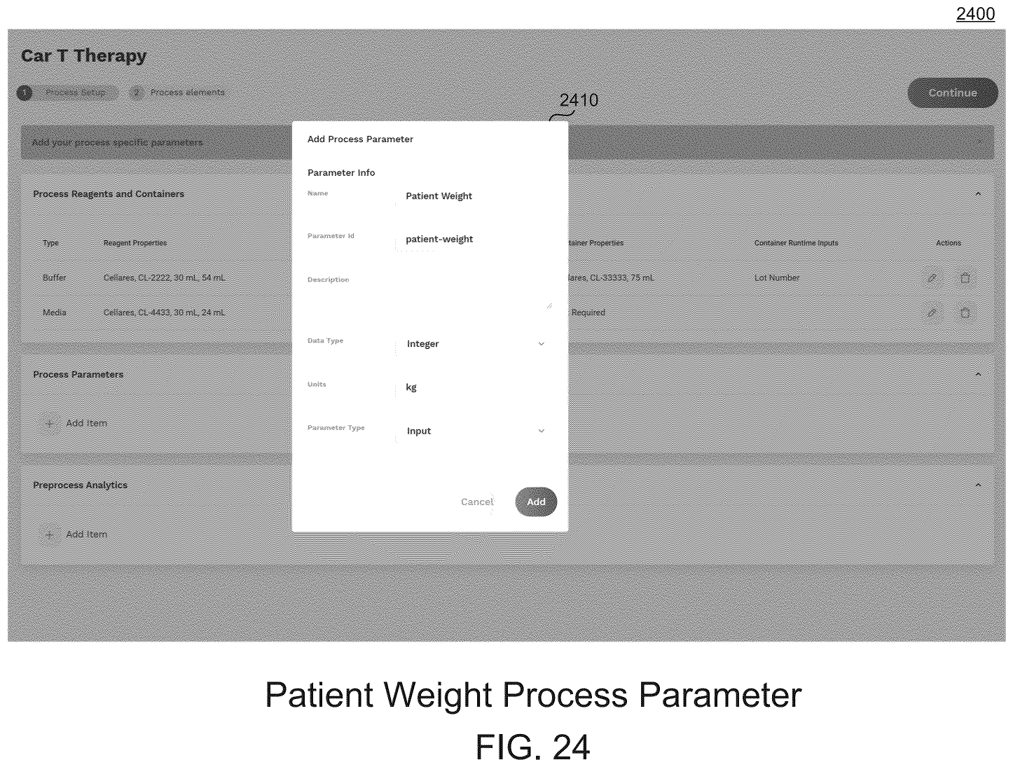

FIG. 23 is an embodiment of a GUI 2300 comprising a display relating to a process parameter. GUI 2300 may be displayed following selection of an Add Process Reagents and Containers icon 2030 in FIG. 20. For example, GUI 2300 may comprise an Add Process Parameter window 2310 enabling a user to input and/or select one or more parameters comprising a name, parameter identification, description, data type, units, and parameter type. Add Process Parameter window 2210 may comprise one or more of an input field, selection box, drop-down selector, and the like. In some embodiments, a user may select from a list of predetermined templates. For example, a user may create a parameter and save it as a template. FIG. 24 is an embodiment of a GUI 2400 comprising a display relating to a patient weight process parameter. For example, GUI 2400 may comprise an Add Process Parameter window 2310 having filled in parameter information including patient weight, data type (e.g., integer), units (e.g., kg), and parameter type (e.g., input).

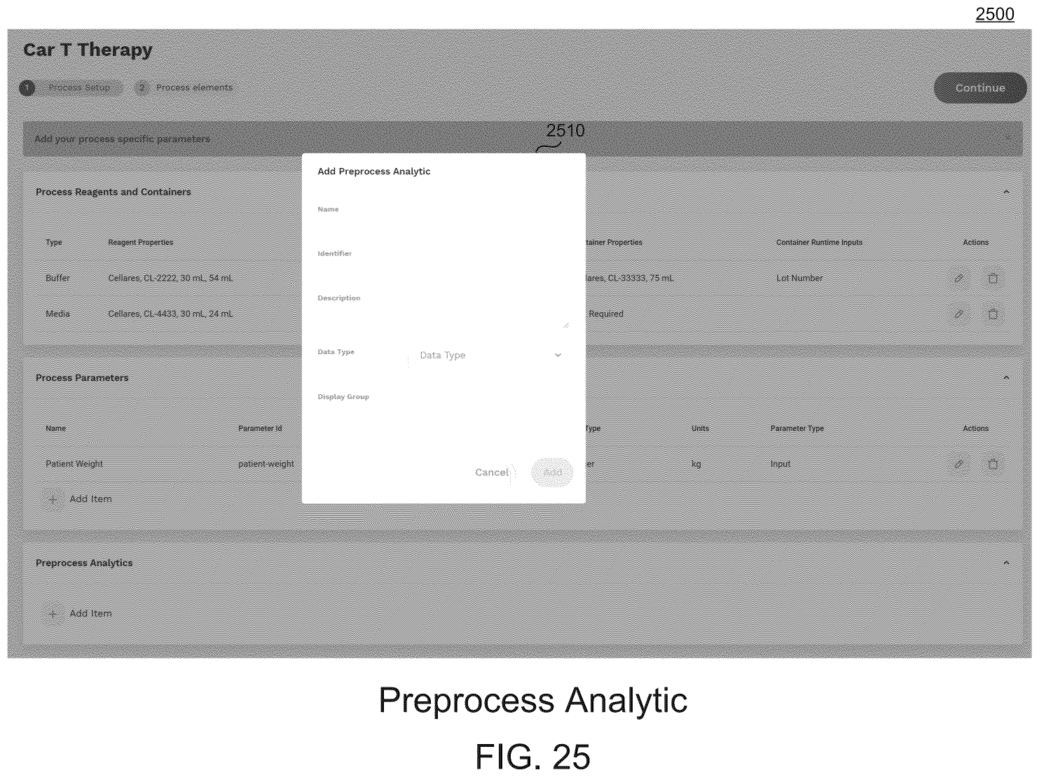

FIG. 25 is an embodiment of a GUI 2500 relating to a preprocess analytic. GUI 2500 may be displayed following selection of an Add Preprocess analytics icon 2040 in FIG. 20. For example, GUI 2500 may comprise an Add Preprocess Analytic window 2510 enabling a user to input and/or select one or more parameters comprising a name, identifier, description, data type, and display group. Add Preprocess Analytic window 2510 may comprise one or more of an input field, selection box, drop-down selector, and the like. In some embodiments, a user may select from a list of predetermined templates. For example, a user may create a parameter and save it as a template.

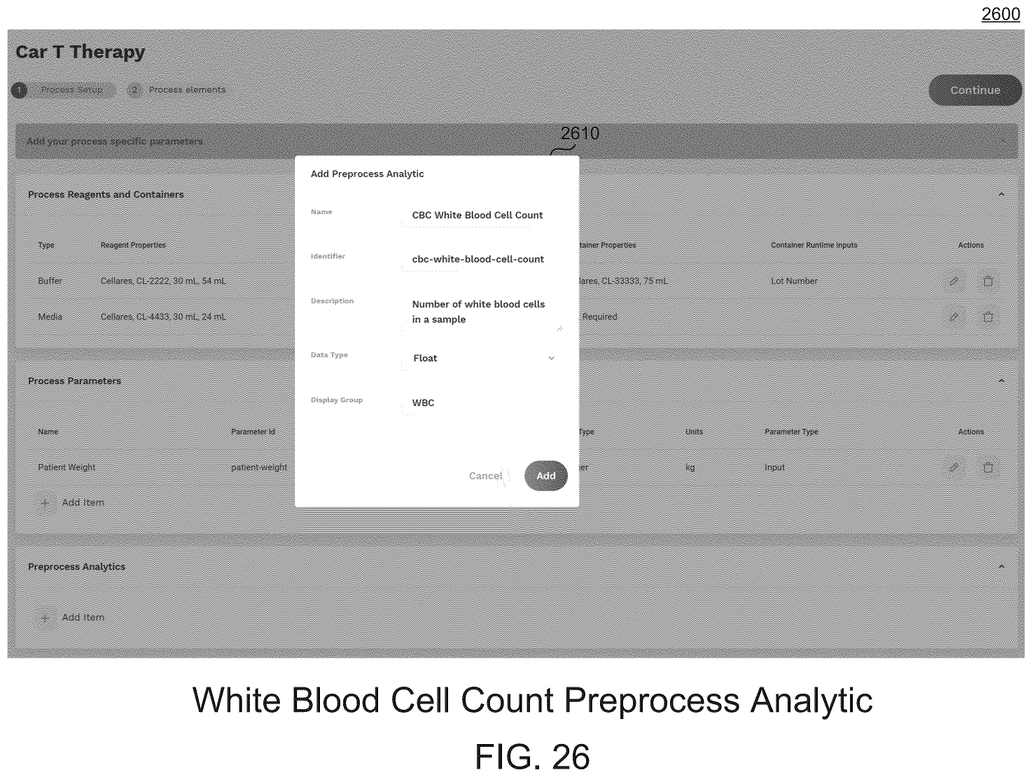

FIG. 26 is an embodiment of a GUI 2600 relating to a white blood cell count preprocess analytic. For example, GUI 2600 may comprise an Add Preprocess Analytic window 2610 having filled in preprocess analytic information including name (e.g., CBC White Blood Cell Count), identifier (e.g., CBC-white-blood-cell-count), description (e.g., Number of white blood cells in a sample), data type (e.g., float), and display group (e.g., WBC).

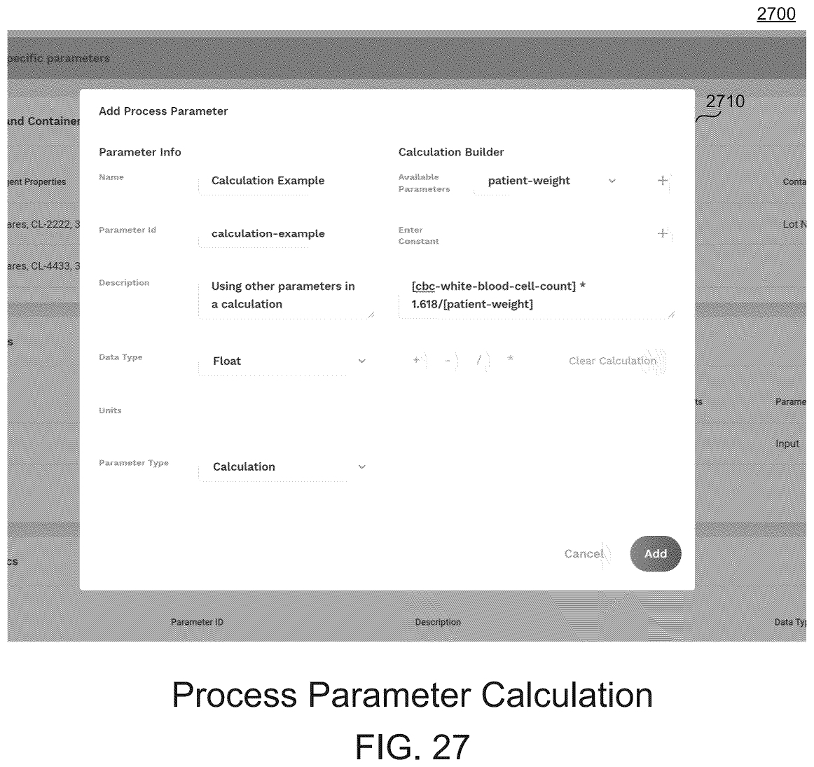

FIG. 27 is an embodiment of a GUI 2700 relating to a process parameter calculation. GUI 2700 may be displayed following selection of an Add Preprocess analytics icon 2040 in FIG. 20 and selection of a “Calculation” parameter type. For example, GUI 2700 may comprise an Add Preprocess Analytic window 2710 enabling a user to input and/or select one or more parameters comprising a name, identifier, description, data type, display group, units, and parameter type. Furthermore, a Calculation Builder may enable a user to define a formula (e.g., algorithm, equation) to perform a predetermined calculation. For example, a Calculation Builder may comprise one or more of a set of available parameters (e.g., patient weight), constant value, equation, and operands.

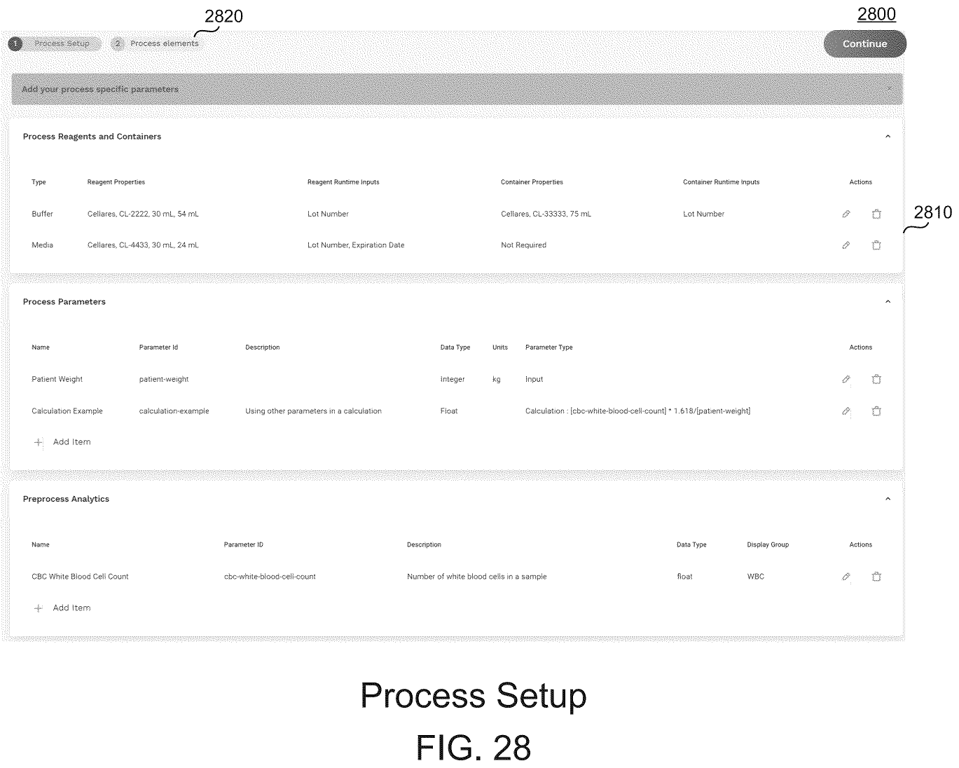

FIG. 28 is an embodiment of a GUI 2800 relating to a completed process setup. For example, GUI 2800 may comprise a Process Setup window 2810 having a filled in process reagents, containers, process parameters, and preprocess analytics. Once process setup is completed, one or more process elements may be specified.

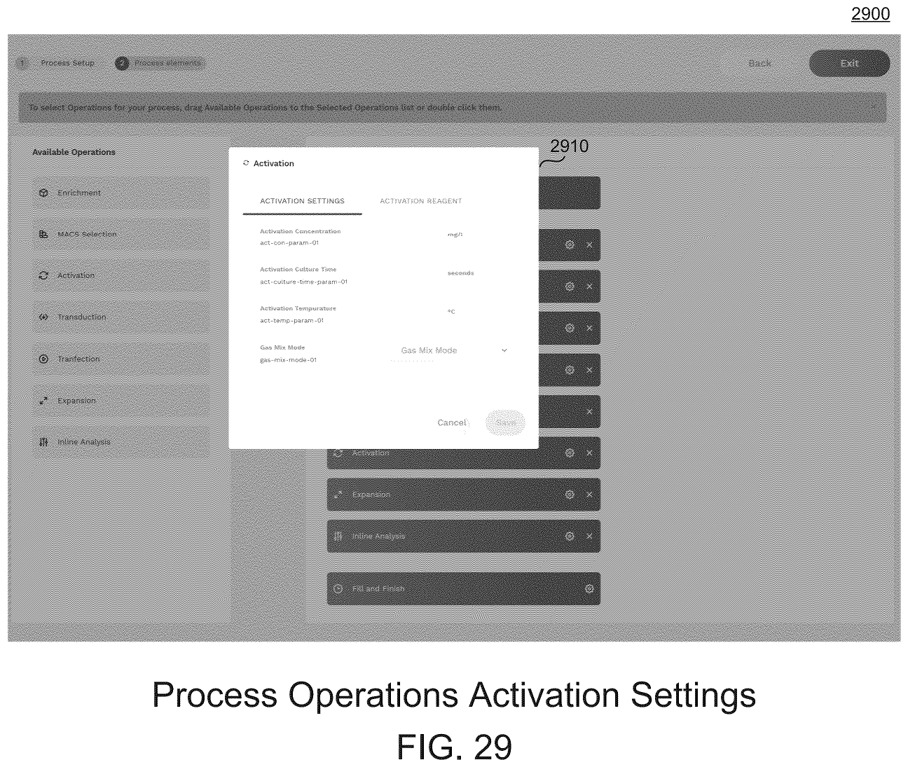

FIG. 29 is an embodiment of a GUI 2900 relating to process operations activation settings. GUI 2900 may be displayed following selection of a Process elements icon 2820 in FIG. 28. For example, GUI 2900 may comprise an Activation settings window 2910 allowing a user to input and/or select one or more of activation concentration (e.g., mg/L), activation culture time (e.g., seconds), activation temperature (e.g., ° C.), and gas mix mode. In some embodiments, a user may select from a list of predetermined templates. For example, a user may create a set of activation settings and save it as a template for later selection.

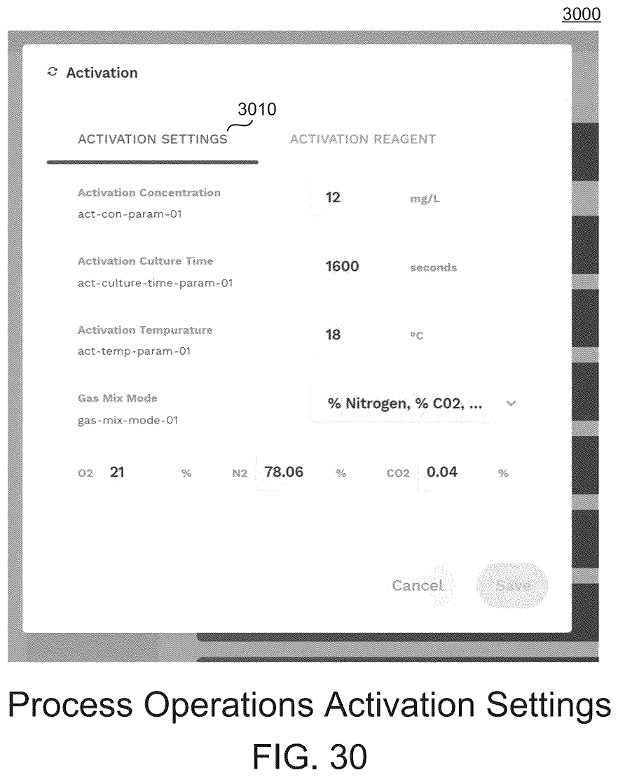

FIG. 30 is an embodiment of a GUI 3000 relating to a filled process operations activation settings. For example, GUI 3000 may comprise an Activation settings window 3010 having filled in Activation setting information. In some embodiments, a set of gases (e.g., O₂, N₂, CO₂) and corresponding concentrations may be specified.

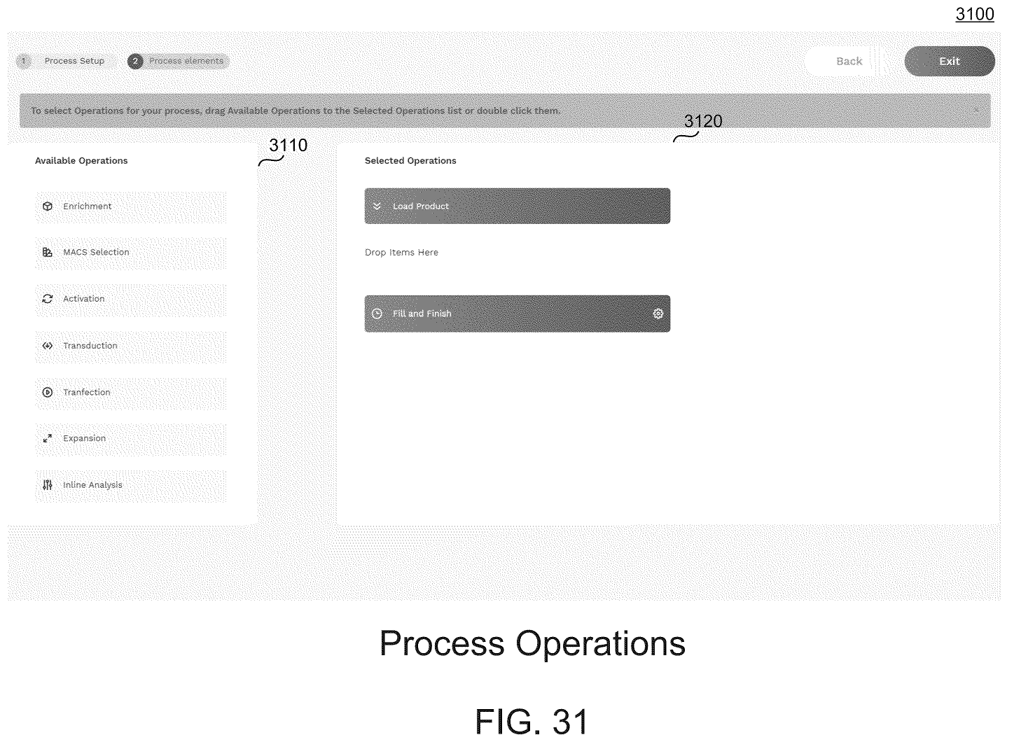

FIG. 31 is an embodiment of a GUI 3100 relating to a process operations interface. The GUI 3100 may comprise an Available Operations window 3110 and a Selected Operations window 3120. The available options for selection may include one or more biologic process inputs as described herein including, but not limited to, enrichment, MACS selection, activation, transduction, transfection, expansion, and inline analysis. One or more of the operations may be selected and dragged into the Selected Operations window 3120. The selected operations may be reordered within the Selected Operations window 3120.

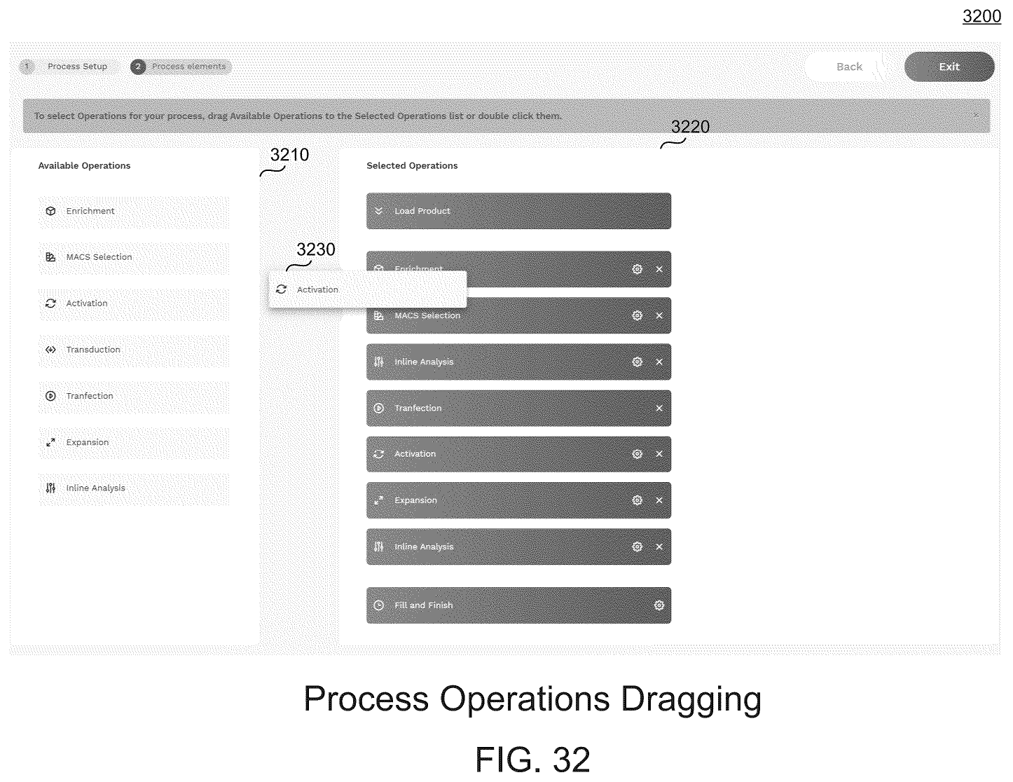

FIG. 32 is an embodiment of a GUI 3200 relating to dragging process operations. The GUI 3200 may comprise an Available Operations window 3210, a Selected Operations window 3220, and a selected (e.g., dragged) operation 3230 that may be drag and dropped between the Available Operations window 3210 and the Selected Operations window 3220. The Selected Operations window 3220 may comprise a plurality of selected operations.

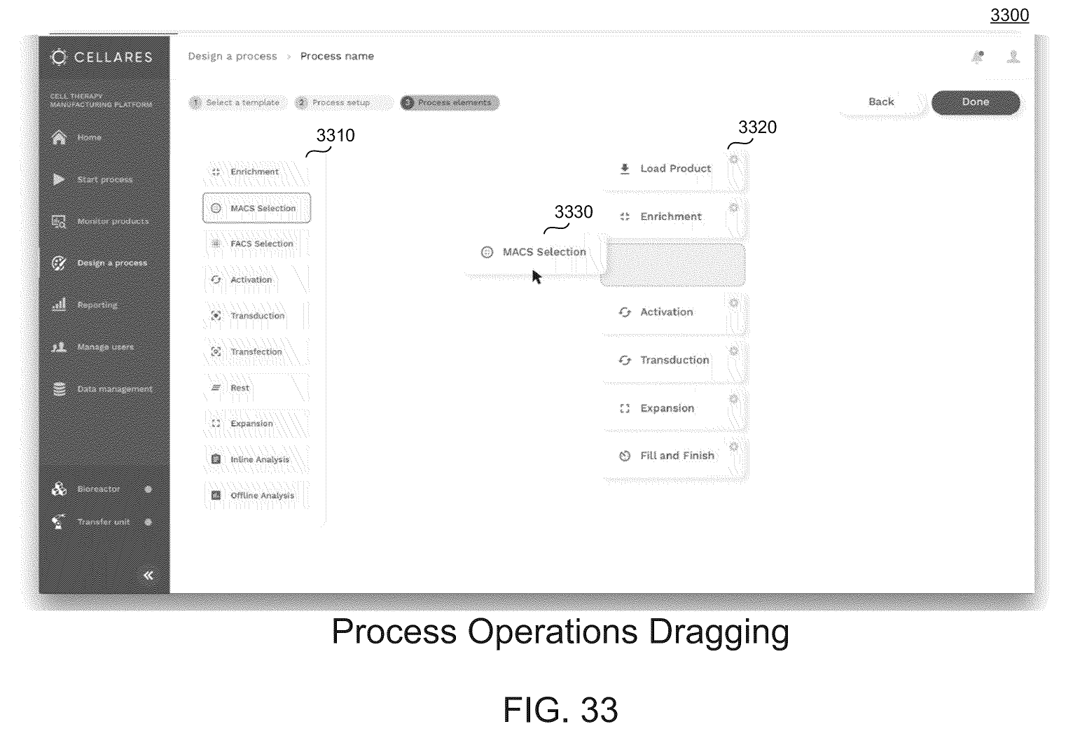

FIG. 33 is an embodiment of a GUI 3300 relating to dragging process operations. The GUI 3300 may comprise an Available Operations window 3310, a Selected Operations window 3320, and a selected (e.g., dragged) operation 3330 that may be drag and dropped between the Available Operations window 3310 and the Selected Operations window 3320. The Selected Operations window 3320 may comprise a plurality of selected operations.

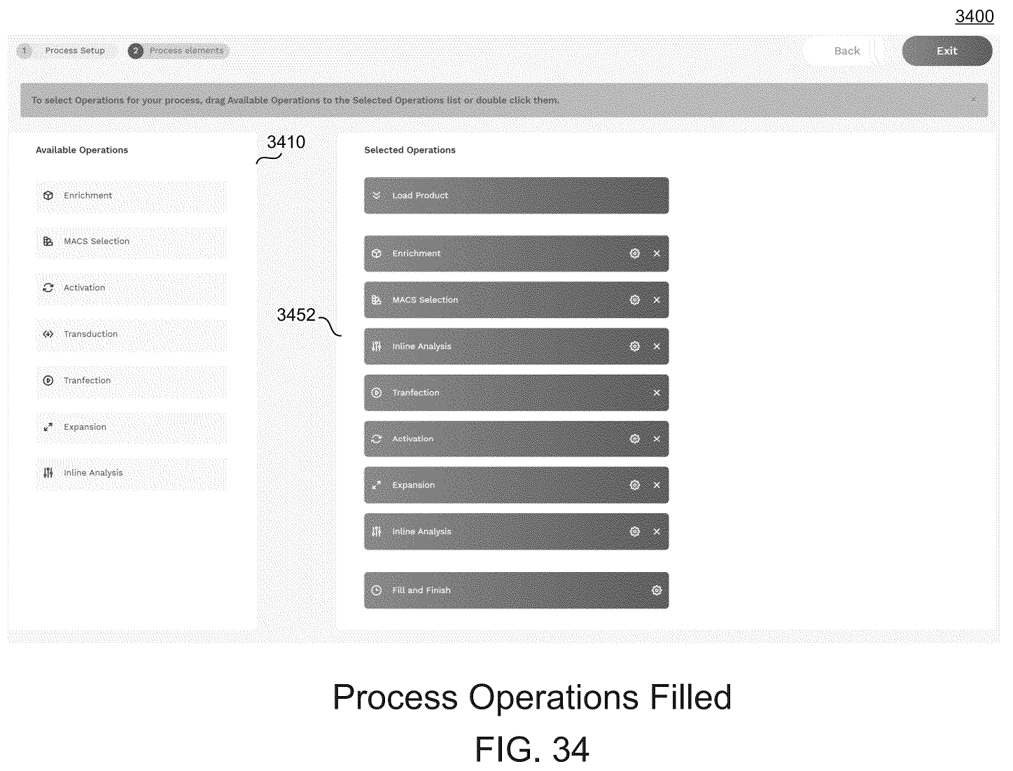

FIG. 34 is an embodiment of a GUI 3400 relating to a filled process operations. For example, the GUI 3400 may comprise an Available Operations window 3410 and a Selected Operations window 3420 comprising a completed set of selected operations. In some embodiments, the settings (e.g., parameters) of each operation may be selectively modified by the user by selecting a corresponding icon (e.g., gear icon).

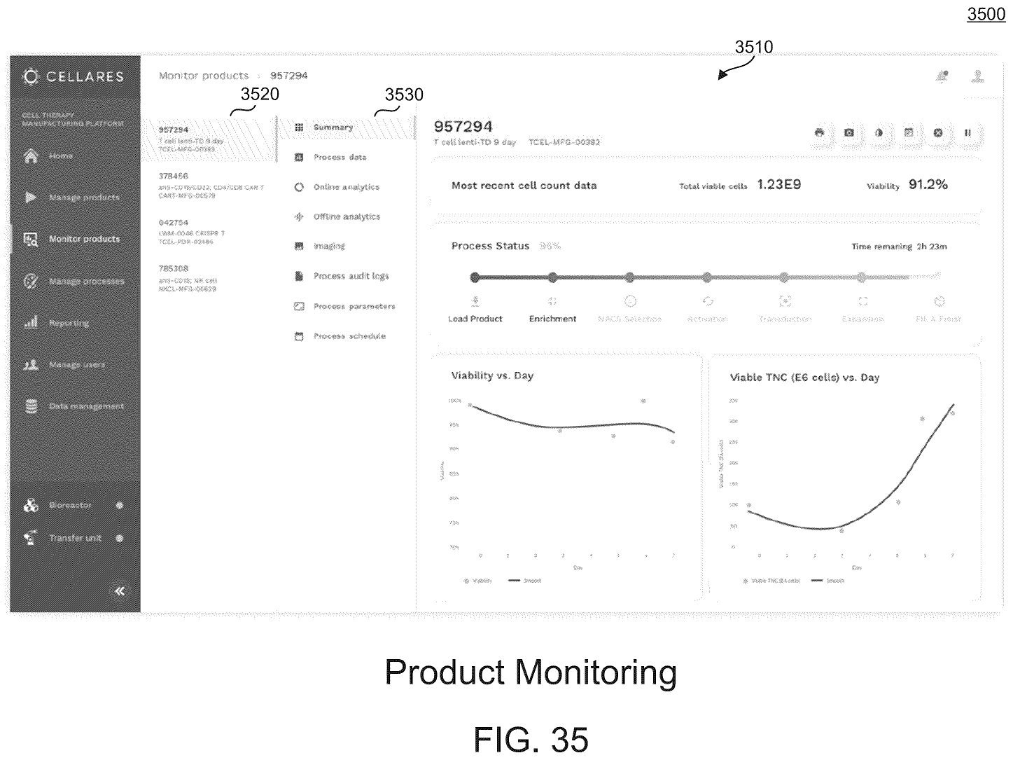

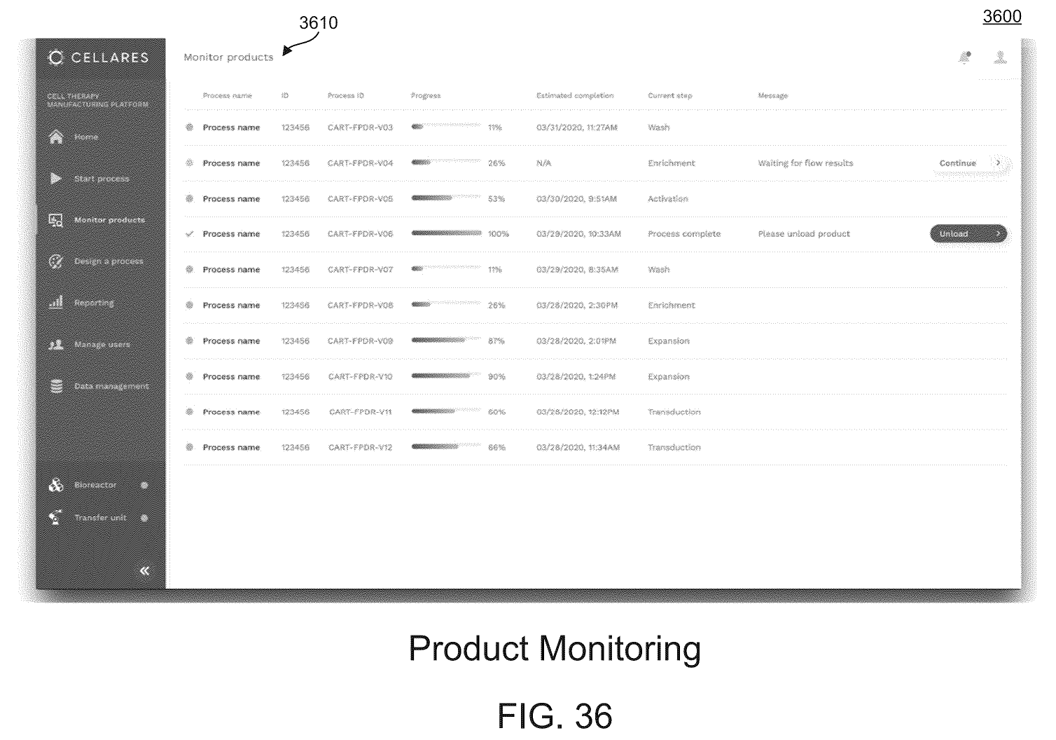

FIGS. 35 and 36 are embodiments of a GUI 3500 and 3600 relating to product monitoring. The GUI 3500 and 3600 may comprise respective monitoring windows 3510, 3610. For example, the GUI 3510 may monitor a plurality of products 3520 and output one or more product characteristics 3530 including, but not limited to, a summary, process data, online analytics, imaging, process audit logs, process parameters, and process schedule. The monitoring window 3610 may monitor one or more product characteristics of one or more products. For example, the product characteristics may include, but is not limited to, one or more of a process name, identification, process identification, progress, estimated completion, current step, and message.

### B. Cartridge

Generally, the cell processing systems described herein may comprise one or more cartridges including one or more modules configured to interface with an instrument or instruments. A robot (e.g., robotic arm) may be configured to move a cartridge and/or instrument to perform one or more cell processing steps. For example, a cartridge may comprise a bioreactor module and/or fluid connector (e.g., sterile liquid transfer port) coupled by the robot to a bioreactor instrument of a workcell. Once a predetermined processing step has been completed, the cartridge may be moved by the robot to another instrument of the workcell, and another cartridge may be coupled to the bioreactor instrument. Thus, a portable cartridge, in cooperation with shareable instruments of the workcell, may increase the efficiency, throughput, and flexibility of a cell manufacturing process. In some embodiments, the cartridge may optionally provide a self-contained device capable of performing one or more cell processing steps.

Various materials can be used to construct the cartridge and the cartridge housing, including metal, plastic, rubber, and/or glass, or combinations thereof. The cartridge, its components, and its housing may be molded, machined, extruded, 3D printed, or any combination thereof. The cartridge may contain components that are commercially available (e.g., tubing, valves, fittings)—these components may be attached or integrated with custom components or devices. The housing of the cartridge may constitute an additional layer of enclosure that further protects the sterility of the cell product. The operator may perform loading or unloading of the cartridge in an ISO 5 or better environment, utilizing aseptic technique to ensure that sterility of the contents of the cartridge is maintained when the cartridge is opened. In some embodiments, the operator may perform loading or unloading of the cartridge using manual aseptic connections (e.g., sterile tube welding). The robotic system may also perform sterile loading or unloading of liquids into and out of the cartridge through the use of the sterile liquid transfer instrument and sterile liquid transfer ports (of fluid connectors) on the cartridge.

In some embodiments, the modules may be integrated into a fixed configuration within the cartridge. Additionally, or alternatively, the modules may be configurable or moveable within the cartridge, permitting various cartridges to be assembled from shared modules. Similarly stated, the cartridge can be a single, closed unit with fixed components for each module; or the cartridge may contain configurable modules coupled by configurable fluidic, mechanical, optical, and electrical connections. In some embodiments, one or more sub-cartridges, each containing a set of modules, may be configured to be assembled to perform various cell processing workflows. The modules may each be provided in a distinct housing or may be integrated into a cartridge or sub-cartridge with other modules. The disclosure generally shows modules as distinct groups of components for the sake of simplicity but may be arranged in any suitable configuration. For example, the components for different modules may be interspersed with each other such that each module is defined by the set of connected components that collectively perform a predetermined function. However, the components of each module may or may not be physically grouped within the cartridge. In some embodiments, multiple cartridges may be used to process a single cell product through transfer of the cell product from one cartridge to another cartridge of the same or different type and/or by splitting cell product into more cartridges and/or pooling multiple cell products into fewer cartridges.

Generally, each of the instruments of the system interfaces with its respective module or modules on the cartridge e.g., an electroporation module on the cartridge (if present) is moved by the system to an electroporation instrument and interfaces with the electroporation instrument to perform an electroporation step on the cell product—and may also interface with common components, such as components of a fluidic bus line (e.g., pumps, valves, sensors, etc.). An advantage of such split module/instrument designs is that expensive components (e.g., motors, sensors, heaters, lasers, etc.) may be retained in the instruments of the system while multiple cartridges are processed. The use of disposable cartridges may eliminate the need, in such embodiments, to sterilize cartridges between use. Furthermore, the utilization of shared instruments (e.g. electroporation instrument, CCE instrument, MACS instrument, sterile liquid transfer instrument, FACS instrument, and the like) may be increased since a plurality of the instruments may be utilized simultaneously in parallel by a plurality of cell manufacturing processes. In contrast, conventional semi-automated instruments (e.g., Miltenyi Prodigy) have instrument components that sit idle and are incapable of simultaneous parallel use.

### Fluid Connector

Currently, there is no automated, multi-use sterile fluid connector solution for cell therapy production where a set of sterile fluid connectors are capable of multiple connection and disconnection cycles with a system. For example, conventional sterile fluid connectors are typically single-use devices and are thus expensive and labor intensive. Generally, the fluid connectors described herein include a plurality of sealed enclosures between a sterile portion (e.g., fluid connector lumen or cavity) and an external (e.g., non-sterile) ambient environment, thereby facilitating aseptic control of a fluid connector and devices coupled thereto. The fluid connectors described herein may be a durable component that may be reused for multiple cycles while maintaining sterility and/or bioburden control. For example, the fluid connector may be sterilized using a sterilant without harming the cell product or other biological material.

In some embodiments, a sterile manufacturing system as described herein may utilize one or more sterile fluid connectors and have a configuration suitable to be manipulated by a robot such as a robotic arm. The sterile fluid connectors described herein enable the transfer of fluids in an automated, sterile, and metered manner for automating cell therapy manufacturing. Automating cell therapy manufacturing may in turn provide lower per patient manufacturing costs, a lower risk of process failure, and the ability to meet commercial scale patient demand for cell therapies. In some embodiments, sterile fluid connectors may increase one or more of sterility, efficiency, and speed by removing a human operator from the manufacturing process. An automated and integrated sterilization process as described herein may be applied to the fluid connector to maintain sterility of the system. For example, the fluid connector may maintain sterility through multiple connection/disconnection cycles between separate sterile closed volume fluid devices (e.g., enclosure, container, vessel, cartridge, instrument, bioreactor, enclosed vessel, sealed chamber). Accordingly, the systems, devices, and methods described herein may reduce the complexity of a sterilization process, reduce energy usage, and increase sterilization efficiency.

In some embodiments, a fluid connector may comprise a first connector configured to mate with a second connector (e.g., male connector and female connector). Respective proximal ends of the connectors may be configured to connect (e.g., be in fluid communication, form a fluid pathway) with respective fluid devices in order to transfer one or more of fluid (e.g., liquid and/or gas) and biological material (e.g., cell product) between the fluid devices. The distal ends of the connectors may comprise ports configured to mate with each other. The fluid connector may also comprise a sterilant port configured to facilitate sterilization of a chamber within the distal ends of the first and second connectors. The fluid connector may be sterilized before or after connection as desired to ensure sterility. In this manner, the fluid connector may be reused for multiple connection and disconnection cycles.

In some embodiments, a cell processing system utilizing the fluid connectors described herein may comprise a robot configured to operate the fluid connector and a controller configured to control the robot to manipulate (e.g., move, connect, open, close, disconnect) the first and second connectors together (without human interaction) while maintaining sterility of the fluid connector and a plurality of fluid devices, thereby further reducing the risk of contamination. The fluid devices may be one or more of an instrument, cartridge, and the like.

### II. Cell Processing Methods

### Cell Selection System

The cell processing methods described herein may utilize a cell selection system (or cell separation system) configured to separate cells based on predetermined criteria. For example, cells may be separated based on physical characteristics such as size and/or density using, for example, a counterflow centrifugation elutriation instrument. Cells may also be separated based on the presence of predetermined antigens of a cell using, for example, a magnetic-activated cell selection instrument. In some embodiments, a cell selection system comprising modules for these separation methods may facilitate one or more cell processing steps including, but not limited to, cell concentration, cell dilution, cell washing, buffer replacement, and magnetic separation. The cell selection systems described herein may increase throughput and cell yields output, in a compact and portable structure. For example, prior to magnetically separating cells, a suspension of cells may be mixed with magnetic reagents in excess or at a predetermined concentration (e.g., cells/ml). Likewise, after magnetically separating cells, the cells may be washed in a solution (e.g., suitable buffered solution).

In some embodiments, a cell separation system may comprise a rotor configured for counterflow centrifugation elutriation of cells in a fluid, a first magnet configured to magnetically rotate the rotor and separate the cells from the fluid in the rotor, a flow cell in fluid communication with the rotor and configured to receive the cells from the rotor, and a second magnet configured to magnetically separate the cells in the flow cell.

In some embodiments, a CCE module may be integrated into a cartridge to enable a cell processing system to separate cells based on cell size and/or density. In some embodiments, a cell separation system may comprise a housing comprising a rotor configured to separate cells from a fluid (e.g., separate cells of different size and/or density from cells that remain in the fluid), and a magnet configured to magnetically rotate the rotor. The housing may be configured to move relative to the magnet or vice versa (e.g., move the magnet relative to the housing). The CCE modules described herein may provide cell separation within a compact and portable housing where the magnet may be disposed external to the housing (e.g., magnet disposed within a CCE instrument of the workcell).

In some embodiments, a compact rotor that may aid cartridge integration may comprise input and output fluid conduits extending from the rotor towards opposing sides of a rotor housing. For example, a rotor may comprise a first side comprising a first fluid conduit and a second side comprising a second fluid conduit where the second side is opposite the first side. An elutriation chamber (e.g., cone) may be coupled between the first fluid conduit and the second fluid conduit.

In some embodiments, a method of separating cells from a fluid may comprise moving a rotor towards a magnet, the rotor defining a rotational axis, flowing the fluid through the rotor, rotating the rotor (e.g., magnetically) about the rotational axis using the magnet while flowing the fluid through the rotor, and moving the rotor away from the magnet.

In some embodiments, a method of separating cells from a fluid may comprise flowing the fluid comprising the cells into a flow cell. A set of the cells may be labeled with magnetic particles. The set of cells may be magnetically attracted towards a magnet array for a dwell time, and the set of cells may flow out of the flow cell after the dwell time.

In some embodiments, a flow cell may comprise an elongate cavity having a cavity height and a magnet array comprising a plurality of magnets, each of the magnets spaced apart by a spacing distance. A predetermined ratio between the cavity height to the spacing distance may optimize magnetic separation of the cells in the flow cell.

### Electroporation

In some embodiments, an electroporation system as described herein may be configured to facilitate one or more of transduction and transfection of cells. As described in more detail herein, a volume of fluid (e.g., first batch) comprising cells may be physically separated from a subsequent volume of fluid (e.g., second batch, third batch) comprising cells by a gas (e.g., air gap). Applying an electroporation signal (e.g., voltage pulse, waveform) separately to each discrete batch of fluid may improve electroporation efficiency and thus increase throughput. In some embodiments, active electric field compensation may similarly improve electroporation efficiency and throughput.

In some embodiments, an electroporator module of a cartridge may comprise a fluid conduit configured to receive a first fluid comprising cells and a second fluid (e.g., gas, oil), a set of electrodes coupled to the fluid conduit, a pump coupled to the fluid conduit, and a controller comprising a processor and memory. The controller may be configured to generate a first signal to introduce the first fluid into the fluid conduit using the pump, generate a second signal to introduce the second fluid into the fluid conduit such that the second fluid separates the first fluid from a third fluid, and generate an electroporation signal, via an electroporation instrument of the workcell, to electroporate the cells in the fluid conduit using the set of electrodes.

In some embodiments, a method of electroporating cells may comprise receiving a first fluid comprising cells in a fluid conduit, receiving a second fluid comprising a gas in the fluid conduit to separate the first fluid from a third fluid, and applying an electroporation signal to the first fluid to electroporate the cells.

In some embodiments, a method of electroporating cells may comprise receiving a first fluid comprising cells in a fluid conduit, applying a resistance measurement signal to the first fluid using a set of electrodes, measuring a resistance between the first fluid and the set of electrodes, and applying an electroporation signal to the first fluid based on the measured resistance.

### Bioreactor

In some embodiments, a bioreactor module of the cartridge may comprise an enclosure comprising a base and a sidewall, and a gas-permeable membrane coupled to one or more of the base and the sidewall of the enclosure. The gas-permeable membrane may aid cell culture. In some embodiments, a bioreactor instrument of the cell processing system may be configured to provide signals to the bioreactor module of the cartridge to control an agitator coupled to the bioreactor module. The agitator may be configured to agitate the bioreactor in cooperation with the bioreactor instrument of the workcell and based on orbital motion.

### Cell Processing Workflows

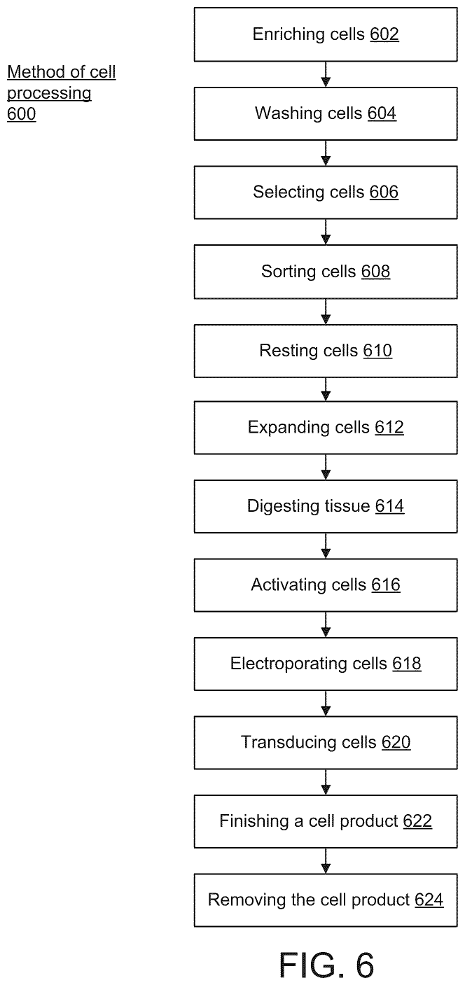

Generally, the systems and devices described herein may perform one or more cell processing steps to manufacture a cell product. FIG. 6 is a flowchart of a method of cell processing 600. The method 600 may include enriching a selected population of cells in a solution (e.g., fluid) 602. For example, the solution may be conveyed to a CCE module of a cartridge via a liquid transfer bus. A robot may be operated to move the cartridge to a CCE instrument so that the CCE module interfaces with the CCE instrument. The CCE instrument may be operated to cause the CCE module to enrich the selected population of cells. Additionally or alternatively, the cell product may be introduced into and out of the cartridge via a sterile liquid transfer port (either manually or automatically) for any of the steps described herein. In some embodiments, the cartridge may be sterilized in a feedthrough port (either manually or automatically).

In some embodiments, a selected population of cells in the solution may be washed 604. For example, the solution may be conveyed to the CCE module of the cartridge via the liquid transfer bus. A robot may be operated to move the cartridge to the CCE instrument so that the CCE module interfaces with the CCE instrument. The CCE instrument may be operated to cause the CCE module to remove media from the solution, introduce media into the solution, and/or replace media in the solution.

In some embodiments, a population of cells in the solution may be selected 606. For example, the solution may be conveyed to a selection module of the cartridge via the liquid transfer bus. The robot may be operated to move the cartridge to a selection instrument so that the selection module interfaces with the selection instrument. The selection instrument may be operated to cause the selection module to select the selected population of cells.

In some embodiments, a population of cells in the solution may be sorted 608. For example, the solution may be conveyed to a sorting module of the cartridge via the liquid transfer bus. The robot may be operated to move the cartridge to a sorting instrument so that the sorting module interfaces with the sorting instrument. The sorting instrument may be operated to cause the sorting module to sort the population of cells.

In some embodiments, the solution may be conveyed to a bioreactor module of the cartridge via the liquid transfer bus to rest 610. For example, the robot may be operated to move the cartridge to a bioreactor instrument so that a bioreactor module interfaces with the bioreactor instrument. The bioreactor instrument may be operated to cause the bioreactor module to maintain the cells at a set of predetermined conditions.

In some embodiments, the cells may be expanded in the solution 612. For example, the solution may be conveyed to the bioreactor module of the cartridge via the liquid transfer bus. The robot may be operated to move the cartridge to the bioreactor instrument so that the bioreactor module interfaces with the bioreactor instrument. The bioreactor instrument may be operated to cause the bioreactor module to expand the cells by cellular replication.

In some embodiments, tissue may be digested by conveying an enzyme reagent via the liquid transfer bus to a module containing a solution containing a tissue such that the tissue releases a select cell population into the solution 614.

In some embodiments, a selected population of cells in the solution may be activated by conveying an activating reagent via the liquid transfer bus to a module containing the solution containing the cell product 616.

In some embodiments, the solution may be conveyed to an electroporation module of the cartridge via the liquid transfer bus and receive an electroporation signal to electroporate the cells in the solution 618. For example, the robot may be operated to move the cartridge to an electroporation instrument so that the electroporation module interfaces with the electroporation instrument. The electroporation instrument may be operated to cause the electroporation module to electroporate the selected population of cells in the presence of genetic material.

In some embodiments, an effective amount of a vector may be conveyed via the liquid transfer bus to a module containing the solution containing the cell product, thereby transducing a selected population of cells in the solution 620.

In some embodiments, a formulation solution may be conveyed via the liquid transfer bus to a module containing the cell product to generate a finished cell product 622. For example, the finished cell product may be conveyed to one or more product collection bags. In some embodiments, finishing a cell product may comprise one or more steps of washing cells, concentrating cells, exchanging a buffer of the cells with a formulation buffer, and dosing cells in the formulation buffer in predetermined quantities into one or more product collection bags and/or vessels.

In some embodiments, the cell product may be removed, either manually or automatically, from the cartridge to harvest the cells 624.

In some embodiments, the cell product may comprise one or more of an immune cell genetically engineered chimeric antigen receptor T cell, a genetically engineered T cell receptor (TCR) cell, a hematopoietic stem cell (HSC), and a tumor infiltrating lymphocyte (TIL). In some embodiments, the immune cell may comprise a natural-killer (NK) cell.

Methods of cell processing may include a subset of cell processing steps in any suitable order. For example, the method of cell processing may include, in order, the enrichment step 602, the selection step 606, the activation step 616, the transduction step 620, the expansion step 612, and the harvesting step 624. In some embodiments, the method of cell processing may include, in order, the enrichment step 602, the selection step 606, the resting step 610, the transduction step 620, and the harvesting step 624. In some embodiments, the method of cell processing may include, in order, the tissue-digestion step 620, the washing step 604, the activation step 616, the expansion step 612, and the harvesting step 624.

Generally, the methods described herein may offload the complex steps performed in cell processing operation to a set of instruments, thereby reducing the cost of the cartridge. In some embodiments, the cartridge may contain the cell product (e.g., solution containing cells) throughout a manufacturing process, with different instruments interfacing with the cartridge at appropriate times to perform one or more cell processing steps. For example, a cell processing step may comprise conveying cells and reagents to each of the modules within the cartridge. A set of instruments interfacing with a corresponding set of modules within the cartridge facilitates process flexibility where a workcell may be customized with a predetermined set of instruments for a predetermined cell therapy product. For example, the order of cell processing steps may be customized for each cell product as described in more detail herein.

In some embodiments, a cell product may be retained within the cartridge throughout a manufacturing process (e.g., workflow). Additionally, or alternatively, the cell product may be removed from the cartridge for one or more cell processing steps, either manually by an operator, or automatically through a fluid connector (e.g., SLTP) or other access ports on the cartridge. The cell product may then be returned to the same cartridge, transferred to another cartridge, or split among several cartridges. In some embodiments, one or more cell processing steps may be performed outside the cartridge. In some embodiments, processing within the workcell may facilitate sterile cell processing within the cartridge.

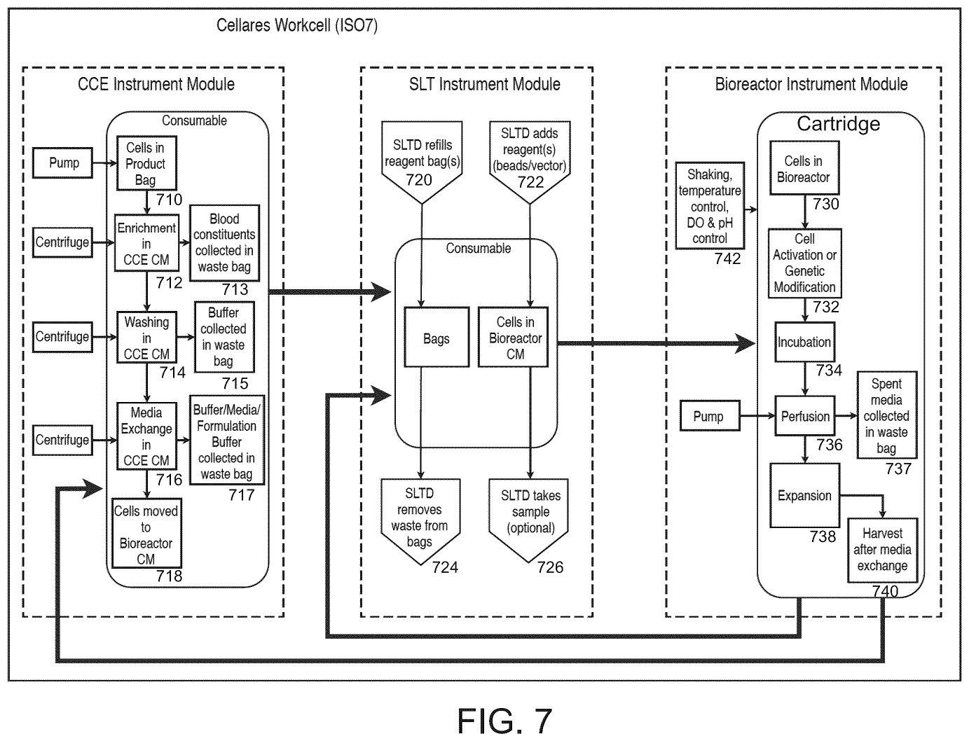

FIG. 7 is a flowchart of a method of cell processing and illustrates cell processing steps performed on a cartridge within a workcell including a CCE instrument module, a sterile liquid transfer (SLT) instrument module, and a bioreactor instrument module. The cartridge may be configured to interface with any of the CCE instrument module, SLT instrument module, and bioreactor instrument module to perform one or more cell processing steps. For example, a robot (or operator) may be configured to move a cartridge between any of the modules of the workcell. A pump head in an instrument may engage the cartridge in order to convey fluids between the modules of the cartridge, into or out of various reservoirs in the cartridge, and/or through ports that permit reagents to be added or removed from the cartridge.

In some embodiments, the CCE instrument module may comprise a pump and centrifuge configured to interface with a cartridge. The SLT instrument module may comprise one or more fluid connectors be configured to interface with one or more of a bag and bioreactor of a cartridge. The bioreactor instrument module may comprise one or more sensors, temperature regulators, pumps, agitators, and the like, and be configured to interface with the cartridge. In some embodiments, the cell product may be contained within the cartridge throughout cell processing.

A method of cell processing depicted in FIG. 7 may include moving a fluid (e.g., cells in solution) in a product bag to a CCE module (e.g., rotor) of a cartridge using a pump 710. In some embodiments, the fluid may be enriched using the CCE module 712. For example, blood constituents may be collected in a waste bag 713. In some embodiments, the fluid may be washed using the CCE module 714. For example, buffer may be collected in a waste bag 715. In some embodiments, media may be exchanged using the CCE module 716. For example, one or more of buffer (e.g., formulation buffer) and media may be collected in a waste bag 717. In some embodiments, fluid may be moved to a bioreactor of the cartridge 718.

In some embodiments, a fluid connector may fill a bag with a reagent 720. In some embodiments, a reagent (e.g., bead, vector) may be added to a bioreactor of a cartridge 722. In some embodiments, a fluid connector removes waste from a bag 724. In some embodiments, a fluid connector may optionally remove a sample from a bioreactor.

In some embodiments, cells may be moved to a bioreactor 730. In some embodiments, the cells may undergo activation or genetic modification 732. In some embodiments, the cells may undergo incubation 734. In some embodiments, the cells may undergo perfusion using a pump 736. For example, spent media may be collected in a waste bag 737. In some embodiments, cells may undergo expansion 738. In some embodiments, cells may be harvested after media exchange 740.

In some embodiments, loading and removing of cell product into and out of the cartridge may be performed in the system or outside the system. In some embodiments, the cartridge is loaded bedside to the patient or donor and then delivered to a cell processing system in or near the hospital or shipped to a facility where the cell processing system is installed. Likewise, the cell product may be removed from the cartridge after processing either at a facility or closer to the intended recipient of the cell product (the patient). Optionally, the cell product is frozen before, during, or after the methods of the disclosure-optionally after addition of one or more cryoprotectants to the cell product. In some embodiments, the system comprises a freezer and/or a liquid nitrogen source. In some embodiments, the system comprises a water bath or a warming chamber containing gas of controlled temperature to permit controlled thawing of the cell product, e.g. a water bath set to between about 20° C. and about 40° C. In some embodiments, the cartridge is made of materials that resistant mechanical damage when frozen.

Described below are methods of transforming user-defined cell processing operations into cell processing steps using the automated cell processing systems and devices described herein. In some embodiments, cell processing operations are received and transformed into cell processing steps to be performed by the system given a set of predetermined constraints. For example, a user may input a set of biologic process steps and corresponding biologic process parameters to be executed by a cell processing system. Optionally, process parameters may be customized for each cartridge or sets of cartridges.

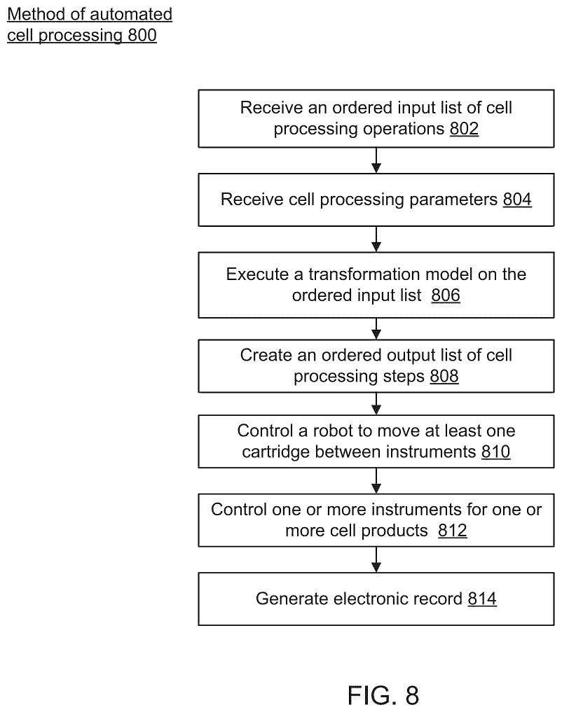

FIG. 8 is a flowchart that generally describes an embodiment of a method of automated cell processing. The method 800 may include receiving an ordered input list of cell processing operations 802. For example, a set of more than one ordered input list of cell processing operations may be received to be performed on more than one cartridge on an automated cell processing system. For example, as shown in the GUI 3100 of FIG. 31 and described in more detail herein, one or more biologic process inputs (e.g., available operations) such as enrichment, MACS selection, activation, transduction, transfection, expansion, and inline analysis may be selected as an ordered input list of cell processing operations. Furthermore, GUI 3400 of FIG. 34 illustrates a complete ordered input list of cell processing operations (e.g., set of selected operations) 3420 selected by a user.

In some embodiments, one or more sets of cell processing parameters may be received 804. Each set of cell processing parameters may be associated with one of the cell processing operations. Each set of cell processing parameters may specify characteristics of the cell processing step to be performed by the instrument at that cell processing step. For example, the GUI 2200 of FIG. 22 illustrates reagent and container parameters, the GUI 2900 of FIG. 29 illustrates an example of a process parameter, the GUI 2990 of FIG. 26 illustrates an example of a preprocess analytic, and the GUI 3200 of FIG. 32 illustrates an example of a set of activation settings.

In some embodiments, a transformation model may be executed on the ordered input list 806. In some embodiments, the transformation model may comprise constraints on the ordered output list determined by a predetermined configuration of the automated cell processing system. For example, the constraints may comprise information on the configuration of the automated cell processing system.

In some embodiments, the constraints may comprise one or more of a type and/or number and/or state of instruments, a type and/or number and/or state of modules on the cartridge, a type and/or number of reservoirs on the cartridge, a type and/or number of sterile liquid transfer ports on the cartridge, and number and position of fluid paths between the modules, reservoirs, and sterile liquid transfer ports on the cartridge.

In some embodiments, a set of predetermined constraints may be placed on a set of the process control parameters. For example, the volume and/or the type of reagents used may be constrained based on the size of the system and/or products manufactured. Other process parameter constraints may include, but is not limited to, one or more or temperature, volume, time, pH, cell size, cell number, cell density, cell viability, dissolved oxygen, glucose levels, volumes of onboard reagent storage and waste, combinations thereof, and the like. For example, the GUI 2200 of FIG. 22 depicts that a reagent has a volume per unit of 30 ml and a required volume of 54 ml, and a container has a volume per unit of 75 ml. The GUI 3200 of FIG. 32 depicts that an activation concentration is 12 mg/L, an activation culture time is 1100 seconds, activation temperature is 18° C., and a gas mix includes 21% oxygen, 78.06% nitrogen, and 0.04% of carbon dioxide. These constraints may be applied by a transformation model to generate an ordered output list of cell processing steps that affect how one or more of the robot, instrument, and cartridge are operated and the cell product manufactured.

In some embodiments, the order of operations may be constrained based on hardware constraints. For example, the robot may be limited to moving one cartridge at a time. Similarly, an instrument may be constrained to operating on a predetermined number of cartridges at once.

In some embodiments, as illustrated in the GUI 3100 of FIG. 31, a load product operation must be the first operation performed and may be performed once for each process. A fill and finish operation may always be the last operation performed before product completion and may be performed once for each process.

In some embodiments, the system may prevent the user from executing a set of operations in an order that cannot be performed by the system.

In some embodiments, a notification (e.g., warning, alert) may be output if a user orders a set of operations in a “non-standard” manner. For example, a notification may be output if the same type of operation is repeated sequentially (e.g., enrichment immediately followed by enrichment). Similarly, a notification may be output if an operation (e.g., selection, activation) is used two times or more within a given process when such an operation is typically used just once in a given process.

In some embodiments, an output of the transformation model may correspond to an ordered output list of cell processing steps capable of being performed by the system 808. For example, the transformation model may be executed on the sets of ordered input lists to create the ordered output list of cell processing steps. The output list of cell processing steps may control a robot, cartridge, and one or more instruments.

In some embodiments, the ordered output list is performed by the system to control a robot to move one or more cartridges each containing a cell product between the instruments 810. For example, the MACS selection process selected by the user may correspond to the robot 230 of FIG. 2 moving the cartridge 250 to the cell selection instrument 216 from, for example, another instrument. In some embodiments, the ordered output list may comprise instructions for a robot to load a cartridge into the cell processing system. Furthermore, the robot may be configured to move the cartridge to a first instrument position.

In some embodiments, the ordered output list is further performed by the system to control one or more of the instruments to perform one or more cell processing steps on one or more cell products 812 of a respective cartridge. For example, the compute server rack 210 (e.g., controller 120) may be configured to control an electroporation module 220 configured to apply a pulsed electric field to a cell suspension of a cartridge 250. In some embodiments, the ordered output list may comprise instructions for an instrument (e.g., bioreactor) to process the product (e.g., transfer the cell product from a small bioreactor module to a large bioreactor module). Furthermore, the instrument may be further configured to operate under a set of process parameters (e.g., 9 hour duration, pH of 6.7, temperature between 37.3° C. and 37.8° C., mixing mode 3). As another example, the ordered output list may comprise instructions to operate a sterile liquid transfer module to perform one or more of removing waste from a cartridge, adding media to the cartridge, and adding a MACS reagent to the cartridge.

In some embodiments, one or more electronic batch records may be generated 814 based on the process parameters and data collected from sensors during process execution. Batch records generated by the system may include process parameters, time logging, sensor measurements from the instruments, QC parameters determined by QC instrumentation, and other records.

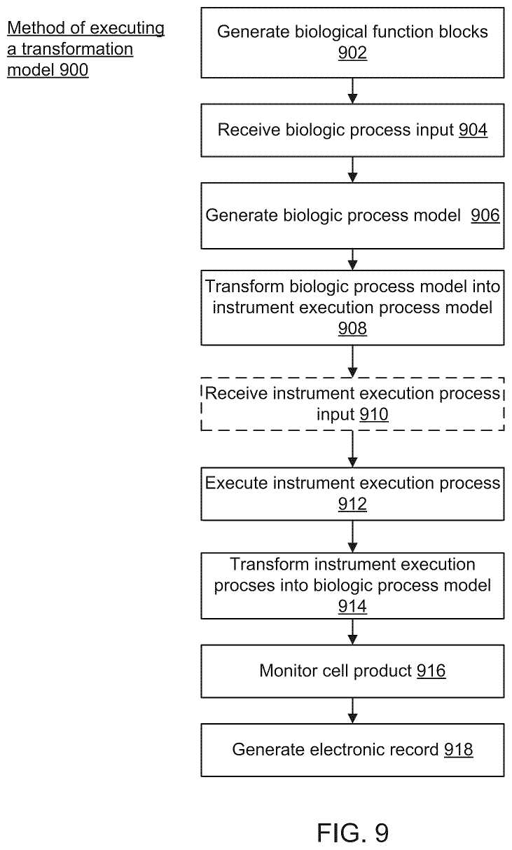

FIG. 9 is a flowchart that generally describes an embodiment of a method of executing a transformation model 900. In some embodiments, one or more biological functions may be generated and output to a user. For example, a set of configurable biological function blocks may be displayed on a graphical user interface for user selection. The GUI may enable a user to select and order the biological function blocks and define biological control parameters. One or more control parameters of the biologic function blocks may be modified by a user if desired. In some embodiments, one or more biologic function templates may be generated comprising a predefined sequence of biological function blocks. One or more biological control parameters of the biologic function templates may be modified by a user if desired.

In some embodiments, a cell processing system may be configured to receive and/or store one or more biologic function (e.g., process) inputs from the user 904. For example, a user may select one or more predefined biological function templates.

In some embodiments, a biologic process model (e.g., process definition) may be generated based on the biologic process inputs 906. In some embodiments, a biologic process model may include one or more of enrichment, isolation, MACS selection, FACS selection, activation, genetic modification, gene transfer, transduction, transfection, expansion, formulation (e.g., harvest, pool), cryopreservation, T cell depletion, rest, tissue digestion, washing, irradiation, co-culture, combinations thereof, and the like.

In some embodiments, the biologic process model may be transformed into an instrument execution process model 908. For example, each biological function block in the biological process model may correspond to an ordered list of cell processing system operations with corresponding hardware control parameters. The instrument execution process model may comprise the sequence of hardware operations corresponding to the biologic process model. As described herein, the transformation model may comprise one or more constraints.

Optionally, in some embodiments, a cell processing system may be configured to receive and/or store one or more instrument execution process inputs from the user 910. For example, a user may modify the transformed instrument execution process model if desired. The user may select specific hardware components to perform certain steps, modify timing parameters, and the like.

In some embodiments, the instrument execution process may be executed to generate the cell product 912. For example, the cell processing system at run-time may process the cell product through the system as defined by the instrument execution process model.

In some embodiments, an instrument execution process may be executed 912. In some embodiments, an instrument execution process model may be transformed back into a biologic process model 914. This progress of the biologic process model may be output (e.g., displayed) to a user for monitoring. For example, the instrument execution process model may comprise one or more references (e.g., pointers) back to the biological process model so that run-time execution progress may be reported against the biological process model.

In some embodiments, a cell product may be monitored 916. For example, the GUIs 3500 and 3600 of respective FIGS. 35 and 36 illustrate sensor data monitored by the system for a plurality of products. For example, a number of viable cells and a status of a process (e.g., as a function of percentage completion) may be graphically illustrated for a user.

In some embodiments, an electronic record may be generated based on the monitored data 918. For example, one or more electronic batch records may be generated in compliance with, for example, 21 CFR regulations.

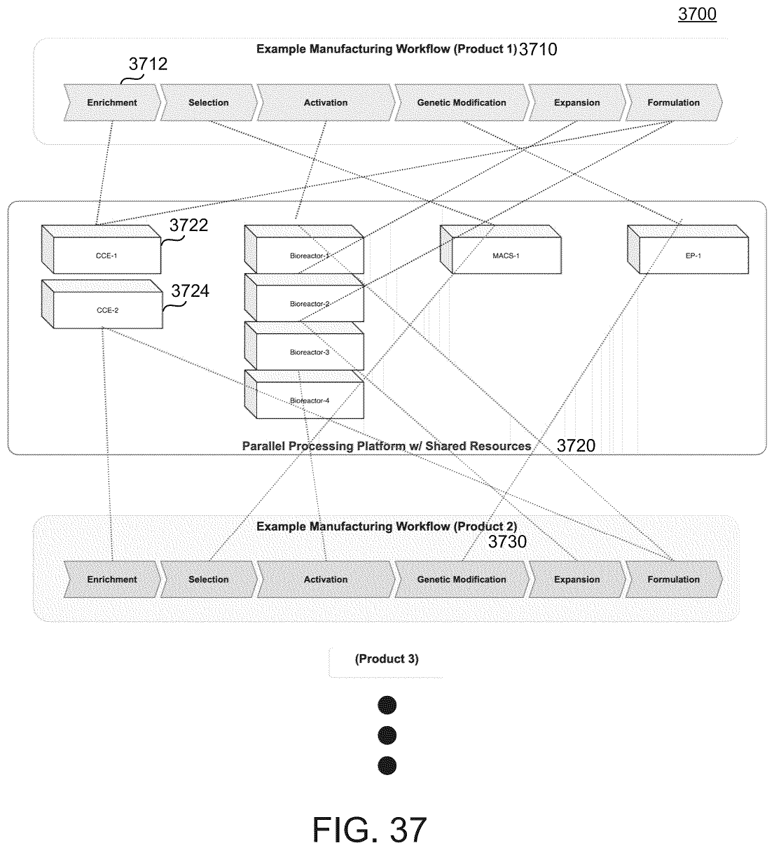

FIG. 37 is a block diagram of an illustrative embodiment of a manufacturing workflow 3700 comprising a processing platform 3720 (e.g., system 100, workcell 110, 200, 201) configured to generate a plurality of cell products (e.g., first product, second product, third product) in parallel. For example, a first workflow 3710 for a first product may include a plurality of biologic processes 3712 executed in a predetermined sequence using corresponding elements 3722 (e.g., hardware) of the platform 3720. Simultaneously, a second workflow 3730 for a second product may execute a predetermined sequence of biologic processes 3730 using corresponding elements 3724 of the platform 3720. In this manner, hardware resources of the platform 3720 may be efficiently utilized to increase throughput. In some embodiments, about 2, 3, 4, 5, 6, 7, 8, 9, 10, or more cell products may be manufactured simultaneously on the platform 3720.

### Planning

To this end, and referring now to FIGS. 10-18, planning parallel workflows presents challenges not previously encountered. The simulator of the present disclosure enables modeling and analysis of system performance under various scenarios including cartridge introduction rate and process workflow. Outputs of the simulator may include throughput, instrument contention resulting from parallel processing, and/or cartridge utilization and timing. This may include indications to a user of an optimal time to introduce a new cartridge. In some embodiments, the simulator, or simulation model, can illustrate the interactions between a user and the workcell, which consists of a robotic arm, stations, and various instruments. The simulator can be based on various inputs, shown in FIG. 10, including interactions between the user, a cartridge (CC), a SLTD, a material handling system (MHS), and a workcell comprising a robot and, as instruments, a bioreactor system (BRS), a transcription factor system (TFS), a biosterilization cabinet (BSC), a cell separation system (CSS), a sterile liquid transfer instrument (SLT), and a reagent vault system (RVS) are shown as lines entering and exiting each “node”. The below Drawings will be described in the context of at least one cartridge, which may be a cartridge, a SLTD, and the like.

Methods for planning parallel biomanufacturing processes, which can be implemented within the system of FIG. 10, will now be described with reference to FIGS. 11A-11G and from the perspective of an input device configured to receive input data from a user. The input device may be a touch screen, a keyboard, a mouse, and the like and may be operatively coupled to a display. A user input to the input device (e.g., keyboard, buttons) may be received and processed by a processor and memory of the system.

FIG. 11A is a high-level flow diagram of a method 1100 of the present disclosure. At step 1105 of method 1100, one or more inputs associated with the parallel biomanufacturing processes to be performed on cell samples within each of a plurality of cartridges may be received. In some embodiments, the one or more inputs may include a desired number of cartridges to be introduced to the workcell and/or an introduction rate of cartridges to the workcell, types of instruments within the workcell, a number of each type of instrument within the workcell, different states of the instruments within the workcell (e.g., vacant, occupied), one or more process steps associated with each instrument, each cartridge, and/or each process workflow, and allowable variation within each of the one or more process steps associated with each instrument, each cartridge, and/or each process workflow. For instance, the allowable variation can include timing margins for each of the one or more process steps. The timing margins can indicate which of the one or more process steps are exchangeable, distributable, and the like. As will be described later, the simulation can utilize these timing margins when reaching an optimal solution. Additionally, or to this end, the one or more inputs may include requirements and constraints based on a desired outcome of the biomanufacturing processes. For example, a desired outcome (e.g., a target result) may include a desired cell product selected from any of the following: hematopoietic stem cells (HSCs), chimeric antigen receptor (CAR) T cells, NK cells, tumor infiltrating lymphocytes (TILs), T-cell receptors (TCRs), regulatory T cells (T regs), gamma delta (γδ) T-cell. It can be appreciated that each of these cellular products have their own unique manufacturing processes, and accordingly, dictate their own unique constraints and requirements.

In an example, the one or more inputs may require that one new cartridge be introduced to the workcell every 24 hours, that the biomanufacturing process require 360 hours (˜15 days) with a total of 67 steps (not including robot pickup and drop offs, which take approximately 3 minutes), that the biomanufacturing process requires six components, including a bioreactor instrument, a sterile liquid transfer instrument, a centrifugal elutriation instrument, a magnetic separator, a robotic arm, and a feedthrough, and the desired workflow of the biomanufacturing process to be executed, such as e.g., a long CAR-T process, a medium CAR-T process, or a short CAR-T process, is a long CAR-T process.

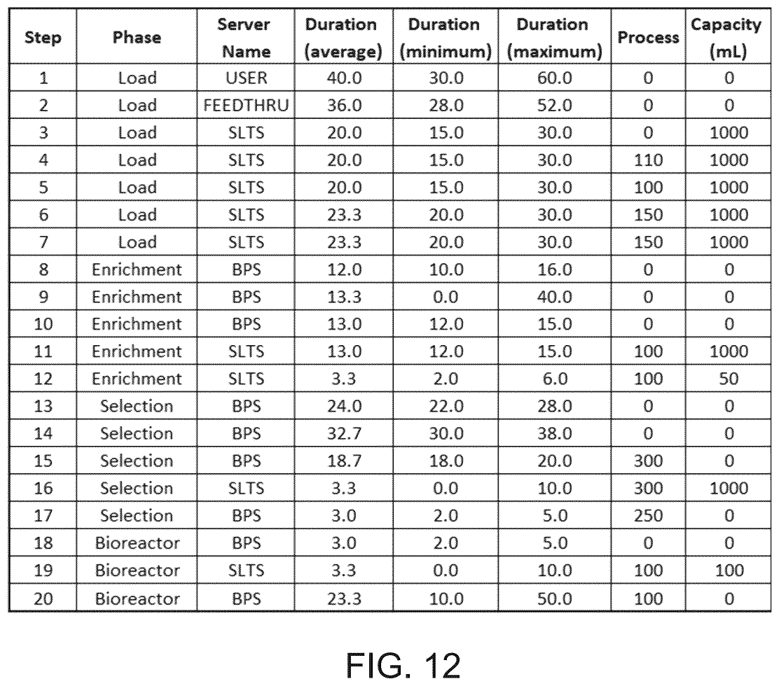

FIG. 12 depicts an exemplary list of one or more possible inputs and associated biomanufacturing process steps. As shown, the one or more inputs for the biomanufacturing process workflow may include a step, a phase (e.g., a process which may comprise steps), a duration, a process value (e.g., each of the numbers “110”, “100”, “150” etc. can represent a different liquid/reagent), and a capacity value. A phase may be, for instance, “cell expansion”, which may comprise the steps of “cell culturing”, “cell sampling”, “reagent addition”, “waste removal”, and the like. In embodiments, the phase may include movement of a cartridge by a robotic arm to or from locations associated with an instrument, moving, by the robotic arm, one or more closed volume fluid devices (e.g., a sterile liquid transfer device) between a fluid storage vault (e.g., reagent vault system) and the sterile liquid transfer instrument, transferring waste generated by the plurality of cartridges to a corresponding closed volume fluid device within the fluid storage vault, and transferring cell therapy samples generated by the plurality of cartridges to a corresponding closed volume fluid device within the fluid storage vault. The information in the chart can be used at sub process 1110 of method 1100 to perform a simulation with various outputs, as disclosed below.

At sub process 1110 of method 1100, a simulation may be performed based on the one or more inputs received at step 1105. In some embodiments, the simulation may be an analog simulation or a linear simulation. In some embodiments, and as described above, the simulation may be a finite-state machine model, a Markov chain model, a stochastic process model, a queuing theory model, a transaction-level model, a job-shop model, and a discrete-event simulator, which models the parallel processes within the workcell as a sequence of events in time. Discrete-event simulation is a method used to model real world systems that can be decomposed into a set of logically separate processes that autonomously progress through time. Each event occurs at a particular instant and marks a change of state in the system. Between consecutive events, no change in the system is assumed to occur, thus the simulation can directly jump to the occurrence time of the next event.

The simulation of sub process 1110 of method 1100 “executes” an entire sequence of cartridges through a “virtual” instrument arrangement and generates a timing diagram for each cartridge throughout a respective biomanufacturing process. The simulation includes queuing of cartridges as a function of instrument availability (i.e., contentions). This queuing is apparent in the outputs of the method 1100, which are described below.

Based on the simulation, or in some embodiments as part of the simulation (denoted by dashed lines to step 1120), different types of outputs can be generated at step 1120 of method 1100, thereby enabling observation of the system behavior and the optimization of resource allocation. In embodiments, the different types of outputs may include periodic Gantt charts, biomanufacturing workcell component performance monitoring, and the potential contentions.

In some embodiments, an output based on the simulation comprises a periodic Gantt chart of workflow processes versus time. Through post-processing of the periodic Gantt chart, insights into the simulated runs, such as internal sterile liquid transfer device inventory vs time, contention per instrument, and the like can be cleaned. Moreover, these periodic Gantt plots in turn can be analyzed to optimize system configurations and/or cartridge introduction schedules to minimize contention.

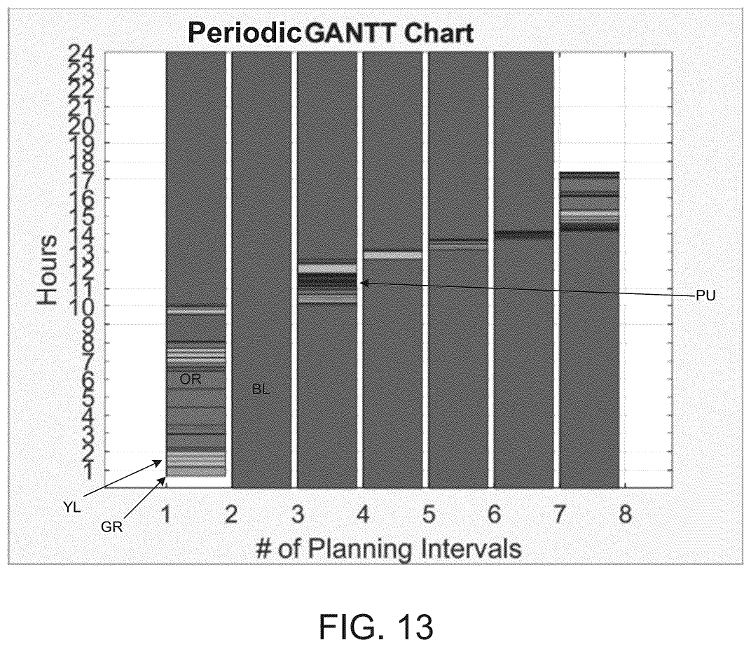

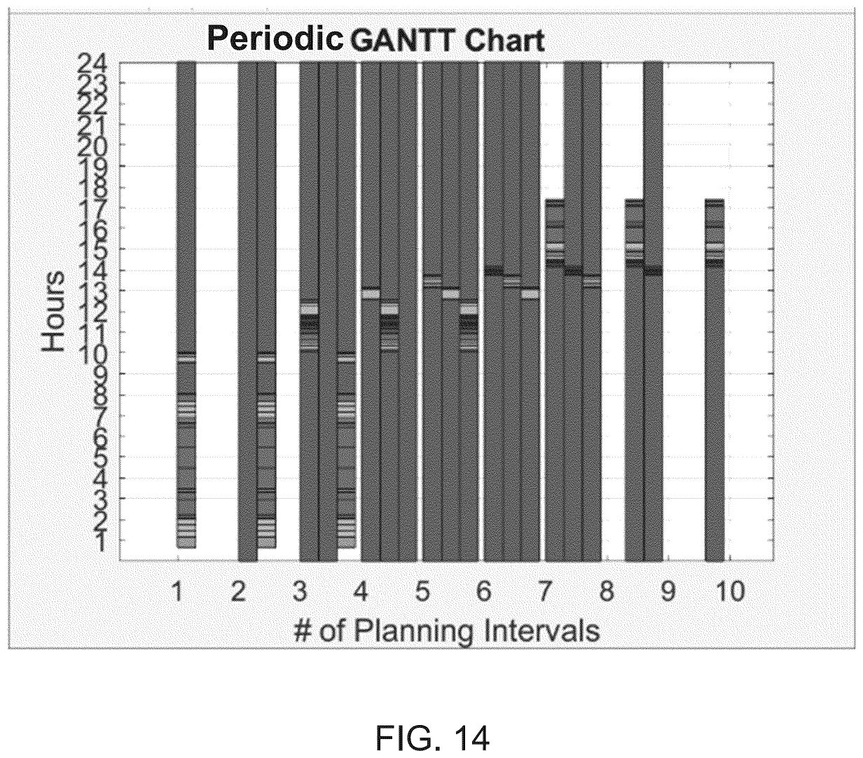

An exemplary periodic Gantt chart is shown in FIG. 13. In FIG. 13, utilization of a single cartridge is shown during 24-hour periods (number of planning intervals, or periods, on the x-axis, while the number of hours in each planning interval is on the y-axis). An additional exemplary periodic Gantt chart is shown in FIG. 14. Similar to FIG. 13, FIG. 14 displays number of planning intervals, or periods, on the x-axis, and the number of hours in each planning interval on the y-axis. However, unlike FIG. 13, the periodic Gantt chart of FIG. 14 features the introduction of a second cartridge during planning interval two and a third cartridge during planning interval three. As will be described later, it can be appreciated, from a visual perspective, the overlap of certain hues of adjacent bars within a particular planning period can indicate a contention.

Utilizing the periodic Gantt approach enables the system to derive a deterministic outcome with high customer usability from a process which includes variability. In other words, if the process continues to change and/or changes randomly, it is impossible to know for certain that the process will succeed for future-added cartridges. But, if the process can be reduced to a periodic function in which the impact of process deviations is limited, as is done herein, a deterministic outcome from a process which considers variability can be derived. To this end, the periodic Gantt approach reduces the biomanufacturing processes to periodic functions and provides a method of normalizing one or more workflows and scheduling to create a deterministic process output from variable inputs. Moreover, the periodic Gantt approach provides the ability to schedule multiple events on multiple days, enabling high customer usability via optimization.

In some embodiments, the Planning aspect of Periodic Gantt Planning and Plotting may implement conditions that are useful for a holistic approach. These conditions may include one or more of predefining a desired workflow period for usability, grouping steps in which high processing activities are occurring, defining phases in which process timing is long, and optimizing timing for process variability. In an embodiment, predefining a desired workflow period comprises defining a period to be 24 hours and stipulating that the workflow is to be optimized to operate within no-minimal contention in this period. In an embodiment, grouping steps in which high processing activities are occurring comprises separating the grouped steps by periods of long processing and/or long duration.

The process of grouping of steps, which can be useful for both simulation and visualization, may allow a simpler evaluation of process worst case conditions. If, for example, a 100-step is grouped into 5-sections of which only 3-sections have critical timing contention potential, then the equation for number of simulations drops from 1003 to 33 per cartridge. In an embodiment, defining phases in which process timing is long comprises estimating processing timing relative to process variability. For instance, appreciating that a long activation step may be ˜48 hours and multiple expansion phase steps may be between 24 hours and 48 hours, understanding that these steps can generally vary by greater than 10% can be used to compensate for undesired variation in the grouped steps. As an example, if a set of initial processing steps runs 3 hours long, the activation step may be reduced from 48 hours to 45 hours such that the timing on exiting activation has zero variability as a function of the input process timing.

In an embodiment, optimizing timing for process variability comprises distributing the timing margin between process groups to ensure that the impact of variability within the grouped processes is constrained to the group itself. Therefore, the allowable variation in the long processes can be utilized to normalize the process timing such that subsequent steps are completely independent of prior variation.

As shown in FIG. 11C, sub process 1110 of method 1100 incorporates at least a portion of these conditions when performing a simulation. For instance, at step 1111 of sub process 1110, each process of a biomanufacturing workflow is evaluated to determine whether they can form a group. Grouping processes, which may be an aspect of a block simulation and/or Gantt plotting, facilitates contention modeling by simplifying the problem to be solved in order to make high level contention risk decisions. Processes are grouped when they have relatively lower tolerance for variation and should, therefore, be run with minimal inter-step contention. Application of this blocked approach for a single cartridge is shown in FIG. 11D for exemplary purposes. The left image of FIG. 11D depicts, in a first planning interval, a number of vertically arranged rectangles, each different hue thereof being a separate process within the biomanufacturing workflow for the cartridge. The right image of FIG. 11D depicts, for the first planning interval, the same first, lower most block corresponding to introduction of the cartridge, a single rectangular block corresponding to a majority of the separate processes shown in the left image, whereby group variation is derived as a composite of variation of each step, and a same darkest hue block corresponding to time within the bioreactor. In this way, variation tolerance is built into the model. Non-bioreactor steps are treated as continuous, meaning that the cartridge “owns” the corresponding instrument for the duration of the group of steps, and are separated by long bioreactor steps, which are variation tolerant, allow for float in the start time of a group of steps. In other words, because the amount of time the cartridge spends in the bioreactor can often be varied, variation sensitive group steps can proceed when ready and compensated for by reducing or extending time within the bioreactor.

At step 1112 of sub process 1110 and at step 1113 of sub process 1110, respectively, the grouped processes exemplified in FIG. 11D can be scaled to maximum duration and their start times can be locked in position, as exemplified in FIG. 11E. In other words, to accommodate possible variation within each of the grouped processes, it can be assumed that this group (or block) of processes may require the maximum amount of time possible, thereby ensuring the processes will finish within the allotted time. Compensation can occur by reducing bioreactor times so that a next group start time is not impacted. The vertical lines within the first planning interval and the third planning interval of the left image of FIG. 11E indicate the time by which each group is to be extended. The right image of FIG. 11E illustrates the grouped processes after having been extended to the maximum possible time.

At step 1114 of sub process 1110, currently active cartridges can be modeled. An exemplary output of a model output (which may also be an output generated at step 1120 of method 1100) is shown in FIG. 11F. In FIG. 11F, utilization over time of the sterile liquid transfer system, which can be a rate limiting instrument, is shown. Arrows indicate moments of 100% utilization of the sterile liquid transfer system. In the example shown in FIG. 11F, a cartridge starting January 10^{th} is scheduled to complete on January 24^{th}, and no contention is present during that time. However, considering there is also a cartridge scheduled to start on December 31 and complete on January 13^{th}, there is a period on January 5^{th} when contention will possibly need to be dealt with.

In some embodiments, where new cartridges are being added to a workcell comprising currently active cartridges, a solution for cartridge introduction assumes processes associated with in-process cartridges are fixed. The new cartridges, therefore, can vary nominal start time by +/−2 hours. As before, all grouped processes with variation must fit without contention. Therefore, during implementation, a first cartridge is fit, each subsequent cartridge is fit within +/−2 hours, and failure to fit results in abortion.

In some embodiments, where new cartridges are being added to a workcell comprising currently active cartridges, a solution for cartridge introduction assumes all cartridge workflows/processes can be adjusted. Though challenging to account for, considering this variation would be the best step toward achieving an ideal fit.

In some embodiments, and with reference now to FIG. 11B, method 1100′ may be similar to method 1100 of FIG. 11A but may include a step of identifying potential contentions based on the simulation. The potential contentions may include at least one contention of a phase contention, an instrument contention, and a workflow step contention.

As before, method 1100′ of FIG. 11B proceeds first at step 1105′ of method 1100′, whereby one or more inputs associated with the parallel biomanufacturing processes to be performed on cell samples within each of a plurality of cartridges may be received. In embodiments, the one or more inputs may include an introduction rate of cartridges to the workcell, types of instruments within the workcell, a number of each type of instrument within the workcell, different states of the instruments within the workcell (e.g., vacant, occupied), one or more process steps associated with each instrument, each cartridge, and/or each process workflow, and allowable variation within each of the one or more process steps associated with each instrument, each cartridge, and/or each process workflow. For instance, the allowable variation can include timing margins for each of the one or more process steps. The timing margins can indicate which of the one or more process steps are exchangeable, distributable, and the like. As will be described later, the simulation can utilize these timing margins when reaching an optimal solution. Additionally, or to this end, the one or more inputs may include requirements and constraints based on a desired outcome of the biomanufacturing processes. For example, a desired outcome (e.g., a target result) may include a desired cell product selected from any of the following: hematopoietic stem cells (HSCs), chimeric antigen receptor (CAR) T cells, NK cells, tumor infiltrating lymphocytes (TILs), T-cell receptors (TCRs), regulatory T cells (T regs), gamma delta (γδ) T-cell. It can be appreciated that each of these cellular products have their own unique manufacturing processes, and accordingly, dictate their own unique constraints and requirements.

At sub process 1110′ of method 1100′, a simulation may be performed based on the one or more inputs received at step 1105′. In some embodiments, the simulation may be an analog simulation or a linear simulation. In some embodiments, and as described above, the simulation may be a finite-state machine model, a Markov chain model, a stochastic process model, a queuing theory model, a transaction-level model, and a discrete-event simulator, which models the parallel processes within the workcell as a sequence of events in time.

Based on the simulation, or in some embodiments, as part of the simulation (denoted by dashed lines to step 1120′), different types of outputs can be generated at step 1120′ of method 1100′, thereby enabling observation of the system behavior and the optimization of resource allocation. Concurrently, based on the simulation or, in some embodiments, as part of the simulation, potential contentions can be identified at step 1115′ of method 1100′. The potential contentions may include at least one contention of a phase contention, an instrument contention, and a workflow step contention. The potential contentions may be identified by visual evaluation of the output generated based on the simulation (or as part of the simulation) and/or identified by the processor of the workcell. For instance, the contention can be identified based on planned resource usage and can be presented to the user as part of the generated output at step 1120′.

With reference now to FIG. 11G, an additional embodiment of the methods of the present disclosure is presented. Method 1100″ of FIG. 11G is similar to method 1100 of FIG. 11A and method 1100′ of FIG. 11B but features an additional step of rectifying any possible contentions.

As before, method 1100″ of FIG. 11G proceeds first at step 1105″ of method 1100″, whereby one or more inputs associated with the parallel biomanufacturing processes to be performed on cell samples within each of a plurality of cartridges may be received. In embodiments, the one or more inputs may include an introduction rate of cartridges to the workcell, types of instruments within the workcell, a number of each type of instrument within the workcell, different states of the instruments within the workcell (e.g., vacant, occupied), one or more process steps associated with each instrument, each cartridge, and/or each process workflow, and allowable variation within each of the one or more process steps associated with each instrument, each cartridge, and/or each process workflow. For instance, the allowable variation can include timing margins for each of the one or more process steps. The timing margins can indicate which of the one or more process steps are exchangeable, distributable, and the like. As will be described later, the simulation can utilize these timing margins when reaching an optimal solution. Additionally, or to this end, the one or more inputs may include requirements and constraints based on a desired outcome of the biomanufacturing processes. For example, a desired outcome (e.g., a target result) may include a desired cell product selected from any of the following: hematopoietic stem cells (HSCs), chimeric antigen receptor (CAR) T cells, NK cells, tumor infiltrating lymphocytes (TILs), T-cell receptors (TCRs), regulatory T cells (T regs), gamma delta (γδ) T-cell. It can be appreciated that each of these cellular products have their own unique manufacturing processes, and accordingly, dictate their own unique constraints and requirements.

At sub process 1110″ of method 1100″, a simulation may be performed based on the one or more inputs received at step 1105″. In some embodiments, the simulation may be an analog simulation or a linear simulation. In some embodiments, and as described above, the simulation may be a finite-state machine model, a Markov chain model, a stochastic process model, a queuing theory model, a transaction-level model, and a discrete-event simulator, which models the parallel processes within the workcell as a sequence of events in time.

Based on the simulation, or in some embodiments, as part of the simulation (denoted by dashed lines to step 1120″), different types of outputs can be generated at step 1120″ of method 1100″, thereby enabling observation of the system behavior and the optimization of resource allocation. In embodiments, the different types of outputs may include periodic Gantt charts, biomanufacturing workcell component performance monitoring, and the potential contentions. Concurrently, based on the simulation or, in some embodiments, as part of the simulation, potential contentions can be identified at step 1115″ of method 1100″. The potential contentions may include at least one contention of a phase contention, an instrument contention, and a workflow step contention. The potential contentions may be identified by visual evaluation of the output generated based on the simulation (or as part of the simulation) and/or identified by the processor of the workcell. For instance, the contention can be identified based on planned resource usage and can be presented to the user as part of the generated output at step 1120″.

Further to the above, at step 1125″ of method 1100″, and based on the identified potential contentions and the generated output based on the simulation, planning of the biomanufacturing processes can be optimized. For instance, a logical series of adjustments, either by the processor or by manual intervention, can be made and the simulation can be performed again. In certain instances, the adjustment could be made by a user to one or more of the inputs to the simulation, such as a constraint on a particular process variation. In another instance, the adjustment could be made by the processor to one or more of the inputs to the simulation dictating entry of a subsequent cartridge and/or adjusting timing margins. To this end, a dashed line between 1125″ and 1105″ of method 1100″ indicates that optimization may require iterative modifications to the one or more inputs, simulation, and output generation. Optimization proceeds until no additional contentions can be identified within the plan.

With reference to the periodic Gantt charts described above, it should be appreciated that the periodicity of the intervals is not constrained to 24-hours. Other timings such as 9-hours, 12-hours, 15-hours, 18-hours, 21-hours, 27-hours, 30-hours, 33-hours, or 36-hours may be beneficial for different processes and/or workflows.

As shown above, periodic Gantt plotting may allow the user, and/or the processor, to immediately see where contention will occur and to optimize the process to mitigate it. With reference now to FIG. 13, an output, based on the simulation, comprising a periodic Gantt chart of workflow processes versus time, is shown. The periodic Gantt chart of FIG. 13 depicts a partial optimization. Data is plotted vertically in 24-hour “periods” with X-axis numbers representing the planning interval (which for 24-hour periods is the same as the processing day). The mixed hue “groups” represent processes within the feed through, the cell separation system, the sterile liquid transfer instrument, and the electroporation system. Specifically, GR is the feed through, YL is the sterile liquid transfer instrument, OR is the cell separation system, PU is the electroporation system, and BL is the bioreactor. The longer BLf periods are bioreactor intervals from 24-48 hours. A number of observations can be made. It can be observed that during the first planning interval (“Day 1”), cartridge introduction is followed by sterile liquid transfer instrument and cell separation system activity. During the second planning interval (“Day 2”), the cartridge is entirely within a bioreactor. Thus, if a cartridge were to be introduced during the second planning interval at the same time as during the first planning interval, the first cartridge will be in a bioreactor and so there will be no contention during cartridge introduction. Next, introducing a cartridge during the third planning interval is unlikely to result in a contention, unless initial processing runs long and is not accounted for (as is described above with relation to extending groups to maximum time). In this case, it is likely the first cartridge will be delayed. Of course, when optimized as described herein, timing margin for the third planning interval can be adjusted to tolerate other cartridge delays without impact (i.e., the note ‘partial optimization’ of this workflow). Lastly, as described above, the group of activities during the third planning interval for the first cartridge can occur at the same time during the fourth planning period for the second cartridge, during the fifth planning period for the third cartridge, and so on, and will not conflict with any of the other activities in the 10-13 hour timeframe on the Y-axis. This is depicted in FIG. 14, which shows three cartridges entered during each planning interval over three consecutive days. In each case, the busy periods (hued) occur when the prior cartridges are in the bioreactor and the other processing instruments are available without contention. Various instruments are utilized throughout the next planning intervals until exit functions are executed during the final planning interval (“Day 7”).

Though depicted in FIG. 13 as only one cartridge and as only three cartridges in FIG. 14, it should be appreciated that, based system constraints dictated by the one or more inputs (e.g., # of bioreactor instruments), there may be any number of cartridges introduced into the workcell as long as contentions can be avoided. In an embodiment, the number of cartridges able to be introduced into the workcell is about equal to the number of bioreactors within the workcell. To this end, more than one bioreactor (which would be indicated with the one or more inputs described above) may be present, and so it is possible for multiple cartridges to be present. For example, if a new cartridge were to be introduced within the plan of FIG. 13, it may appear that there is a contention between 8-9:30 as both cartridges attempt to utilize the bioreactor. However, there is likely more than one bioreactor, thus there is no contention.

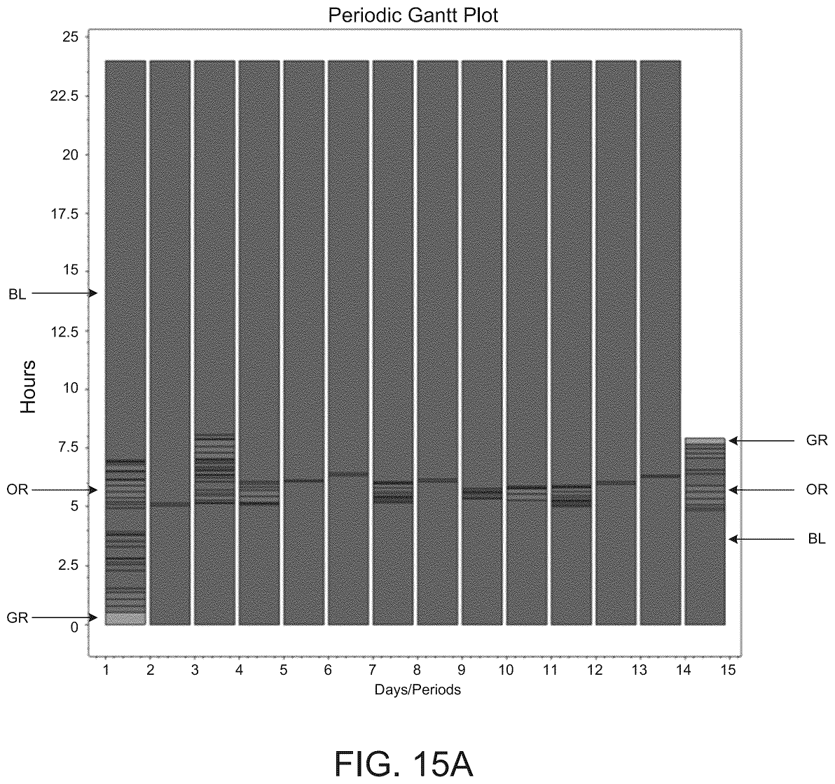

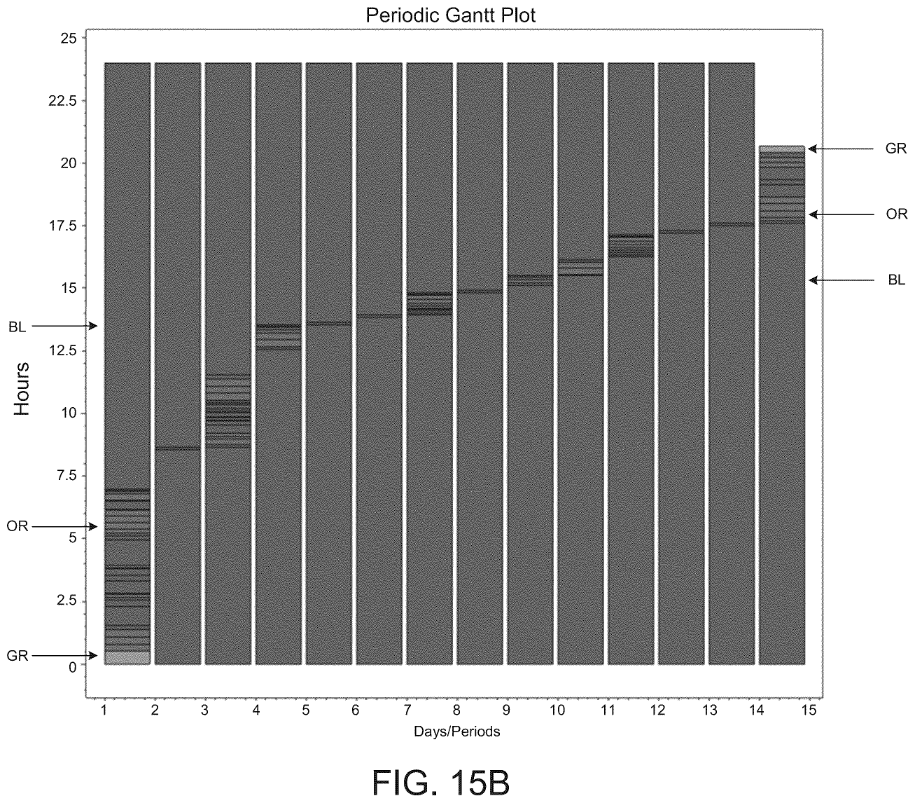

With reference now to the periodic Gantt plots of FIGS. 15A-B, wherein the x-axis represents the days (or periods), the y-axis represents the number of hours in each day (24 hours), and the hues represent the various stations and instruments (e.g., BL is the bioreactor, OR is the sterile liquid transfer instrument, GR is the feed through), a workcell with one bioreactor is shown. In FIG. 15A, it can be observed that, while no contention presently exists, planning instrument utilization in this way on consecutive days is likely to prevent the introduction of additional cartridges. As was described with reference to previous Drawings, if the instrument utilization for a first cartridge on day 1 is repeated for a second cartridge introduced on day 2, a contention is likely to occur, as both cartridges would require the use of the sterile liquid transfer instrument at the same time. In contrast, FIG. 15B depicts a plan for instrument utilization on the workcell that likely ensures additional cartridges can be introduced at days 1, 2, 3, assuming there are sufficient numbers of bioreactors and assuming the workflows are the same.

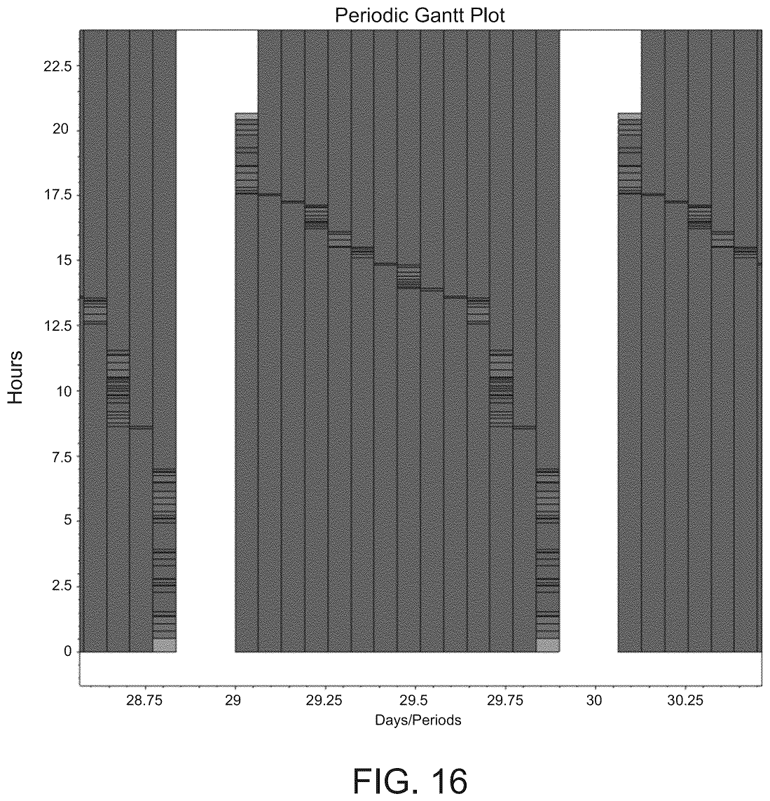

FIG. 16 depicts a subset of a periodic Gantt plot for a workcell having 14 bioreactors accommodating 14 cartridges. As can be seen in FIG. 16, using the methods described herein, the planned instrument utilization results in zero contentions across day 24, even with the increased number of bioreactors in the system.

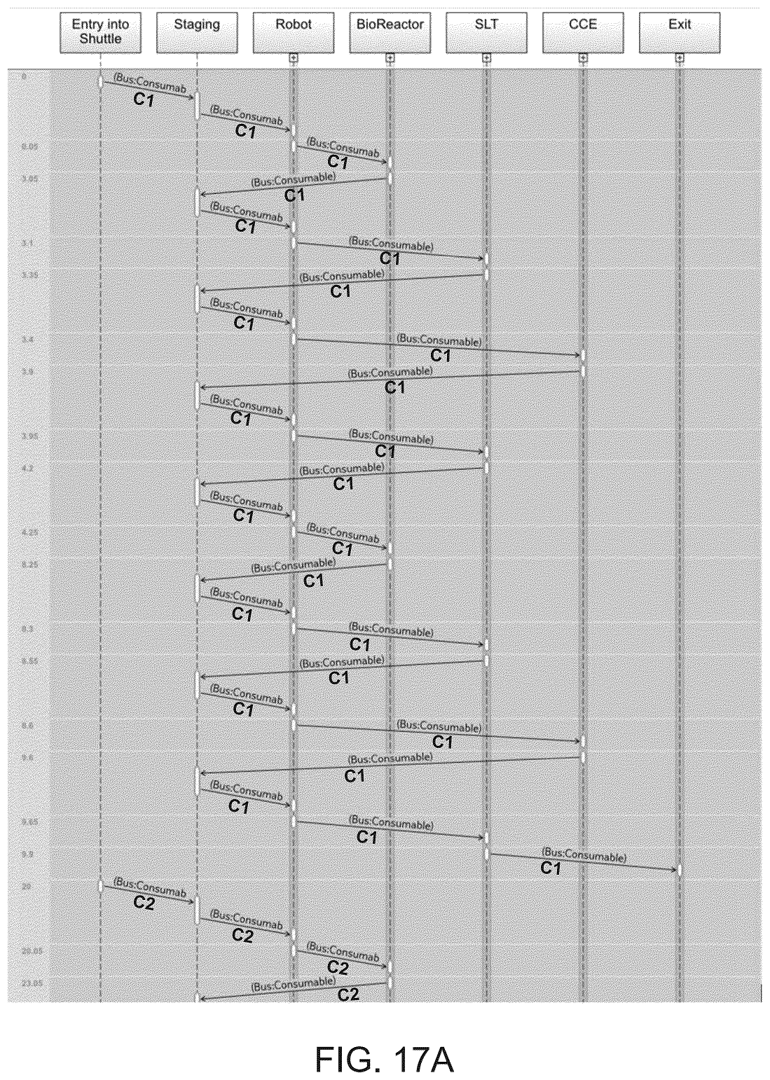

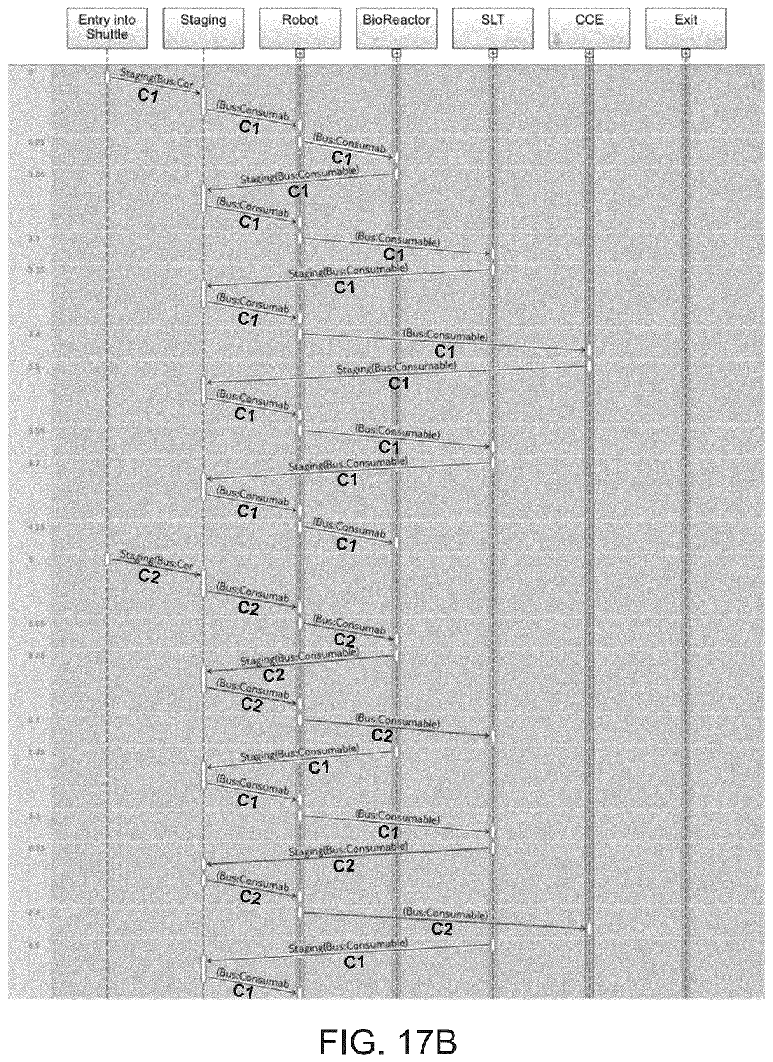

FIGS. 17A and 17B depict another way of visualizing the path of a cartridge within the workcell based on the simulation. The path of each of two cartridges (C1, C2) is traced from position to position, or instrument to instrument, from the entry into the workcell. The sequence runs from top to bottom. Time is not represented linearly on the y-axis but is instead modified in order to better visualize the path of each cartridge. In FIG. 17A, C1 completes a workflow before C2 is introduced to the workcell. In FIG. 17B, C2 is introduced to the workcell before C1 completes its workflow. Each of these visualizations can be used as a tool to investigate and/or debug identified contention issues.

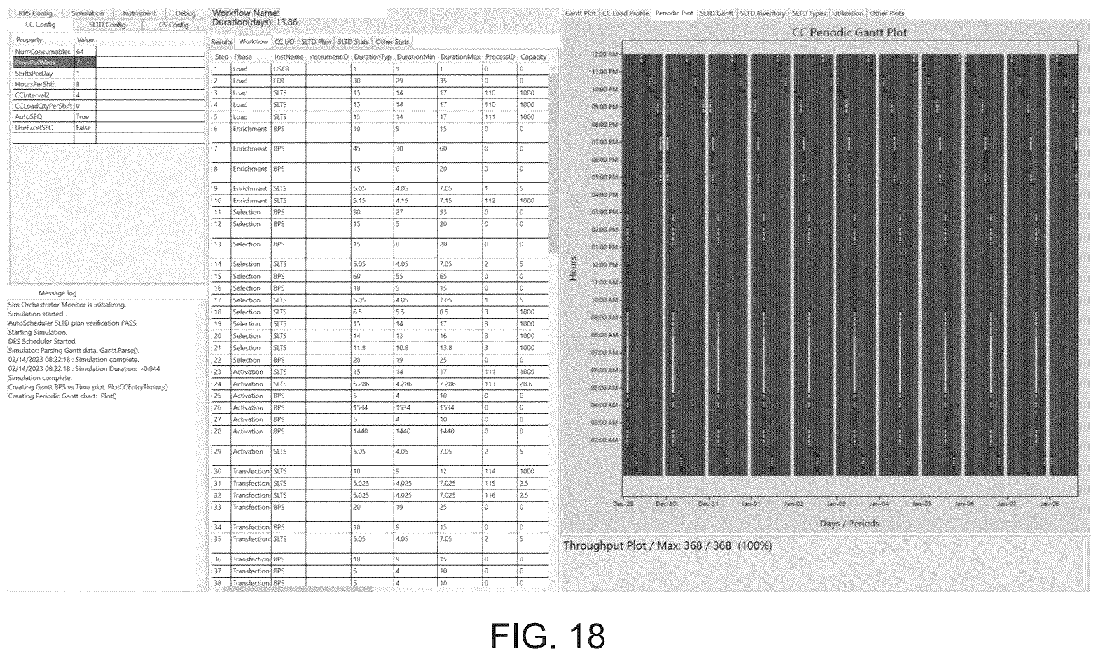

In some embodiments, and as shown in FIG. 18, the methods described with reference to FIGS. 11A-G can additionally include a step of generating a graphical user interface (GUI) to display the generated output to a user. In some embodiments, the step of generating the GUI can be outside of the methods of FIGS. 11A-G but based on the simulation and generated outputs therefrom.

As shown in FIG. 18, the GUI may include the following sections—simulation configuration data, simulation input/output (I/O) tabular data, and graphical outputs. In an embodiment, the simulation configuration data provides system configuration data such as start time, workflow type, and numbers of and types of instruments within the workcell, and the like. In an embodiment, the simulation I/O tabular data includes data similar to that described with reference to FIG. 12. For instance, the simulation I/O tabular data may include processing information such as processing time and expected deviation in hours that could occur (e.g., due to contentions/deadlocks). Further, the simulation I/O tabular data can include an “ID” column that identifies a step of a particular process (e.g., cartridge is being processed by a bioreactor; cartridge receives liquid transfer via a sterile liquid transfer device), a “time in” that identifies the projected start time of the process, a “time out” that identifies the projected finish time of the process, a “time to process” that identifies the projected time from start to finish, and a “Dev (hrs)” that identifies the projected process deviation. In an embodiment, the graphical outputs may include various views of data across several tabs. For instance, the graphical outputs may include Gantt charts (e.g., periodic Gantt charts), sterile liquid transfer instrument performance monitoring, and system contentions presented in various forms based on phase, instrument, and workflow step.

All embodiments of any aspect of the disclosure can be used in combination, unless the context clearly dictates otherwise.

While embodiments of the present invention have been shown and described herein, those skilled in the art will understand that such embodiments are provided by way of example only. Numerous embodiments, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### NUMBERED EMBODIMENTS OF THE INVENTION

Notwithstanding the appended claims, the disclosure sets forth the following numbered embodiments:
(1) A system for planning parallel biomanufacturing processes, the system comprising a plurality of cartridges, each containing a cell sample, a biomanufacturing workcell configured to receive the plurality of cartridges and comprising a plurality of instruments configured to interface with respective modules of each one of the plurality of cartridges to perform biomanufacturing processes on the cell sample therein, and a computing device comprising a processor configured to receive one or more inputs associated with the biomanufacturing processes to be performed on the cell sample within each of the plurality of cartridges, simulate the biomanufacturing processes based on the received one or more inputs, identify potential contentions based on the simulation, and generate an output based on the one or more inputs associated with the biomanufacturing processes, the simulation, and the identified potential contentions, wherein the output comprises a chart including a displayed series of intervals, wherein each interval represents a displayed series of time periods, and wherein the plurality of instruments are displayed as a function of the displayed series of interval and the displayed series of time periods.
(2) The system of (1), wherein the plurality of instruments include at least one bioreactor instrument and a sterile liquid transfer instrument.
(3) The system of either (1) or (2), wherein the biomanufacturing workcell further comprises one or more robotic arms configured to move each of the plurality of cartridges within the biomanufacturing workcell to or from locations associated with each of the plurality of instruments.
(4) The system of any one of (1) to (3), wherein the one or more inputs comprise at least one input selected from the group consisting of: a type of each of the plurality of instruments, a number of each of the plurality of instruments, and constraints related to process steps associated with a particular biomanufacturing process.
(5) The system of any one of (1) to (4), wherein the simulation is a discrete-event simulation.
(6) The system of any one of (1) to (5), wherein the potential contentions comprise at least one contention selected from the group consisting of: a phase contention, an instrument contention, and a workflow step contention.
(7) The system of any one of (1) to (6), wherein the output comprises a periodic Gantt chart.
(8) The system of any one of (1) to (7), wherein the system further comprises a graphical user interface in communication with the processor of the computing device.
(9) The system of any one of (1) to (8), wherein the processor is further configured to display the generated output via the graphical user interface.
(10) The system of any one of (1) to (9), wherein the processor is further configured to display system configuration parameters via the graphical user interface, the system configuration parameters including a number of at least one bioreactor of the plurality of instruments.
(11) The system of any one of (1) to (10), wherein the processor is further configured to display at least a portion of the one or more inputs associated with the parallel biomanufacturing processes via the graphical user interface.
(12) The system of any one of (1) to (11), wherein the processor is further configured to display at least a portion of the generated output via the graphical user interface, the at least a portion including graphical outputs.
(13) The system of any one of (1) to (12), wherein the graphical outputs include at least one output selected from the group consisting of: periodic Gantt charts, biomanufacturing workcell component performance monitoring, and the potential contentions.
(14) The system of any one of (1) to (13), wherein the potential contentions comprise at least one contention selected from the group consisting of: a phase contention, an instrument contention, and a workflow step contention, and when the graphical outputs include the potential contentions, a form of a particular graphical output is based on a type of the at least one contention.
(15) The system of any one of (1) to (14), wherein the plurality of instruments further includes a feedthrough.
(16) The system of any one of (1) to (15), wherein the plurality of instruments further includes at least one bioreactor instrument, at least one sterile liquid transfer instrument, an electroporation instrument, a counterflow centrifugal elutriation instrument, and a magnetic-activated cell selection instrument.
(17) The system of any one of (1) to (16), wherein a number of the plurality of cartridges is equal to a number of the at least one bioreactor of the plurality of instruments.
(18) The system of any one of (1) to (17), wherein the parallel biomanufacturing processes are automated.
(19) The system of any one of (1) to (18), wherein the biomanufacturing workcell further comprises a fluid storage vault.
(20) The system of any one of (1) to (19), wherein the biomanufacturing workcell further comprises a fluid storage vault comprising one or more closed volume fluid devices and the plurality of instruments comprises a sterile liquid transfer instrument, the one or more robotic arms being further configured to move the one or more closed volume fluid devices between the fluid storage vault and the sterile liquid transfer instrument.
(21) The system of any one of (1) to (20), wherein the biomanufacturing workcell further comprises a fluid storage vault comprising one or more closed volume fluid devices, the one or more robotic arms being further configured to move the plurality of cartridges to and couple each of the plurality of cartridges to the fluid storage vault, and wherein waste generated by the plurality of cartridges is transferred to a corresponding closed volume fluid device within the fluid storage vault.
(22) The system of any one of (1) to (21), wherein the biomanufacturing workcell further comprises a fluid storage vault comprising one or more closed volume fluid devices, the one or more robotic arms being further configured to move the plurality of cartridges to and couple each of the plurality of cartridges to the fluid storage vault, and wherein cell therapy samples generated by the plurality of cartridges are transferred to a corresponding closed volume fluid device within the fluid storage vault.
(23) The system of any one of (1) to (22), wherein the fluid storage vault comprises one or more closed volume fluid devices, at least one of the one or more closed volume fluid devices being a fluid vessel.
(24) The system of any one of (1) to (23), wherein at least a portion of the plurality of instruments are shared instruments utilized by at least a portion of the plurality of cartridges.
(25) The system of any one of (1) to (24), wherein the process steps associated with the particular biomanufacturing process include at least one process selected from the group consisting of: cultivation in a bioreactor instrument, electroporation within an electroporation instrument, cell selection within a magnetic-activated cell selection instrument.
(26) The system of any one of (1) to (25), wherein the constraints related to the process steps associated with the particular biomanufacturing process include at least one constraint selected from the group consisting of average duration of the particular process step, minimum duration of the particular process step, and maximum duration of the particular process step.
(27) The system of any one of (1) to (26), wherein the periodic Gantt chart has a periodicity of 24 hours.
(28) The system of any one of (1) to (27), wherein a last process step of an initial period ends at a time position within the initial period that is earlier than a time position within a subsequent period during which a first process step of the subsequent period begins.
(29) The system of c any one of (1) to (28), wherein a single cartridge is introduced to the biomanufacturing workcell during a given period.
(30) The system of any one of (1) to (29), wherein each of the plurality of cartridges is introduced to the biomanufacturing workcell via the feedthrough.
(31) A method for identifying workflow conflicts in parallel biomanufacturing processes, the system comprising receiving, by a processor of a computing device, one or more inputs associated with biomanufacturing processes to be performed on cell samples within each of a plurality of cartridges, simulating the parallel biomanufacturing processes based on the received one or more inputs, identifying potential contentions based on the simulation, and generating an output based on the simulated parallel biomanufacturing process and the identified potential contentions, wherein the output comprises a chart wherein a plurality of instruments are displayed as a function of a series of intervals and a series of time periods, wherein the parallel biomanufacturing processes are performed on the cell samples within the plurality of cartridges in coordination with a biomanufacturing workcell comprising a plurality of instruments configured to interface with respective modules of each one of the plurality of cartridges to perform the biomanufacturing processes.
(32) The method of (31), wherein the biomanufacturing workcell further comprises one or more robotic arms configured to move each of the plurality of cartridges within the biomanufacturing workcell to or from locations associated with each of the plurality of instruments.
(33) The method of (32), further comprising optimizing the simulation based on the identified potential contentions.
(34) The method of (33), wherein the optimizing includes adjusting an introduction rate of the plurality of cartridges to the biomanufacturing workcell via a feedthrough of the biomanufacturing workcell.

What is claimed herein is:

1. A system for planning parallel biomanufacturing processes, the system comprising:
a plurality of cartridges, each containing a cell sample;
a biomanufacturing workcell configured to receive the plurality of cartridges and comprising a plurality of instruments configured to interface with respective modules of each one of the plurality of cartridges to perform biomanufacturing processes on the cell sample therein; and
a computing device comprising a processor configured to
receive one or more inputs associated with the biomanufacturing processes to be performed on the cell sample within each of the plurality of cartridges,
simulate the biomanufacturing processes based on the received one or more inputs,
identify potential contentions based on the simulation, and
generate an output based on the one or more inputs associated with the biomanufacturing processes, the simulation, and the identified potential contentions, wherein the output comprises a chart including a displayed series of intervals, wherein each interval represents a displayed series of time periods, and wherein the plurality of instruments are displayed as a function of the displayed series of interval and the displayed series of time periods.

2. The system of claim 1, wherein the plurality of instruments include at least one bioreactor instrument and a sterile liquid transfer instrument.

3. The system of claim 1, wherein the biomanufacturing workcell further comprises
one or more robotic arms configured to move each of the plurality of cartridges within the biomanufacturing workcell to or from locations associated with each of the plurality of instruments.

4. The system of claim 1, wherein the one or more inputs comprise at least one input selected from the group consisting of: a type of each of the plurality of instruments, a number of each of the plurality of instruments, and constraints related to process steps associated with a particular biomanufacturing process.

5. The system of claim 1, wherein the simulation is a discrete-event simulation.

6. The system of claim 1, wherein the potential contentions comprise at least one contention selected from the group consisting of: a phase contention, an instrument contention, and a workflow step contention.

7. The system of claim 1, wherein the output comprises a periodic Gantt chart.

8. The system of claim 1, wherein the system further comprises a graphical user interface in communication with the processor of the computing device.

9. The system of claim 8, wherein the processor is further configured to display the generated output via the graphical user interface.

10. The system of claim 8, wherein the processor is further configured to display system configuration parameters via the graphical user interface, the system configuration parameters including a number of at least one bioreactor of the plurality of instruments.

11. The system of claim 8, wherein the processor is further configured to display at least a portion of the one or more inputs associated with the parallel biomanufacturing processes via the graphical user interface.

12. The system of claim 8, wherein the processor is further configured to display at least a portion of the generated output via the graphical user interface, the at least a portion including graphical outputs.

13. The system of claim 12, wherein the graphical outputs include at least one output selected from the group consisting of: periodic Gantt charts, biomanufacturing workcell component performance monitoring, and the potential contentions.

14. The system of claim 13, wherein the potential contentions comprise at least one contention selected from the group consisting of: a phase contention, an instrument contention, and a workflow step contention, and when the graphical outputs include the potential contentions, a form of a particular graphical output is based on a type of the at least one contention.

15. The system of claim 1, wherein the plurality of instruments further includes a feedthrough.

16. The system of claim 1, wherein the plurality of instruments further includes at least one bioreactor instrument, at least one sterile liquid transfer instrument, an electroporation instrument, a counterflow centrifugal elutriation instrument, and a magnetic-activated cell selection instrument.

17. The system of claim 1, wherein a number of the plurality of cartridges is equal to a number of the at least one bioreactor of the plurality of instruments.

18. The system of claim 1, wherein the parallel biomanufacturing processes are automated.

19. The system of claim 1, wherein the biomanufacturing workcell further comprises a fluid storage vault.

20. The system of claim 3, wherein the biomanufacturing workcell further comprises a fluid storage vault comprising one or more closed volume fluid devices and the plurality of instruments comprises a sterile liquid transfer instrument, the one or more robotic arms being further configured to move the one or more closed volume fluid devices between the fluid storage vault and the sterile liquid transfer instrument.

21. The system of claim 3, wherein the biomanufacturing workcell further comprises a fluid storage vault comprising one or more closed volume fluid devices, the one or more robotic arms being further configured to move the plurality of cartridges to and couple each of the plurality of cartridges to the fluid storage vault, and wherein waste generated by the plurality of cartridges is transferred to a corresponding closed volume fluid device within the fluid storage vault.

22. The system of claim 3, wherein the biomanufacturing workcell further comprises a fluid storage vault comprising one or more closed volume fluid devices, the one or more robotic arms being further configured to move the plurality of cartridges to and couple each of the plurality of cartridges to the fluid storage vault, and wherein cell therapy samples generated by the plurality of cartridges are transferred to a corresponding closed volume fluid device within the fluid storage vault.

23. The system of claim 19, wherein the fluid storage vault comprises one or more closed volume fluid devices, at least one of the one or more closed volume fluid devices being a fluid vessel.

24. The system of claim 1, wherein at least a portion of the plurality of instruments are shared instruments utilized by at least a portion of the plurality of cartridges.

25. The system of claim 6, wherein the process steps associated with the particular biomanufacturing process include at least one process selected from the group consisting of: cultivation in a bioreactor instrument, electroporation within an electroporation instrument, cell selection within a magnetic-activated cell selection instrument.

26. The system of claim 6, wherein the constraints related to the process steps associated with the particular biomanufacturing process include at least one constraint selected from the group consisting of:
average duration of a particular process step,
minimum duration of the particular process step, and
maximum duration of the particular process step.

27. The system of claim 7, wherein the periodic Gantt chart has a periodicity of 24 hours.

28. The system of claim 7, wherein a last process step of an initial period ends at a time position within the initial period that is earlier than a time position within a subsequent period during which a first process step of the subsequent period begins.

29. The system of claim 7, wherein a single cartridge is introduced to the biomanufacturing workcell during a given period.

30. The system of claim 15, wherein each of the plurality of cartridges is introduced to the biomanufacturing workcell via the feedthrough.

31. A method for identifying workflow conflicts in parallel biomanufacturing processes, the system comprising:
receiving, by a processor of a computing device, one or more inputs associated with biomanufacturing processes to be performed on cell samples within each of a plurality of cartridges;
simulating the parallel biomanufacturing processes based on the received one or more inputs;
identifying potential contentions based on the simulation; and
generating an output based on the simulated parallel biomanufacturing process and the identified potential contentions, wherein the output comprises a chart wherein a plurality of instruments are displayed as a function of a series of intervals and a series of time periods,
wherein the parallel biomanufacturing processes are performed on the cell samples within the plurality of cartridges in coordination with a biomanufacturing workcell comprising a plurality of instruments configured to interface with respective modules of each one of the plurality of cartridges to perform the biomanufacturing processes.

32. The method of claim 31, wherein the biomanufacturing workcell further comprises
one or more robotic arms configured to move each of the plurality of cartridges within the biomanufacturing workcell to or from locations associated with each of the plurality of instruments.

33. The method of claim 31, further comprising
optimizing the simulation based on the identified potential contentions.

34. The method of claim 33, wherein the optimizing includes adjusting an introduction rate of the plurality of cartridges to the biomanufacturing workcell via a feedthrough of the biomanufacturing workcell.
